SEQUENCE LISTING (5 sequences)

SEQ ID NO: 1 - DNA, 25 bp, Artificial Sequence
  Synthetic
ctcctttgtatcttccataagtagc

SEQ ID NO: 2 - DNA, 25 bp, Artificial Sequence
  Synthetic
ttcgtatcttcgctagcttatacac

SEQ ID NO: 3 - protein, 4 aa, Homo sapiens
KDMV

SEQ ID NO: 4 - protein, 4 aa, Homo sapiens
DTSL

SEQ ID NO: 5 - protein, 4 aa, Homo sapiens
DIRL

(19) United States
(12) Patent Application Publication
(10) Pub. No.: US 2005/0158280 A1
(43) Pub. Date: Jul. 21, 2005

(54) Method of reducing angiogenesis

(76) Inventors: Simon C. Robson, Weston, MA (US); Christian Goepfert, Hamburg (DE); Christian Sundberg, Uppsala (SE); Tomokazu Hoshi, Asahikawa (JP)

Correspondence Address: CLARK & ELBING LLP, 101 FEDERAL STREET, BOSTON, MA, 02110, US

(21) Appl. No.: 10/870,388

(22) Filed: Jun. 17, 2004

Related U.S. Application Data
(63) Continuation-in-part of application No. PCT/US02/40471, filed on Dec. 17, 2002.
(60) Provisional application No. 60/341,370, filed on Dec. 17, 2001.

(51) Int. Cl. A61K 48/00; C12Q 1/68

(52) U.S. Cl. 424/93.2; 514/44; 435/6

(57) ABSTRACT

The invention features methods of identifying a compound capable of modulating angiogenesis. Further features of the invention are methods of promoting or inhibiting angiogenesis. Methods for the diagnosis of a CD39-associated condition and for determining the prognosis of a patient diagnosed with a CD39-associated condition are also disclosed.

### CROSS REFERENCE TO RELATED APPLICATIONS

This application is a continuation-in-part of International Application No. PCT/US02/4047 1, filed Dec. 17, 2002, which was published in English under PCT Article 21(2), and which claims the benefit of the filing date of U.S. Provisional Application No. 60/341,370 filed Dec. 17, 2001; each of these disclosures is incorporated by reference in their entirety.

### STATEMENT OF FEDERALLY SPONSORED RESEARCH

This research has been sponsored in part by NIH grant numbers HL-57307 and HL-63972. The government has certain rights to the invention.

### BACKGROUND OF THE INVENTION

The normal vascular endothelium maintains blood fluidity and flow by inhibiting coagulation and platelet activation, and by promoting fibrinolysis. Quiescent endothelial cells are considered to directly express natural anticoagulants and thromboregulatory factors. In general, thrombosis usually develops secondary to the overwhelming of these antithrombotic mechanisms. This may occur following the heightened generation of locally produced mediators, including extracellular nucleotides, cytokines, and activated complement components.

Extracellular nucleotides present in the blood due to, e.g., arterial vascular injury can influence cardiac function, vasomotor responses, platelet activation, thrombosis, and inflammatory processes. When present, extracellular nucleotides bind to and stimulate purinergic/pyrimidinergic type-2 (P2) receptors. This stimulation initiates G protein-coupled signaling pathways and results in activation of platelets, endothelial cells (EC), monocyte/macrophages, and leukocytes and can culminate in vascular thrombosis and inflammation in vivo. ATP and ADP appear to regulate hemostasis through the activation of platelet P2 receptors. ADP is a major platelet recruiting and activating factor, whereas ATP acts as a competitive agonist of ADP for platelet P2 receptors. This latter protective action of ATP may limit the formation of intravascular platelet aggregation and help localize thrombus formation to areas of vascular damage.

Another key regulator of hemostasis are the ectonucleotidases of the nucleoside triphosphate diphosphohydrolase (NTPDase)/CD39 family. NTPDases are Ca²⁺/Mg²⁺ dependent ectoenzymes that hydrolyze nucleoside 5′-triphosphates and nucleoside 5′-diphosphates. This ecto-enzyme can be shown to efficiently bind and hydrolyze extracellular ATP (and ADP) to AMP. One important function of ectonucleotidases is the modulation of P2-receptor-mediated signaling by the removal of extracellular ATP and ADP and related nucleotides. The ultimate generation of extracellular adenosine will not only abrogate or terminate nucleotide-mediated effects, but will also activate adenosine receptors, with often opposing (patho)physiological effects. The regulated dephosphorylation of extracellular nucleotides by ectonucleotidases may be critical for appropriate purinergic/pyrimidinergic signaling and metabolic homeostasis. One example is the regulation of the platelet activation response by maintaining a balance in the relative concentration of ATP and ADP in the blood.

Pathways of nucleotide-mediated signaling are further complicated, however, by P2-receptor desensitization phenomena. P2 receptor desensitization occurs in the presence of excess nucleotide di- and tri-phosphates and may involve phosphorylation of the receptor by multiple protein kinases, which can result in downregulation and a decrease in cell surface localization of P2 receptors. Thus, nucleotides may also directly limit NTP- and NDP-mediated activation of P2 receptors resulting in, for example, decreased activation of platelets.

Previous studies have proposed the use of CD39 as a therapeutic agent for preventing platelet aggregation. Expression of a CD39 antisense oligonucleotide has been shown to decrease the ATPase activity of CD39 in vitro and may help to explain how the loss of vascular ATPDase activity modulates progression of vascular injury (Imai et al., Biochemistry 38:13473-13479 (1999)). Furthermore, the use of adenovirus-mediated expression of CD39 has been shown to augment the ATPDase activity of vessel walls (Gangadharan et al., Surgery 130:296-303 (2001)).

Angiogenesis is a highly complex multistep phenomenon that incorporates both formation of new capillaries and expansion or extension of existing blood vessels and may also be regulated by P2 receptor-mediated activity. New vessel growth may be modulated by activation of P2 receptors on monocyte/macrophages and endothelial cells, which secrete angiogenic factors and metalloproteases that facilitate endothelial cell migration. Evidence of nucleotide and P2 receptor regulation of angiogenesis comes from the binding of angiostatin, a proteolytic fragment of plasminogen and a potent antagonist of angiogenesis, to ATP synthase, which is expressed on endothelial cells and has been shown to mediate antiangiogenic effects.

Because angiogenesis, and vascularization in general, is critical to the progression of various diseases, for example, cancer, there exists a need for therapeutics that effectively control vascularization.

### SUMMARY OF THE INVENTION

We have discovered that CD39, also known as nucleoside triphosphate diphosphohydrolase-1, is the major NTPDase in vascular endothelial cells. Nucleotide triphosphates activate P2 receptors on monocyte, macrophage, and endothelial cells and promote the adhesion and migration of these cells to sites undergoing angiogenesis. The presence of nucleotide diphosphates, however, activates platelets and leads to aggregation and thrombus formation, thus preventing neovascularization. We have found that NTPDase/CD39 acts to regulate the concentration of nucleotide di- and triphosphates, thereby maintaining the balance between angiogenesis and intravascular coagulation.

We have identified the role of CD39 in angiogenesis using a mouse model wherein cd39 has been deleted. Using this model, we have determined that, in the absence of CD39, P2 receptors are desensitized and downregulated, and are therefore unable to mediate adhesion and migration of monocyte/macrophages (or endothelial cells) to the site of blood vessel formation. In the absence of these cells, neovascularization fails to occur. Using cd39-null mice, we demonstrate that monocyte, macrophage, and endothelial cells are unable to migrate into MATRIGEL® plugs containing growth factors or into an area containing implanted tumor cells to initiate angiogenesis. When the same experiment is performed using wildtype mice, angiogenesis is observed. This finding demonstrates that CD39 is required for initiating angiogenesis. This requirement for CD39 can be extrapolated to diseases involving of angiogenesis, including cancer, rheumatoid arthritis, diabetic retinopathy, and inflammatory bowel disease.

Based on our discoveries, we provide methods of screening compounds for their effectiveness in modulating the angiogenic activity of CD39 using various developed assays of CD39 activity. These assays include, but are not limited to, protein expression assays, NTPDase activity assays, MATRIGEL® transmigration assays, platelet activation assays, and in vivo angiogenesis assays.

Furthermore, we propose methods of modulating the activity of CD39 to reduce angiogenesis in patients suffering from a condition exacerbated by an increase in angiogenesis. Based on our findings we can also propose a method of enhancing angiogenesis using the activity of CD39 in patients in need thereof, by promoting angiogenesis for the treatment, stabilization, or prevention of, for example, cardiovascular disease, peripheral vascular disease, tissue organ engraftment, or sequelae of ischemic reperfusion injury.

Accordingly, the invention features a method of identifying a compound capable of modulating angiogenesis in a subject that involves (a) exposing a cell expressing CD39 to a compound and (b) assaying CD39 biological activity, wherein a decrease in CD39 biological activity in the cell, relative to CD39 biological activity in a cell not exposed to the compound, indicates that the compound is potentially capable of decreasing angiogenesis, and an increase in CD39 biological activity in the cell, relative to CD39 biological activity in a cell not exposed to the compound, indicates that the compound is potentially capable of increasing angiogenesis. In a desired embodiment, CD39 biological activity is the phosphohydrolysis of nucleoside diphosphate (NDP) or triphosphate (NTP). In another desired embodiment, the nucleoside diphosphate is selected from adenosine diphosphate (ADP) or uridine diphosphate (UDP), or said nucleoside triphosphate is selected from adenosine triphosphate (ATP) or uridine triphosphate (UTP). In yet another embodiment, the cell is a monocyte, macrophage, endothelial cell, or cancer cell.

Another aspect of the invention features a method of identifying a compound that is capable of modulating CD39-associated angiogenesis in a subject that involves injecting MATRIGEL® containing one or more growth factors and a compound into a CD39 null mouse and assaying for the ingrowth of blood vessels into the MATRIGEL®, wherein an increase or a decrease in the ingrowth of blood vessels into the MATRIGEL®, relative to the ingrowth of blood vessels into MATRIGEL® lacking the compound, indicates that the compound is capable of promoting or inhibiting, respectively, angiogenesis. In an embodiment, the one or more growth factors are selected from vascular endothelial growth factor (VEGF), spingosine-1-phosphate (SPP), and fibroblast growth factor (FGF). In various embodiments, the assaying for the ingrowth of blood vessels is determined after incubating the MATRIGEL® in the mouse for between 1 and 60 days; more desirably between 7 and 21 days. In yet another embodiment, the MATRIGEL® is injected into the mouse at a concentration of between 1 mg/mL and 20 mg/mL. More desirably, the MATRIGEL® is injected into the mouse at a concentration of between 5 mg/mL and 15 mg/mL. In another embodiment, the growth factors are used at a concentration of between 0.05 μg/mL and 1000 μg/mL.

The invention also features a method of decreasing angiogenesis in a subject in need thereof, consisting of administering a compound that decreases CD39 biological activity in an amount sufficient to decrease angiogenesis. In an embodiment, CD39 biological activity is the phosphohydrolysis of nucleoside diphosphate or triphosphate. In another embodiment, the compound is selected from the group consisting of a nucleoside analog, a peptide, an antibody, and a CD39 antisense RNA. In a desired embodiment, the compound is antisense CD39 RNA capable of decreasing CD39 biological activity in a cell expressing CD39. In yet another embodiment, the subject is a human subject. In still another embodiment, the subject has one or more of the following: cancer, rheumatoid arthritis, diabetic retinopathy, inflammatory bowel disease, or chronic radiation-induced proctitis.

The invention also features a method of promoting angiogenesis in a subject in need thereof, consisting of administering a compound that increases CD39 biological activity in an amount sufficient to promote angiogenesis. In an embodiment, CD39 biological activity is the phosphohydrolysis of nucleoside diphosphate or triphosphate. In another embodiment, the compound is selected from a CD39 transgene in an expressible genetic construct or a peptide mimetic of CD39. In a desired embodiment, the CD39 transgene is administered by a viral vector, for example, an adenoviral vector. In yet another embodiment, the subject is a human subject. In yet another embodiment, the subject has one or more of the following peripheral vascular disease, cardiovascular disease, tissue organ engraftment rejection, or sequelae of ischemic reperfusion injury. In still another embodiment, the peripheral vascular disease is atherosclerosis, thromboembolic disease, or Buerger's disease (thromboangiitis obliterans). In a further embodiment, the cardiovascular disease is myocardial infarction, heart disease, or coronary artery disease.

The invention features a pharmaceutical composition consisting of CD39 antisense RNA in a pharmaceutically acceptable carrier, wherein the antisense RNA is capable of reducing angtiogenesis in a subject.

The invention also features a method of diagnosing an increased risk of an angiogenesis-associated condition consisting of detecting the level of CD39 biological activity in a subject, wherein an increased or decreased level of CD39 biological activity indicates said subject has an increased risk of an angiogenesis-associated condition. In an embodiment of the method, the angiogenesis-associated condition is cancer or metastasis of cancer, inflammation, inflammatory bowel disease, or chronic radiation-induced proctitis, and detection of an increase in the level of CD39 biological activity indicates that the subject has an increased risk of cancer or metastasis of cancer, inflammation, inflammatory bowel disease, or chronic radiation-induced proctitis. In other embodiments, the angiogenesis-associated condition is peripheral vascular disease (e.g., atherosclerosis, thromboembolic disease, or Buerger's disease (thromboangiitis obliterans)), cardiovascular disease (e.g., myocardial infarction, heart disease, or coronary artery disease), tissue organ engraftment rejection, or sequelae of ischemic reperfusion injury, and detection of a decrease in the level of CD39 biological activity indicates that the subject has an increased risk of peripheral vascular disease (e.g., atherosclerosis, thromboembolic disease, or Buerger's disease (thromboangiitis obliterans)), cardiovascular disease (e.g., myocardial infarction, heart disease, or coronary artery disease), tissue organ engraftment rejection, or sequelae of ischemic reperfusion injury. In yet another embodiment, the level of CD39 biological activity is detected by assaying the level of CD39 mRNA, CD39 protein or the phosphohydrolytic activity of CD39. In a further embodiment, the level of CD39 mRNA, CD39 protein or the phosphohydrolytic activity of CD39 is detected using a biopsy.

Another feature of the invention is a method for determining the prognosis for treatment of an angiogenesis-associated condition in a subject that involves determining the level of CD39 biological activity in a sample from a subject, wherein an increase or decrease in the CD39 biological activity in the sample, relative to the amount of CD39 biological activity in a control sample, determines the prognosis for treatment of an angiogenesis-associated condition in a subject. In an embodiment of the method, the angiogenesis-associated condition is cancer or metastasis of cancer, rheumatoid arthritis, diabetic retinopathy, inflammatory bowel disease, or chronic radiation-induced proctitis and an increase in CD39 biological activity indicates a negative prognosis, while a decrease in CD39 biological activity indicates a positive prognosis. In another embodiment of the method, the angiogenesis-associated condition is peripheral vascular disease, cardiovascular disease, tissue organ engraftment rejection, or sequelae of ischemic reperfusion injury and an increase in CD39 biological activity indicates a positive prognosis, while a decrease in CD39 biological activity indicates a negative prognosis. In yet another embodiment, peripheral vascular disease is atherosclerosis, thromboembolic disease, or Buerger's disease (thromboangiitis obliterans). In still another embodiment, cardiovascular disease is myocardial infarction, heart disease, or coronary artery disease. In a further embodiment, the subject is a human subject. In yet another embodiment, the sample is a biopsy.

### DEFINITIONS

By “angiogenesis” we mean the formation and differentiation of blood vessels.

By “angiogenesis-associated condition,” we mean a condition exacerbated by an increase or decrease in the generation of blood vessels. Examples of angiogenesis-associated pathological conditions that are exacerbated by an increase in the generation of blood vessels are cancer or metastasis of cancer, chronic inflammatory illnesses (e.g., rheumatoid arthritis), inflammatory bowel disease (e.g., Crohn's disease or ulcerative colitis)), diabetic retinopathy, and chronic radiation-induced proctitis. Examples of angiogenesis-associated pathological conditions that are exacerbated by a decrease in the generation of blood vessels (i.e., a loss of blood vessels or the failure to generate new blood vessels) are peripheral vascular disease (e.g., atherosclerosis, thromboembolic disease, Buerger's disease (thromboangiitis obliterans)), cardiovascular disease (e.g., atherosclerosis, heart disease, myocardial infarction, or coronary artery disease), tissue organ engraftment rejection, and sequelae of ischemic reperfusion injury.

By “antisense,” as used herein in reference to nucleic acids, is meant a nucleic acid molecule, regardless of length, that is complementary to the coding strand or mRNA of a target gene, for example, a gene that promotes phosphohydrolysis of nucleoside di- and triphosphates. The antisense nucleic acid is capable of reducing or preventing CD39 expression when present in a cell, for example, a monocyte or macrophage. Such reduction in CD39 expression would decrease the concentration of nucleoside di- and triphosphates that would otherwise interact with P2 receptors. An antisense nucleic acid molecule may decrease the activity of a polypeptide encoded by the target gene. Desirably the decrease is at least 10%, relative to a control, more desirably at least 25%, 50%, or 75%, and most desirably at least 90% or 99% or more. An antisense nucleic acid molecule may comprise from about 8 to 30 nucleotides. An antisense nucleic acid may also contain at least 40, 60, 85, 120, or more consecutive nucleotides that are complementary to a target mRNA or DNA, and may be as long as the full-length target gene or mRNA. The antisense nucleic acid may contain a modified backbone, for example, phosphorothioate, phosphorodithioate, or other modified backbones known in the art, or may contain non-natural internucleoside linkages.

By “biological activity” we mean a polypeptide or other compound having structural, regulatory, or biochemical functions of a naturally occurring molecule.

By “CD39 biological activity” we mean an activity that promotes or inhibits angiogenesis, the regulation of cellular infiltration, or new vessel growth by regulating the level of nucleoside di- and triphosphates via phosphohydrolysis.

By “CD39-associated condition,” we mean a disease or condition exacerbated by CD39 biological activity. A CD39-associated condition may involve the disregulation of CD39 biological activity, such that CD39 biological activity is increased or decreased or such that CD39 expression is increased or decreased. A CD39-associated condition can result from a mutation in a CD39 gene that alters the expression or biological activity of a CD39 nucleic acid molecule or polypeptide. A CD39-associated condition can arise in any vascular tissue in which CD39 is expressed. Examples of a CD39-associated condition are the following: cancer or metastatsis of cancer, a disease associated with chronic inflammatory illnesses (e.g., rheumatoid arthritis), inflammatory bowel disease, diabetic retinopathy, chronic radiation-induced proctitis, peripheral vascular disease (e.g., atherosclerosis, thromboembolic disease, or Buerger's disease (thromboangiitis obliterans)), cardiovascular disease (e.g., myocardial infarction, heart disease, or coronary artery disease), tissue organ engraftment rejection, and sequelae of ischemic reperfusion injury.

By “CD39-mediated increase in angiogenesis” we mean an increase in the formation of blood vessels due to an increase in CD39 expression, e.g., an increase in transcription or translation, or an increase in CD39 biological activity, e.g., an increase in phosphohydrolysis of extracellular nucleoside di- and triphosphates.

By “CD39-mediated decrease in angiogenesis” we mean a decrease in the formation of blood vessels due to a decrease in CD39 expression, e.g., a decrease in transcription or translation, or a decrease in CD39 biological activity, e.g., a decrease in phosphohydrolysis of extracellular nucleoside di- and triphosphates.

By “CD39 transgene expressible genetic construct” we mean a construct containing a CD39 nucleic acid sequence that is positioned for expression. The construct can be inserted by artifice into a cell and can become a part of the genome of that cell and its progeny. Such a transgene may be partly or entirely heterologous to the cell.

The CD39 transgene may be in a construct that includes various types of promoters, e.g., a constitutive promoter or a regulatable promoter. The CD39 transgene may be in a viral vector, e.g., an adenovirus vector, a herpes virus vector, or a polio virus vector.

“Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, or in the case of in vitro assays, under conditions in which the assays are performed.

By “compound,” “test compound,” or “candidate compound” we mean any naturally occurring substance or synthetic chemical that is surveyed for its ability to decrease CD39 expression or activity, regulate platelet activation, modulate the P2 receptor signaling pathway, alter monocyte-macrophage or endothelial cell transmigration behavior, for example, by modulating expression of a gene, or a homolog of a gene, encoding a polypeptide or by modulating the function of a polypeptide, or fragment thereof. The term may refer to any medicinal substance used in humans or other animals. Encompassed within this definition are, for example, compound analogs, naturally occurring, synthetic and recombinant pharmaceuticals, hormones, or antibiotics.

By “control sample” we mean a test sample lacking a compound. Therefore, the control sample has all of the characteristics of the test sample except for the presence of a compound in the test sample.

By “decrease,” “inhibit,” or “block” we mean that a compound is able to reduce or completely prevent the expression of a gene encoding a polypeptide, or the biological activity of a polypeptide, that functions as an NTPDase, e.g., CD39, by at least 5%, more desirably, by at least 10%, even more desirably, by at least 25%, 50%, or 75%, and most desirably, by 90% or more as determined using the in vivo angiogenesis assay or the NTPDase assay provided in Example 1.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds.

By “increase,” “promote,” or “potentiate” we mean that a compound is able to enhance the expression of a gene encoding a polypeptide, or the biological activity of a polypeptide, that functions as an NTPDase, e.g., CD39, by at least 5%, more desirably, by at least 10%, even more desirably, by at least 25%, 50%, or 75%, and most desirably, by 90% or more as determined using the in vivo angiogenesis assay or the NTPDase assay provided in Example 1.

By “increased risk” is meant a greater than normal likelihood that a disease will occur in an individual. An increased risk of, for example, cancer or metastasis of cancer, rheumatoid arthritis, diabetic retinopathy, inflammatory bowel disease, or chronic radiation-induced proctitis is determined by detecting an increased level of CD39 mRNA or CD39 protein in a subject. An increased risk of, for example, peripheral vascular disease, cardiovascular disease, tissue organ engraftment rejection, or sequelae of ischemic reperfusion injury is determined by detecting a decreased level of CD39 mRNA or CD39 protein in a subject.

By “level of expression” or “expression” is meant the amount of transcription or translation of a specific gene, for example, the CD39 gene, which can be measured. A change in the level of expression may be determined, for example, for a polypeptide or nucleic acid molecule, and may be either an increase or a decrease relative to the level of a polypeptide or nucleic acid molecule under control conditions. The change in the level of expression is desirably an increase or decrease of at least 5%, 10%, 20%, 40%, 50%, 75%, 90%, 100%, 200%, 500%, or even 1000% as determined by methods known to those skilled in the relevant art.

Use of the term “a method of identifying” indicates that the method is appropriate for evaluating the effect of a number of compounds, often simultaneously. In this invention, such a method is directed to determining the ability of a compound to reduce angiogenesis.

By “modulating” is meant changing, by increase, decrease or otherwise. The change may be in amount, timing, or any other parameter. A decrease or increase in, for example, gene expression or activity, cell growth, cell transmigration, cell secretion of angiogenic factors, cell viability, receptor signaling, for example by the P2 receptor, and may be by at least 5%, more desirably at least 10%, even more desirably at least 25%, most desirably by 50% or more.

“Modulation” refers to the capacity to either promote or inhibit a functional property of a biological activity or process (e.g., enzyme activity or receptor signaling). Such enhancement or inhibition may be contingent on the occurrence of a specific event, such as activation of a signal transduction pathway, and/or may be manifest only in particular cell types.

The term “modulator” refers to a chemical (naturally occurring or non-naturally occurring), such as a biological macromolecule (i.e., nucleic acid, protein, non-peptide, or organic molecule), or an extract made from biological materials such as bacteria, plants, fungi, or animal (particularly mammalian) cells or tissues. Modulators are typically evaluated for potential activity as inhibitors or activators (directly or indirectly) of a biological process or processes (i.e., agonist, partial antagonist, partial agonist, antagonist, antineoplastic agents, cytotoxic agents, inhibitors of neoplastic transformation or cell proliferation, cell proliferation-promoting agents, and the like). The activity of a modulator may be known, unknown or partially known.

“Naturally-occurring” as used herein, as applied to an object, refers to the fact that an object can be found in nature. For example, a polypeptide or polynucleotide sequence that is present in an organism that can be isolated from a source in nature is naturally-occurring.

By “nucleotide analog” is meant a single base, e.g., adenine, thymidine, cytosine, or guanine, or a derivative of a single base that differs from the naturally-occurring form, in terms of chemical modifications known to those skilled in the art, and which share some or all properties of naturally-occurring forms. The nucleotide analog may contain one or more phosphate groups or may contain a modified phosphate group that is non-hydrolysable, e.g., ATPγS.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

By “peptide mimetic of CD39” we mean a molecule which serves as a substitute for a CD39 peptide in interactions with acceptor molecules (i.e., nucleosides (such as ATP and ADP); see Morgan et al., Ann. Reports Med. Chem. 24:243-252 (1989), for a review of peptide mimetics). A peptide mimetic of CD39, as used herein, includes synthetic molecules which may or may not contain amino acids and/or peptide bonds, but retain the structural and functional features of a peptide ligand. Those of skill in the art recognize that a variety of techniques are available for constructing a peptide mimetic with the same or similar desired biological activity as the corresponding peptide compound but with more favorable activity than the peptide with respect to solubility, stability, and susceptibility to hydrolysis and proteolysis. The term, “peptide mimetic” also includes peptoids and oligopeptoids, which are peptides or oligomers of N-substituted amino acids (Simon et al., Proc. Natl. Acad. Sci. USA 89:9367-9371 (1972)). Further included as peptide mimetics are peptide libraries, which are collections of peptides designed to be of a given amino acid length and representing all conceivable sequences of amino acids corresponding thereto.

By “pharmaceutically acceptable carrier” is meant a carrier which is physiologically acceptable to the treated mammal while retaining the therapeutic properties of the compound with which it is administered. One exemplary pharmaceutically acceptable carrier is physiological saline. Other physiologically acceptable carriers and their formulations are known to one skilled in the art and described, for example, in Remington 's Pharmaceutical Sciences, (18^{th} edition), ed. A. Gennaro, 1990, Mack Publishing Company, Easton, Pa. incorporated herein by reference.

By “polypeptide analog” is meant a fragment or derivative of an antigenic polypeptide that differs from naturally-occurring forms in terms of the identity or location of one or more amino acid residues (deletion analogs containing less than all of the residues specified for the polypeptide, substitution analogs wherein one or more residues specified are replaced by other residues and addition analogs where in one or more amino acid residues is added to a terminal or medial portion of the polypeptides) and which share some or all properties of naturally-occurring forms. These molecules include: the incorporation of codons “preferred” for expression by selected non-mammalian hosts; the provision of sites for cleavage by restriction endonuclease enzymes; and the provision of additional initial, terminal or intermediate DNA sequences that facilitate construction of readily expressed vectors.

By “polypeptide” is meant any chain of amino acids, regardless of length or post-translational modification (e.g., glycosylation or phosphorylation). By the use of “precursor” we mean that a polypeptide can be encoded by a full length gene sequence or by any portion of the coding sequence so long as the enzymatic activity is retained.

“Reporter enzyme activity” refers to the activity of a reporter enzyme in a membrane compartment and includes background reporter enzyme activity and de novo reporter enzyme activity. “Background reporter enzyme activity” refers to a reporter enzyme activity that exists in a membrane compartment that was not made in response to a stimulus, such as a test chemical. “De novo reporter enzyme activity” refers to a reporter enzyme activity that appears in a membrane compartment in response to a stimulus. De novo reporter enzyme activity is a reporter activity that is generated due to the synthesis of new reporter enzyme activity, or due to transcriptional events, such as differential splicing of RNA encoding a reporter enzyme activity leading to expression of reporter enzyme activity in response to a stimulus. Other sources of de novo reporter enzyme activity include, but are not limited to, co-translational modifications of reporter enzyme activity, post-translational modifications of reporter enzyme activity, change in location of reporter enzyme activity, conformational change of reporter enzyme activity, and other mechanisms that lead to appearance of a reporter enzyme activity in response to a stimulus. Post-translational modifications that may lead to de novo reporter enzyme activity include, but are not limited to, phosphorylation, dephosphorylation, oligosaccharide attachment or removal, signal peptide cleavage, pre-protein or pro-protein processing, myristylation, or farnesylation of the reporter enzyme activity. A background reporter enzyme activity and a de novo reporter enzyme activity can be the same enzyme activity, such as beta-lactamase activity. In such instances, background reporter enzyme activity can be referred to as “noise” and de novo reporter enzyme.

By “reporter gene,” we mean a gene that encodes a polypeptide that provides a detectable read-out, such as green flourescent protein or luciferase, that can be detected using methods known in the art, for example, optical methods such as absorbance or fluorescence detected microscopically or spectroscopically. The reporter gene can also encode a reporter enzyme, such as they are known in the art or are later developed, with specific enzymatic activity. The specific enzymatic activity can provide a detectable read-out, such as beta-lactamase, beta-galactosidase, or luciferase (for beta-lactamase, see WO 96/30540 to Tsien, published Oct. 3, 1996). Desirably, reporter enzymes localize in the cytosol of a cell, such as cytosolic beta-lactamase. Reporter enzymes can be detected using methods known in the art, such as the use of chromogenic or fluorogenic substrates for reporter enzymes as such substrates are known in the art. Such substrates are desirably membrane permeant. Chromogenic or fluorogenic readouts can be detected using, for example, optical methods such as absorbance or fluorescence. A reporter gene can be part of a reporter gene construct, such as a plasmid or viral vector, for example, a retrovirus or adeno-associated virus. A reporter gene can also be extra-chromosomal or it can be integrated into the genome of a host cell. The expression of the reporter gene can be under the control of exogenous expression control sequences or expression control sequences within the genome of the host cell. Under the latter configuration, the reporter gene is desirably integrated into the genome of the host cell.

By “sample” is meant a tissue biopsy, amniotic fluid, cell, blood, serum, urine, stool, or other specimen obtained from a patient or test subject. The sample can be analyzed to detect a mutation in a CD39 gene, or expression levels of a CD39 gene, by methods known in the art. For example, methods such as sequencing, single-strand conformational polymorphism (SSCP) analysis, or restriction fragment length polymorphism (RFLP) analysis of PCR products derived from a patient sample can be used to detect a mutation in a CD39 gene; ELISA can be used to measure levels of CD39 polypeptide; and PCR can be used to measure the level of a CD39 nucleic acid molecule.

By “specifically recognizes,” as used herein in reference to an antibody, is meant an increased affinity of an antibody for the protein against which it was raised, relative to an equal amount of any other protein. For example, an anti-CD39 antibody desirably has an affinity for CD39 that is least 2-fold, 5-fold, 10-fold, 30-fold, or 100-fold greater than for an equal amount of any other protein.

By “test sample” we mean one or more of the components of a sample and a compound.

By “therapeutically effective amount,” we mean the amount of CD39 polypeptide needed to produce a substantial clinical improvement. Optimal amounts will vary with the method of administration, and will generally be in accordance with the amounts of conventional medicaments administered in the same or a similar form.

By “treating, preventing, or stabilizing” is meant administering a therapeutic agent selected from the group consisting of: (a) a nucleotide analog, (b) a peptide, (c) an antibody, (d) an antisense nucleic acid or oligonucleotide, and (e) a compound, to modulate CD39 biological activity for the complete or partial recovery from a CD39-associated condition or symptoms associated with a CD39-associated condition, or for full recovery from a CD39-associated condition.

The invention provides several advantages. For example, it provides methods that can be used in the identification of therapeutic agents for the treatment, prevention, or stabilization, of a CD39-associated condition. These therapeutic agents can be selected from nucleotide analogs, peptides, antibodies, antisense oligonucleic acid molecules, or other compounds. These therapeutic agents can be used in methods to treat, prevent, or stabilize a CD39-associated condition using, for example, small molecule therapy, gene therapy, antisense oligonucleotide therapy, and protein replacement therapy. Additionally, the invention provides methods for the diagnosis and prognosis of diseases associated with CD39, for example, cancer or metastasis of cancer, rheumatoid arthritis, diabetic retinopathy, inflammatory bowel disease, tissue organ engraftment rejection, chronic radiation-induced proctitis, peripheral vascular disease (e.g., atherosclerosis, thromboembolic disease, or Buerger's disease (thromboangiitis obliterans)), cardiovascular disease (e.g., myocardial infarction, heart disease, or coronary artery disease), and sequelae of ischemic reperfusion injury.

### BRIEF DESCRIPTION OF THE DRAWINGS

FIGS. 1A, 1B, 1C, and 1D are photographs of sections from MATRIGEL® plugs in wild-type (1A, 1C) and cd39-null (1B, 1D) mice, stained with antibodies recognizing PECAM-1/CD31, an endothelial and platelet marker. (1A through 1D). Brackets denote MATRIGEL®. FIG. 1A and 1C: PECAM-1-positive vessels infiltrating MATRIGEL® in wild-type mice (arrow). FIGS. 1B and 1D: lack of infiltration of PECAM-1-positive vessels into MATRIGEL® in cd39-null mice (arrow). Bars=25 μm (1C and 1D) and 200 μm (1A and 1B).

FIGS. 2A, 2B, 2C, 2D, 2E, and 2F are photographs of sections from MATRIGEL® plugs in wild-type (2A, 2C, and 2E) and cd39-null (2B, 2D, and 2F) mice stained with Hematoxylin-eosin (H&E)(2A and 2B) or antibodies recognizing F4/80 (2C through 2F), a monocyte/macrophage marker. FIG. 2A: H&E staining depicting the interface between dermis and MATRIGEL® in wild-type mice. FIG. 2B: H&E staining depicting the interface between dermis and MATRIGEL® in cd39-null mice. Note the lack of cells within the MATRIGEL® itself. FIG. 2C and 2E: Marked F4/80-positive macrophage infiltration into MATRIGEL® plug in wild-type animals (arrows). FIG. 2D and 2F: Compacted rim of F4/80-positive mutant monocyte/macrophages at dermis/MATRIGEL® interface (arrows). Note the absence of monocyte/macrophage infiltration into MATRIGEL®. Bars=25 μm (2A, 2B, 2E, and 2F) and 200 μm (2C and 2D).

FIGS. 3A, 3B, 3C, and 3D are photographs of fluorescently stained MATRIGEL® plugs. Double immunofluorescent staining (3A through 3D) of sections from MATRIGEL® in wild-type (3A and 3C) and cd39-null (3B and 3D) mice with antibodies recognizing F4/80-positive macrophages (3A through 3D), cd39/PECAM-1-positive endothelial cells (3A and 3B), and NG2-positive pericytes (3C and 3D). M indicates MATRIGEL®. FIG. 3A: infiltrating F4/80-positive monocyte/macrophages (green) in close proximity to infiltrating PECAM-1-positive endothelial cells (red) in MATRIGEL® plugs from wild-type mice. FIG. 3B: F4/80-positive monocyte/macrophage rim (green) corresponding to inflammatory zone in dermis at the dermis/MATRIGEL® interface followed by a zone of PECAM-1-positive endothelium constituting vascular zone (red) in mutant mice. FIG. 3C: F4/80-positive monocyte/macrophages (green) infiltrating along a connective tissue septum into MATRIGEL® followed by NG2-positive pericytes (red) in wild-type mice. FIG. 3D: F4/80-positive monocyte/macrophage rim at dermis/MATRIGEL® interface (green). NG2-positive pericytes (also stained positive for smooth muscle actin and PDGF-b receptors) appear unable to traverse other cell populations and then migrate into vascular zone. Bars=100 μm.

FIGS. 4A, 4B, 4C, 4D, 4E, 4F, 4G, 4H, and 4I are graphs showing the cytofluorometric analysis of monocyte/macrophages. Surface densities of αₘβ₂ were measured by fluorescence activated cell sorter (FACS®). FIGS. 4A-4C: isospecific IgG-matched negative controls (FITC and phycoerythrin; FIG. 4C) adjacent to gating parameters (FIGS. 4A and 4C). Median levels of wild-type macrophage cell surface coexpression of αₘβ₂ (MAC-1) and ICAM-1 increased significantly (n=6; P<0.05) compared to the control (FIG. 4D) after short-term stimulation with 200 μmol/L ATP (FIG. 4E) or 200 μmol/L ATPγS (FIG. 4F)(30 minutes; n=6). In contrast, αₘβ₂ (MAC-1) and ICAM-1 coexpression levels in quiescent cd39-null macrophages were largely unaltered compared to the control (FIG. 4G) after stimulation with 200 μmol/L ATP (FIG. 4H) or 200 μmol/L ATPγS (FIG. 41).

FIG. 5 is a graph showing indexed amounts of transmigration of monocyte/macrophages through MATRIGEL® in the presence of the indicated agonists macromolecule(s). Stimulation with ATP and serotonin alone had no substantial effect on migration of cd39-null macrophages. ATP in combination with serotonin, however, significantly boosted migration of both control wildtype (WT) and cd39-null knockout (KO) macrophages.

FIGS. 6A-6F are photographs showing the development of subperitoneal tumors (B16-F10) in a representative control wildtype (WT) mouse (FIGS. 6A-6C) or a cd39-null knockout (KO) mouse (FIGS. 6D-6F). The subperitoneal tumor is located within the abdominal wall flap. Tumor cells (B16-F10; 5.0×10⁵ cells in 0.1 ml PBS) were injected into the subperitoneal space and examined after injection (FIGS. 6B and 6E) and after seven days (FIG. 6C and 6F) in n=5 WT and KO mice. Tumor sizes in the WT mouse (FIG. 6C) are clearly larger than the tumor in the KO (FIG. 6F) mouse. The tumor in the WT mouse also exhibits obvious neovascularization, absent in the mutant KO mouse.

FIG. 7 is a graph showing the evaluation of new vasculature with the growth of B16-F10 tumor cells in a representatve mouse from WT mouse, a cd39 heterozygote, or a cd39 knockout group (n=2 per group). The inner aspect of the abdominal flap was examined at 2-day intervals under anesthesia to measure tumor size, calculate volume and quantify newly formed blood vessels adjacent to tumor by microscopy (one heterozygote mouse died at day 1).

FIGS. 8A and 8B are photographs showing development of a subcutaneous tumor (B16-F10) in mice. FIG. 8A is a comparison of tumor growth in a WT mouse (+/+) (n=8)and a cd39 heterozygote (±)(n=6). FIG. 8B is a comparison of B16-F10 tumor growth in a WT mouse and a cd39 heterozygote (±). FIG. 8C is a comparison of tumor growth in a WT mouse and a cd39 knockout mouse (−/−)(n=6). 3.0×10⁶ cells of B16-F10 in 0.1 ml PBS were injected in the dorsal subcutaneous tissue. (Tumor bearing mice were killed 15 days after tumor cell injection.)

FIG. 9 is a photograph showing development of a subcutaneous tumor (LLC) in a WT mouse, a cd39 heterozygote, and a cd39 knockout mouse. 3.0×10⁶ cells of LLC in 0.1 ml PBS were injected in the dorsal subcutaneous space. Tumor bearing mice were killed 15 days after tumor cell injection.

FIG. 10 is a graph showing the growth rate of subcutaneously transplanted B16-F10 tumor cells in mice. 3.0×10⁶ cells of B16-F10 in 0.1 ml PBS were injected in the dorsal subcutaneous space. Tumor bearing mice were killed 15 days after tumor cell injection. Tumors were measured in two dimensions every day and volume was calculated as V=L·W²·π/6 (V: volume, L: longest diameter, W: shortest diameter, π: the circular constant)(WT n=8, heterozygote n=6 and KO, n=6).

FIG. 11 is a graph showing the growth rate of subcutaneously transplanted LLC tumor cells in mice. 3.0×10⁶ cells of LLC in 0.1 ml PBS were injected in the dorsal subcutaneous space. Tumor bearing mice were killed 15 days after tumor cell injection. Tumors were measured in two dimensions every day and volume was calculated as V=L·W²·π/6 (V: volume, L: longest diameter, W: shortest diameter, π: the circular constant).

FIG. 12 is a photograph showing pulmonary metastases in the lungs of a representative WT mouse, a cd39 heterozygote, and a cd39 knockout mouse. Tumor cells (1.5×10⁵ cells B16-F10 in 0.1 ml PBS per 25 grams body weight) were injected into systemic circulation via the inferior vena cava using a 26-gauge needle. The mice were observed daily. Tumor bearing mice were killed 15 days after tumor cell injection and the lungs were harvested for evaluation of tumor size and number. Obvious increases in metastasis and lung size were observed in WT mice (WT n=5)(KO n=5).

FIG. 13 is a photograph showing pulmonary metastases in the lungs of a representative WT mouse and a cd39 heterozygote injected with tumor cells. LLC tumor cells were injected into systemic circulation via the inferior vena-cava. The mice were observed daily. Tumor bearing mice were killed 15 days after tumor cell injection and the lungs were harvested for evaluation of tumor size and number. Obvious increases in metastasis and lung size were observed in WT mice (WT n=5) (Heterozygote n=5).

FIGS. 14A-14C are photographs of rectosigmoid biopsies taken from the rectum of a control subject (FIG. 14A) and from the sigmoid colon (FIG. 14B) and rectum (FIG. 14C) of a subject who had previously received radiation treatment and gone onto develop radiation proctitis. The biopsies have been stained with Hematoxylin-eosin (H&E). Radiation proctitis, which results in fibrosis, inflammation, and abnormal vasculature, is observed in the rectal biopsy (FIG. 14C) due to damage caused by the radiation treatment.

FIGS. 15A-15C are photographs of rectosigmoid biopsies that have been stained with antibodies recognizing PECAM-1/CD31, an endothelial cell marker. FIG. 15A is a photograph of a biopsy taken from the rectum of a control subject. FIGS. 15B and 15C are photographs of biopsies taken from the sigmoid colon (FIG. 15B) and rectum (FIG. 15C) of a subject post-radiation treatment. FIG. 15C: The PECAM-1-stains abnormal microvessels in the rectal biopsies that exhibit irregular size and characteristics.

FIGS. 16A and 16B are photographs of biopsies taken from the sigmoid colon (FIG. 16A) and the colon (FIG. 16B) of a patient post-radiation treatment. The biopsies have been stained with Hematoxylin-eosin. FIG. 16B: Radiation treatment results in the vascular proliferation of small vessels when the endothelium expresses P2Y2 receptor nucleotide g-protein coupled receptor of activated endothelium.

FIGS. 17A-17C are photomicrographs of rectosigmoid biopsies stained with antibodies to the vitrorectum receptor (B3 integrim). This integrim receptor is crucial for angiogenesis. FIG. 17A is a photograph of biopsies taken from the rectum of a control subject. FIGS. 17B and 17C are photographs of biopsies taken from the sigmoid colon (FIG. 17B) and rectum (FIG. 17C) of a subject post-radiation treatment. The immunostaining reveals proliferating, small vessels in the irradiated rectum that stain heavily for the β3 chain of the vitrorectum receptor.

FIGS. 18A-18C are photomicrographs of rectosigmoid biopsies stained with antibodies to the CD39, the dominant ectonucleotidase of the vasculature and macrophages. FIGS. 18B and 18C are photographs of biopsies taken from the sigmoid colon (FIG. 18B) and rectum (FIG. 18C) of a subject post-radiation treatment. The immunostaining reveals high level expression of CD39 in macrophages and proliferating microvessels in the irradiated rectal biopsy when compared to the rectal biopsies of controls and unrestricted non-irradiated sigmoid colon.

FIG. 19 is a photograph showing heightened CD39 immunoreactivity in a rectal biopsy taken from a patient with chronic radiation proctitis. Heightened CD39 immunoreactivity in this high power view is associated with the vasculature and infiltrating macrophages. The high level expression in the vasculature appears associated with vascular proliferation and would also predispose to a localized bleeding tendency given the anti-platelet effects of CD39.

FIG. 20 is a graph showing NTPDase activity in spleen (control) versus the tumor cell lines B16-F10 and LLC. Both ATP and ADP are equally catalyzed by the spleen, however the B16-F10 cell line has low level ATP and ADPase activity; whereas the Lewis Lung Cancer (LLC) line has preferential ATPase activity.

FIG. 21 is a photograph of a Western blot showing the expression of CD39 in the lung and spleen of a wild type mouse. The homogenate and particulate fractions of mouse (wildtype) lung were subjected to Western blotting with antibodies to mouse CD39. The typical 78 kd dominant band of CD39 is observed. No CD39 is expressed by either B16-F10 or LLC lines. CD39 expression is absent in the B16-F10 and LLC tumor cell lines. CD39 is detected using a rabbit polyclonal antibody to mouse CD39.

FIGS. 22A-22C are photographs showing the representative impairment of tumor angiogenesis in cd39 null mice. FIG. 22A is a photograph of a section of B16-F10 taken from a wild-type cd39 mouse that has been stained with antibodies that recognize PECAM-1/CD31. PECAM-1-positive vessels infiltrate the tumor in wild-type mouse. In contrast, FIGS. 22B and 22C show that vessels (stained for PECAM-1/CD3 1) do not infiltrate the tumor site in cd39 null mice, but remain at the tumor/host interface.

FIGS. 23A and 23B are photographs showing the impairment of tumor angiogenesis in cd39 null mice. FIG. 23A is a photograph of a section taken from a wild-type cd39 mouse that has been stained for heparan sulfate proteoglycan (HSPG). PECAM-1-positive vessels infiltrate the tumor in wild-type mice. In contrast, FIG. 23B shows that vessels do not infiltrate the tumor site in cd39 null mice.

FIGS. 24A-24D are photographs showing the effect of the deletion of CD39 in the host on vessel formation during implanted tumor growth. FIGS. 24A and 24B are photographs of sections taken from wild type mice at the site of tumor formation. The sections are stained for (HSPG) and show abundant vessel formation into the tumor in the presence of CD39. FIGS. 24C and 24D are photographs of sections taken from cd39 null mice at the site of tumor formation. In contrast to the wild type sections, no formation of vessels or basement membrane occurs in the knock out mice.

FIGS. 25A-25F are photographs showing the typical CD39 expression in the vasculature of porcine liver tissue (FIGS. 25A-25C), porcine kidney tissue (FIG. 25F), human liver tissue (FIG. 25D), and murine liver tissue (FIG. 25E). The major expression of CD39 in quiescent tissues is the endothelium and smooth muscle of hepatic arterioles, portal vein branches, periductular vascular plexus, and the central vein elements. Within the hepatic sinusoids, only Kupffer cells stain for CD39. The fenestrated sinusoidal endothelium is negative.

FIG. 26 is a graph showing liver regeneration following post 70% hepatectomy in wild type and cd39 knock out mice. Although liver regeneration in both the wild type and the cd39 knock out mice appears equivalent, only 50% of KO mice (n=5 per group) survive for 21 days. The liver regeneration index is a measure of live/body weight ratios over time.

FIGS. 27A-27D are photographs of terminal deoxynucleotidyl transferase (TdT)/TUNEL-stained (brown) and Hematoxylin-eosin-stained (blue) sections showing liver cell death at day 14 post hepatectomy. FIG. 27A is from a wild type mouse. FIGS. 27B-27D are sections removed from cd39 null mice.

FIGS. 28A-28D are photographs showing the expression of CD39 in murine hepatic sinusoids following hepatectomy in wild type mice. FIG. 28A is a control section taken prior to 70% hepatectomy. FIGS. 28B, 28C, and 28D are photographs taken at day 2, day 7, and day 14, respectively, following hepatectomy. CD39 is upregulated on sinusoidal endothelium undergoing proliferation post-hepatectomy.

FIGS. 29A-29D are photographs showing the failure of CD31+ vascular endothelial cells to grow into hepatic sinusoids following hepatecto my in cd39 null mice. FIGS. 28A and 28B are photographs showing growth of CD31+ vascular endothelial cells in CD39 positive wild type mice at day 2 and day 7, respectively, post hepatectomy. In contrast, FIGS. 29C and 29D show failure of growth of CD31+ vascular endothelial cells into hepatic sinusoidal cd39 null mice at day 2 and day 7, respectively. This alteration results in the hypovascular islands of hepatocytes post-proliferation that in turn undergo apoptosis (depicted in FIGS. 27B-D).

FIG. 30 is a graph showing the differential kinetics of hepatocyte proliferation and angiogenesis following 70% hepatectomy in cd39 null mice. In wild type mice (+/+), hepatocyte and endothelial cell proliferation is observed (as indicated by BrdU incorporation). cd39 null mice (−/−) exhibit moderate hepatocyte cell growth, but lack endothelial cell growth. A striking increase in endothelial cell apoptosis is also observed in cd39 null mice at the time points associated with hepatocyte injury.

FIGS. 31A and 31B are photomicrographs showing differential infiltration of MMP9 monocytes and disordered angiogenesis in cd39 null mice. FIGS. 31A and 31B are photographs of biopsies taken from wild type hearts (FIG. 31A) and ischemic and myocardial tissues from the cd39 null mice (FIG. 31B). In contrast to the wild type hearts, low levels of MMP9 expressing cells were observed by immunoperoxidase staining in ischemic and necrotic myocardial tissues in the cd39 null mice.

FIG. 32 is a photograph showing myocardial rupture in wild type mice following LAD ligation. This event resulted in 40% mortality in wild type mice at day 7, whereas cd39 null mice appeared to be protected against myocardial rupture and the associated cellular infiltration and angiogenic responses (10/10 surviving to day 7).

FIGS. 33A and 33B are graphs showing the abnormal hepatic endothelial cell (EC) responses to regeneration stress in cd39 null mice. FIG. 33A: BrdU positive ECs were counted over time in the model of post-70% hepatectomy. Minimal proliferative responses were seen in cd39 null mice, relative to wild type (n=10). FIG. 33B: Heightened levels of Tunel positive apoptotic ECs were observed by days 5-7 in the same groups of cd39 null mice, resulting in profound derangements in liver replacement and islands of ischemic injury.

FIG. 34 is a photograph depicting the effects of ATP stimulation on integrin-mediated signaling, as determined using Western blot analysis. Both wild type and cd39 null vascular cells were studied under basal and nucleotide-stimulated conditions. Parallel experiments were performed using anti-CD39 ligation. Cellular extracts were prepared 2 hours post-ATP stimulation (100 μM) and subjected to Western blotting using an anti-p27, an anti-cdk2, an anti-cdk4, an anti-Rb, and an anti-β actin antibody.

### DETAILED DESCRIPTION

We provide evidence below that CD39 plays a crucial role in modulating angiogenesis and that controlling CD39 biological activity allows therapeutic management of angiogenesis.

Nucleotide triphosphates activate P2 receptors on monocyte, macrophage, and endothelial cells and promote their adhesion and migration to sites of neovascularization. CD39 regulates the cellular infiltration of monocytes, macrophages, and endothelial cells to areas of blood vessel formation by converting NTP into NDP or NMP, which is then converted to the nucleoside analog, thus reducing the local concentration of NTP and NDP and preventing activation of the P2 receptor. We have found that the absence of CD39 increases the local concentration of NTP and causes the P2 receptor to be desensitized and downregulated, thereby preventing the initiation of angiogenesis and progression of neovascularization. Accordingly, we conclude that CD39 regulates angiogenesis by controlling the local concentration of nucleotide triphosphates, which, in turn, regulate the activation of P2 receptors, and hence, adhesion of monocytes, macrophages, and endothelial cells to matrix components and other cells.

We demonstrate that CD39 plays a role in the process of angiogenesis using a cd39 null mouse model and an in vivo MATRIGEL® assay, a well accepted model for angiogenesis. This assay has certain advantages over other in vivo models, such as wound healing and tumor formation. The evaluation of reconstituted basement membrane matrices incorporating peptide growth hormones and other factors has facilitated evaluation of endothelial cell requirements for formation of capillary networks in vivo. Specifically, platelet derived sphingosine 1-phosphate (SPP; Lee et al., Cell 99:301 (1999)) appears to be an important regulator of angiogenesis.

cd39 null mice implanted with either MATRIGEL® plugs containing growth factors or tumor cells exhibited lower vessel density compared to wild-type mice. Therefore, the results, presented below, indicate that a method that decreases the activity of CD39 will decrease angiogenesis. Conditions involving angiogenesis to which this is relevant include cancer or metastasis of cancer, chronic inflammatory illnesses (e.g., rheumatoid arthritis), inflammatory bowel disease, diabetic retinopathy, and chronic radiation-induced proctitis, all of which involve inappropriate angiogenesis. Our findings also allow enhancement of angiogenesis, where desirable, such as in the case of peripheral vascular disease (e.g., atherosclerosis, thromboembolic disease, and Buerger's disease (thromboangiitis obliterans)), diseases related to cardiovascular disease (e.g., myocardial infarction, heart disease, or coronary artery disease), tissue organ engraftment rejection, and sequelae of ischemic reperfusion injury.

Compounds that modulate CD39 biological activity are known. In addition, new compounds that modulate CD39 biological activity, and therefore affect angiogenesis, can also be identified.

Identification of a Candidate Compound that Modulates CD39 Biological Activity

The identification of a candidate compound that can act specifically to modulate CD39 biological activity, and thereby potentiate or block angiogenesis, can be identified by the methods of the present invention. The candidate compound is identified for its usefulness in the treatment, stabilization, or prevention of an angiogenesis-associated conditions. A candidate compound can be identified by its ability to modulate (e.g., potentiate or block) either the biological activity of a CD39 polypeptide or the expression level of a CD39 gene.

Compounds that are identified by the methods of the present invention that decrease the biological activity or expression levels of a CD39 polypeptide, represent compounds for the treatment, stabilization, or prevention of conditions exacerbated by angiogenesis, such as cancer or metastasis of cancer, chronic inflammatory illnesses (e.g., rheumatoid arthritis), inflammatory bowel disease, diabetic retinopathy, or chronic radiation-induced proctitis. A candidate compound identified by the present invention can decrease the biological activity of a CD39 polypeptide, for example, by decreasing the phosphohydrolysis of nucleoside di- and triphosphates. A candidate compound identified by the methods of the present invention can also, for example, decrease the expression of a CD39 gene by decreasing transcription of the CD39 gene, or translation or stability of the CD39 mRNA.

Compounds that are identified by the methods of the present invention that increase the biological activity or expression levels of a CD39 polypeptide, represent compounds for the treatment, stabilization, or prevention of a disease or diseases associated with insufficient angiogenesis, for example, peripheral vascular disease, cardiovascular disease, thromboembolic disease, tissue organ engraftment rejection, and sequelae of ischemic reperfusion injury. A candidate compound identified by the present invention can increase the biological activity of a CD39 polypeptide, for example, by increasing the phosphohydrolysis of nucleoside di- and triphosphates. A candidate compound identified by the methods of the present invention can also, for example, increase the expression of a CD39 gene by increasing transcription of the CD39 gene, or translation or stability of the CD39 mRNA.

Desirable candidate compounds can be selected from, among others, (a) nucleotide analogs, (b) peptides (for example, expression of CD39, see Gangadharan et al., Surgery 130: 296-303 (2001), incorporated herein fully by reference), (c) antibodies, (d) antisense or oligonucleotide analogs (see, Imai et al., Biochemistry 38: 13473-13479 (1999), incorporated herein fully by reference), and (e) natural and synthetic compounds. Other molecules that modulate CD39 biological activity, such as molecules related to the suramin-Evans blue families, can also be identified by the methods of the invention.

Nucleotide Analogs

Nucleotide analogs that modulate the biological activity of CD39 can also be identified by the methods of the invention. Desirably, nucleotide analogs identified by the methods of the invention, would bind to CD39 and prevent enzymatic function, i.e., phosphohydrolysis. An ideal CD39 nucleotide analog inhibitor should not be a P2 receptor agonist and should not be dephosphorylated by the enzyme. One problem encountered with analogs of ATP is their rapid dephosphorylation, thereby masking the effects on P2 receptors. This problem can be partially circumvented by the use of non-hydrolysable ATP analogs.

Depending on the desired outcome, a nucleotide analog of the invention could be identified that specifically interacts with P2 receptors and acts as a P2 receptor agonist/antagonist.

Peptides

The isolation of peptides that are capable of binding to specific polypeptides, for example CD39, in such a way to modulate biological activity is fundamental to discovering new therapeutics. The ability to synthesize DNA chemically has made possible the construction of extremely large collections of nucleic acid and peptide sequences as potential ligands. Recently developed methods allow efficient screening of libraries for desired binding activities (see Pluckthun & Ge, Angew. Chem. Int. Ed. Engl. 30, 296-298 (1991). For example, RNA molecules with the ability to bind a particular protein (see Tuerk & Gold, Science 249, 505-510 (1990) or a dye (see Ellington & Szostak, Nature 346, 818-822 (1990) have been selected by alternate rounds of affinity selection and PCR amplification. A similar technique was used to determine the DNA sequences that bound a human transcription factor (see Thiesen & Bach, Nucl. Acids Res. 18, 3203-3209 (1990)). Peptides, screened by phage display techniques for the intrinsic ability to interact with CD39, can be tested by the methods of the invention for the ability to modulate CD39 biological activity, e.g., the ability to promote or inhibit the phosphohydrolysis of nucleoside di- and triphosphates.

Antibodies

Antibody-based compounds of the invention can also be identified and include function-blocking antibodies targeted to the active site of CD39, for example, antibodies that block the phosphohydrolytic activity of CD39, or antibodies that inhibit nucleotide P2 receptor signaling.

To prepare polyclonal antibodies, CD39 proteins, fragments of CD39 proteins, or fusion proteins containing defined portions of CD39 proteins can be synthesized in, e.g., bacteria by expression of corresponding DNA sequences contained in a suitable cloning vehicle. Fusion proteins are commonly used as a source of antigen for producing antibodies. Two widely used expression systems for E. coli are lacZ fusions using the pUR series of vectors and trpE fusions using the pATH vectors. The proteins can be purified, and then coupled to a carrier protein, mixed with Freund's adjuvant (to enhance stimulation of the antigenic response in an inoculated animal), and injected into rabbits or other laboratory animals. Alternatively, protein can be isolated from CD39-expressing cultured cells. Following booster injections at bi-weekly intervals, the rabbits or other laboratory animals are then bled and the sera isolated. The sera can be used directly or can be purified prior to use by various methods, including affinity chromatography employing reagents such as Protein A-Sepharose, antigen-Sepharose, and anti-mouse-Ig-Sepharose. The sera can then be used to probe protein extracts from CD39-expressing tissue fractionated by polyacrylamide gel electrophoresis to identify CD39 proteins. Alternatively, synthetic peptides can be made that correspond to antigenic portions of the protein and used to inoculate the animals.

To generate peptide or full-length protein for use in making, for example, CD39-specific antibodies, a CD39 coding sequence can be expressed as a C-terminal or N-terminal fusion with glutathione S-transferase (GST; Smith et al., Gene 67:31-40 (1988)). The fusion protein can be purified on glutathione-Sepharose beads, eluted with glutathione, cleaved with a protease, such as thrombin or Factor-Xa (at the engineered cleavage site), and purified to the degree required to successfully immunize rabbits. Primary immunizations can be carried out with Freund's complete adjuvant and subsequent immunizations performed with Freund's incomplete adjuvant. Antibody titers can be monitored by Western blot and immunoprecipitation analyses using the protease-cleaved CD39 fragment of the GST-CD39 fusion protein. Immune sera can be affinity purified using CNBr-Sepharose-coupled CD39 protein. Antiserum specificity can be determined using a panel of unrelated GST fusion proteins.

Alternatively, monoclonal CD39 antibodies can be produced by using, as an antigen, CD39 protein isolated from CD39-expressing cultured cells or CD39 protein isolated from tissues. The cell extracts, or recombinant protein extracts containing CD39 protein, can, for example, be injected with Freund's adjuvant into mice. Several days after being injected, the mouse spleens can be removed, the tissues disaggregated, and the spleen cells suspended in phosphate buffered saline (PBS). The spleen cells serve as a source of lymphocytes, some of which would be producing antibody of the appropriate specificity. These can then be fused with permanently growing myeloma partner cells, and the products of the fusion plated into a number of tissue culture wells in the presence of selective agents, such as hypoxanthine, aminopterine, and thymidine (HAT). The wells can then be screened by ELISA to identify those containing cells making antibody capable of binding to a CD39 protein, polypeptide fragment, or mutant thereof. These cells can then be re-plated and, after a period of growth, the wells containing these cells can be screened again to identify antibody-producing cells. Several cloning procedures can be carried out until over 90% of the wells contain single clones that are positive for specific antibody production. From this procedure, a stable line of clones that produce the antibody can be established. The monoclonal antibody can then be purified by affinity chromatography using Protein A Sepharose and ion-exchange chromatography, as well as variations and combinations of these techniques. Once produced, monoclonal antibodies are also tested for specific CD39 protein recognition by Western blot or immunoprecipitation analysis (see, e.g., Kohler et al., Nature 256:495, 1975; Kohler et al., European Journal of Immunology 6:511, 1976; Kohler et al., European Journal of Immunology 6:292, 1976; Hammerling et al., In Monoclonal Antibodies and T Cell Hybridomas, Elsevier, New York, N.Y., 1981; Ausubel et al., supra).

As an alternate or adjunct immunogen to GST fusion proteins, peptides corresponding to relatively unique hydrophilic regions of CD39 can be generated and coupled to keyhole limpet hemocyanin (KLH) through an introduced C-terminal lysine. Antiserum to each of these peptides can be similarly affinity-purified on peptides conjugated to BSA, and specificity tested by ELISA and Western blotting using peptide conjugates, and by Western blotting and immunoprecipitation using CD39, for example, expressed as a GST fusion protein.

Antibodies of the invention can be produced using CD39 amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson et al., CABIOS 4:181, 1988. These fragments can be generated by standard techniques, e.g., by the PCR, and cloned into the pGEX expression vector. GST fusion proteins can be expressed in E. coli and purified using a glutathione-agarose affinity matrix (Ausubel et al., supra). To generate rabbit polyclonal antibodies, and to minimize the potential for obtaining antisera that is non-specific, or exhibits low-affinity binding to a CD39 protein, two or three fusions are generated for each protein, and each fusion is injected into at least two rabbits. Antisera are raised by injections in series, preferably including at least three booster injections.

In addition to intact monoclonal and polyclonal anti-CD39 antibodies, the invention features various genetically engineered antibodies, humanized antibodies, and antibody fragments, including F(ab′)2, Fab′, Fab, Fv, and sFv fragments. Truncated versions of monoclonal antibodies, for example, can be produced by recombinant methods in which plasmids are generated that express the desired monoclonal antibody fragment(s) in a suitable host. Antibodies can be humanized by methods known in the art, e.g., monoclonal antibodies with a desired binding specificity can be commercially humanized (Scotgene, Scotland; Oxford Molecular, Palo Alto, Calif.). Fully human antibodies, such as those expressed in transgenic animals, are also included in the invention (Green et al., Nature Genetics 7:13-21, 1994).

Ladner (U.S. Pat. Nos. 4,946,778 and 4,704,692) describes methods for preparing single polypeptide chain antibodies. Ward et al., Nature 341:544-546, 1989, describes the preparation of heavy chain variable domains, which they term “single domain antibodies,” and which have high antigen-binding affinities. McCafferty et al., Nature 348:552-554, 1990, show that complete antibody V domains can be displayed on the surface of fd bacteriophage, that the phage bind specifically to antigen, and that rare phage (one in a million) can be isolated after affinity chromatography. Boss et al., U.S. Pat. No.4,816,397, describes various methods for producing immunoglobulins, and immunologically functional fragments thereof, that include at least the variable domains of the heavy and light chains in a single host cell. Cabilly et al., U.S. Pat. No. 4,816,567, describes methods for preparing chimeric antibodies.

Antisense or Oligonucleotide Analogs

Antisense compounds are commonly used as research reagents and diagnostics. For example, antisense oligonucleotides, which are able to inhibit gene expression with exquisite specificity, are often used by those of ordinary skill to elucidate the function of particular genes, and, in many cases, their relevance to disease processes. This is often referred to as “target validation.” Antisense compounds are also used, for example, to distinguish between functions of various members of a biological pathway. Antisense modulation has, therefore, been harnessed for research use. Antisense compounds can be used as therapeutic agents as well. The administration of antisense compounds would provide a means to inhibit gene expression in a patient in need thereof. These compounds may include CD39 antisense RNA, dsRNA (containing in whole or in part the CD39 gene sequence), or any other effective nucleic acid-based compound known to be useful for decreasing gene transcription, translation, or expression by those of skill in the art.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases. Particularly preferred are antisense oligonucleotides comprising from about 8 to about 30 nucleobases (i.e., from about 8 to about 30 linked nucleosides).

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalklyphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above type of phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,625,050; and 5,697,248, each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts.

Representative United States patents that teach the preparation of the above type of oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone.

Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 254, 1497 (1991).

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O—, S—, or N-alkyl, O—, S—, or N-alkenyl, or O—, S— or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′—O—CH₂CH₂OCH₃, also known as 2′—O—(2-methoxyethyl) or 2′-MOE) [Martin et al., Helv. Chim. Acta, 78, 486 (1995)] i.e., an alkoxyalkoxy group. A further preferred modification includes 2′-dimethylaminooxyethoxy, i.e., a 2′-O(CH₂)₂ ON(CH₃)₂ group, also known as 2′-DMAOE, as described in U.S. Pat. No. 6,127,533, the contents of which are herein incorporated by reference.

Other preferred modifications include 2′-methoxy (2′—O—CH₃), 2′-aminopropoxy (2′—OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Representative United States patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,0531 5,639,873; 5,646,265; 5,658,873; 5,670,633; and 5,700,920, each of which is herein incorporated by reference.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C or m5c), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, those disclosed by Englisch et al., Angewandte Chemie, International Edition, 30, 613 (1991), and those disclosed by Sanghvi, Y. S., Chapter 15, Antisense Research and Applications, pages 289-302, Crooke, S. T. and Lebleu, B., ed., CRC Press, 3. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808., as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, each of which is herein incorporated by reference.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA 86:6553 (1989)), cholic acid (Manoharan et al., Bioorg. Med. Chem. Lett. 4:1053 (1994)), a thioether, e.g., hexyl-S-tritylthiol (Manoharan et al., Ann. N. Y Acad. Sci. 660:306 (1992); Manoharan et al., Bioorg. Med. Chem. Let. 3:2765 (1993)), a thiocholesterol (Oberhauser et al., Nucl. Acids Res. 20:533 (1992)), an aliphatic chain, e.g., dodecandiol or undecyl residues (Saison-Behmoaras et al., EMBO J. 10:111 (1991); Kabanov et al., FEBS Lett. 259:327 (1990); Svinarchuk et al., Biochimie 75:49 (1993)), a phospholipid, e.g., dihexadecyl-rac-glycerol or triethylammonium 1,2-di-O-hexadecyl-rac-glycero-3-H-phosphonate (Manoharan et al., Tetrahedron Lett. 36:3651 (1995); Shea et al., Nucl. Acids Res. 18 (1990)), a polyamine or a polyethylene glycol chain (Manoharan et al., Nucleosides & Nucleotides 14:969 (1995)), or adamantane acetic acid (Manoharan et al., Tetrahedron Lett. 36:3651 (1995)), a palmityl moiety (Mishra et al., Biochim. Biophys. Acta 1264:229 (1995)), or an octadecylamine or hexylamino-carbonyl-oxycholesterol moiety (Crooke et al., J. Pharmacol. Exp. Ther. 277:923(1996)).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the present invention can be utilized for diagnostics, therapeutics, prophylaxis and as research reagents and kits. For therapeutics, an animal, preferably a human, suspected of having a disease or disorder involving angiogenesis, or the lack thereof, which can be treated by modulating the expression of CD39 is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent tumor formation.

The antisense compounds of the invention are useful for research and diagnostics, because these compounds hybridize to nucleic acids encoding CD39, enabling sandwich and other assays to easily be constructed to exploit this fact. Hybridization of the antisense oligonucleotides of the invention with a nucleic acid encoding CD39 can be detected by means known in the art. Such means may include conjugation of an enzyme to the oligonucleotide, radiolabeling of the oligonucleotide or any other suitable detection means. Kits using such detection means for detecting the level of CD39 in a sample may also be prepared.

Compounds

In general, novel drugs that modulate CD39 biological activity, i.e., to increase or decrease angiogenesis, are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, for example, from Brandon Associates (Merrimack, N.H.), Aldrich Chemical (Milwaukee, Wis.), and Sigma-Aldrich (St. Louis, Mo.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods, and, if desired, any library or compound can be readily modified using standard chemical, physical, or biochemical methods. Furthermore, antibodies (or fragments thereof) may also be generated using standard techniques known in the art and screened for their efficacy in modulating or reducing angiogenesis using the techniques described herein.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their therapeutic activities for angiogenic disorders should be employed whenever possible.

When a crude extract is found to modulate CD39 biological activity, i.e., increase or decrease angiogenesis, further fractionation of the positive lead extract is necessary to isolate chemical constituents responsible for the observed effect. Thus, the goal of the extraction, fractionation, and purification process is the careful characterization and identification of a chemical entity within the crude extract having the CD39-modulating activity, for example, angiogenesis-reducing activities. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using a mammalian angiogenesis model or any other model described herein.

Primary Screens for Compounds that Modulate Angiogenesis by Modulating CD39 Biological Activity

We have discovered that activation of P2 receptors by CD39-mediated phosphohydrolysis of nucleoside di- and triphosphates appears to influence endothelial cell chemotactic and mitogenic responses in vitro. Therefore, aberrant regulation of nucleotide P2-receptors may influence new vessel growth. CD39 regulates P2 receptor activation by regulating the presence of nucleoside di- and triphosphates generated by CD39-mediated phosphohydrolysis. These findings allow us to provide assays for compounds that affect angiogenesis by modulating the activity or expression of CD39, or a signaling pathway associated with CD39 activity. Such assays measure various aspects of CD39 expression or activity, or signaling components associated with the P2 receptor. Such changes include: (a) expression of CD39; (b) phosphohydrolysis of nucleoside di- and triphosphates; (c) transmigration of monocyte-macrophages or endothelial cells; (d) activation status of the P2 receptor; (e) mRNA or polypeptide levels of components of the P2 receptor signaling pathway. These measurements form the basis of assays that can be used to identify compounds that modulate CD39-mediated angiogenesis. Such identified compounds may have therapeutic value, for example, in the treatment of angiogenesis-associated conditions or diseases.

One method in which test compounds that modulate CD39 biological activity, for example CD39-mediated angiogenesis, may be identified is to expose a cell expressing CD39 to a test compound, followed by assaying CD39 biological activity or gene expression. A decrease in CD39 biological activity or gene expression in the cell tested, relative to CD39 biological activity, or gene expression in a cell not exposed to the test compound, indicates a test compound that is capable of reducing CD39 biological activity, for example CD39-mediated angiogenesis. This methodology may be adjusted to identify test compounds by measuring one or more of the various aspects discussed above.

Expression of a reporter gene that is operably linked to a CD39 promoter can also be used to identify such candidate compounds. A reporter gene may encode a reporter enzyme that has a detectable read-out, such as beta-lactamase, beta-galactosidase, or luciferase. Reporter enzymes can be detected using methods known in the art, such as the use of chromogenic or fluorogenic substrates for reporter enzymes as such substrates are known in the art. Such substrates are desirably membrane permeant. Chromogenic or fluorogenic readouts can be detected using, for example, optical methods such as absorbance or fluorescence. A reporter gene can be part of a reporter gene construct, such as a plasmid or viral vector, such as a retrovirus or adeno-associated virus. A reporter gene can also be extra-chromosomal or it can be integrated into the genome of a host cell. The expression of the reporter gene can be under the control of exogenous expression control sequences or expression control sequences within the genome of the host cell. Under the latter configuration, the reporter gene is desirably integrated into the genome of the host cell.

Screening methods according to the invention may be carried out in any cell, for example, a cell (such as a mammalian cell) into which a heterologous CD39 gene or a CD39 reporter gene has been introduced by stable transfection, or a cell in which expression of CD39 is endogenous. Alternatively, these screens may be carried out in cells in which the CD39 gene is defective, has increased activity or expression, or is non-functional. In these cells, compounds that decrease or compensate for increased CD39 activity can be identified, as compounds that either decrease the level of CD39 expression or activity, decrease inappropriate vascularization or infiltration of monocyte-macrphages, or allow angiogenesis to return to near or completely normal levels. Characteristics that may be assayed include, without limitation, phosphohydrolysis of nucleoside di- and triphosphate (as described herein), CD39 expression or activity, transmigration and/or activation of monocyte-macrophages or endothelial cells, immunohistochemical analysis of angiogenesis-associated markers, or P2 receptor activation and signaling. Desirable compounds would be those that decrease expression or activation of CD39, reduce the phosphohydrolysis of nucleoside di- and triphosphates, reduce transmigration of monocyte-macrophages or endothelial cells to sites of inappropriate vascularization, or reduce P2 receptor activation.

Diagnosis of an Increased Risk of Disease Associated with CD39 Biological Activity

The present discovery that CD39 is a gene involved in regulating angiogenesis by regulating nucleotide-sensitive P2 receptors on macrophages, monocytes, and endothelial cells, and whose absence correlates with loss of new vessel growth facilitates novel assays for diagnosing whether subjects have angiogenesis-associated conditions or diseases, or a propensity toward developing those conditions or diseases. For example, an increased level of CD39 biological activity indicates that a subject has an increased risk of developing cancer or metastasis of cancer, a chronic inflammatory illness (for example, rheumatoid arthritis), inflammatory bowel disease, diabetic retinopathy, or chronic radiation-induced proctitis. A decreased level of CD39 biological activity indicates that a subject has an increased risk of developing peripheral vascular disease (e.g., atherosclerosis, thromboembolic disease, or Brueger's disease (thromboangiitis obliterans)), cardiovascular disease (e.g., myocardial infarction, heart disease, or coronary artery disease), tissue organ engraftment rejection, or sequelae of ischemic reperfusion injury. Diagnosis in this manner can be applied to a variety of conditions associated with CD39 biological activity in general. Those skilled in the art will appreciate that many different types of diagnostic assays are available for detection of a condition associated with a modulation in CD39 biological activity, for example, an angiogenic condition. For example, one or more mutations in the DNA of a patient that promote overexpression of CD39 or modulate the function of CD39 may correlate with disregulation of components of the vasculature and can be used to diagnose disease, for example the likelihood of inappropriate formation of new vessels in cancers. One skilled in the art can identify the formation of new vessels in a patient, acquire a sample from the patient and, using standard techniques, can determine the presence of a mutation in the CD39 gene. This information may be used to screen a population as a whole for individuals that are at an increased risk of developing a particular type of CD39-associated condition, for example, an angiogenic disorder, or may be used to test individual patients, for example, those with a family history of a CD39-associated condition, for example, an angiogenesis-associated condition, such as those listed above.

As described herein, CD39 has been shown to function as a regulator of angiogenesis. These results come from our study of angiogenesis in cd39-null mice. In view of our results, diagnosis of an angiogenesis-associated condition in a patient using CD39 as either a gene or polypeptide marker can be performed by assaying for a mutation in the expression or activity of the CD39 polypeptide (see below), or by determining the presence of a mutation in the CD39 gene (for example, in a tumor cell). A mutation can be determined by detecting the presence of a mutation in the gene sequence, or possibly the complete absence of the CD39 gene, gene expression, or polypeptide activity in a cell. This type of information may even be used to further characterize the likelihood of disease or to determine the prognosis of a patient who has been diagnosed with an angiogenesis-associated condition (see below).

As noted above, a mutation in the CD39 gene may be associated with, for example, an angiogenesis-associated condition such as cancer or metastasis of cancer, chronic inflammation (for example, rheumatoid arthritis), inflammatory bowel disease, diabetic retinopathy, chronic radiation-induced proctitis, peripheral vascular disease (e.g., atherosclerosis, thromboembolic disease, or Brueger's disease (thromboangiitis obliterans)), cardiovascular disease (e.g., myocardial infarction, heart disease, or coronary artery disease), tissue organ engraftment rejection, or sequelae of ischemic reperfusion injury. Specifically, a mutation in CD39 can be identified from blood or tissue samples from patients suspected to have a CD39-associated condition. Probes and primers based on the CD39 gene sequence or based on known mutations in the CD39 gene sequence can be used as markers to detect any mutation or loss of the CD39 gene in samples from patients. Probes or primers may be based on the human CD39 nucleic acid and amino acid sequence known in the art (see, U.S. Pat. No. 6,287,837; Maliszenski et al. (1994); Christoforidis et al. (1995)).

A mutation in the CD39 gene may be identified in a biological sample obtained from a patient using a variety of methods available to those skilled in the art. Generally, these techniques involve, for example, PCR amplification of nucleic acid from the patient sample, followed by identification of the mutation by either altered hybridization, aberrant electrophoretic gel migration, restriction fragment length polymorphism (RFLP) analysis, binding or cleavage mediated by mismatch binding proteins, or direct nucleic acid sequencing. Any of these techniques may be used to facilitate detection of a mutation in the CD39 gene, and each is well known in the art; examples of particular techniques are described, without limitation, in Orita et al. (Proc. Natl. Acad. Sci. USA 86:2766-2770, 1989) and Sheffield et al. (Proc. Natl. Acad. Sci. USA 86:232-236 (1989)).

Alternatively, a mutation in the CD39 gene may be assayed by detecting changes in CD39 expression, either at the RNA or protein levels. For example, expression of the CD39 gene in a biological sample may be monitored by standard Northern blot analysis (to examine mRNA levels) or may be aided by PCR (see, e.g., Ausubel et al., Current Protocols in Molecular Biology, John Wiley & Sons, New York, N.Y. (1994); PCR Technology: Principles and Applications for DNA Amplification, H.A. Ehrlich, Ed., Stockton Press, N.Y.; Yap et al., Nucl. Acids. Res. 19:4294 (1991)).

In addition, post-translational modifications, such as proteolysis, glycosylation (e.g., palmitoylation), and phosphorylation may also be responsible for modulating CD39 activity. Identifying these changes may also be important for diagnosing an angiogenesis-associated condition

In yet another alternative, antibodies directed against a CD39 protein may be used to detect altered expression levels of the protein, including a lack of expression, or a change in its mobility on a gel, indicating a change in structure or size. In addition, antibodies may be used for detecting an alteration in the expression pattern or the sub-cellular localization of the protein. The antibody may be used in immunoassays to detect or monitor protein expression, e.g., CD39 protein expression, in a biological sample. The antibody can be labeled, if desired, and used in standard immunoassays. A polyclonal or monoclonal antibody (produced as described below) may be used in any standard immunoassay format (e.g., ELISA, Western blot, or RIA) to measure polypeptide levels. These levels may be compared to normal levels. Examples of immunoassays are described, e.g., in Ausubel et al. (Current Protocols in Molecular Biology, John Wiley & Sons, New York, N.Y. (1994)).

Antibodies used in the present invention may include ones that recognize both the wild-type and mutant protein, as well as ones that are specific for either the wild-type or an altered form of the protein, for example, one encoded by a polymorphic or mutant CD39 gene. Monoclonal antibodies may be prepared using the CD39 protein described above and standard hybridoma technology (see, e.g., Kohler et al., Nature 256:495 (1975); Kohler et al., Eur. J. Immunol. 6:511 (1976); Kohler et al., Eur. J. Immunol. 6:292 (1976); Hammerling et al., In Monoclonal Antibodies and T Cell Hybridomas, Elsevier, New York, N.Y. (1981); Ausubel et al., Current Protocols in Molecular Biology, John Wiley & Sons, New York, N.Y. (1994)). Once produced, monoclonal antibodies are also tested for specific CD39 protein recognition by Western blot or immunoprecipitation analysis (by the methods described in, for example, Ausubel et al., Current Protocols in Molecular Biology, John Wiley & Sons, New York, N.Y. (1994)). Polyclonal antibodies that recognize wild type and mutant or polymorphic CD39 protein can also be generated, for example, in rabbits, goats, or mice using, for example, cDNA immunization, and used in the present invention for diagnosis of an angiogenesis-associated condition.

Antibodies used in the methods of the invention may be produced using amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson and Wolf (CABIOS 4:181 (1988)). These fragments can be generated by standard techniques, e.g., by PCR, and cloned into an expression vector, for example pGEX (Ausubel et al., Current Protocols in Molecular Biology, John Wiley & Sons, New York, N.Y. (1994)). GST fusion proteins can be made and expressed in E. coli and purified using a glutathione agarose affinity matrix as described in Ausubel et al. (Current Protocols in Molecular Biology, John Wiley & Sons, New York, N.Y., (1994)).

Antibodies or oligonucleotide probes/primers can be utilized in methods that are known to one skilled in the art to examine biological samples for the diagnosis of a CD39-associated condition, e.g., to diagnose cancer or inflammation, due to, for example, rheumatoid arthritis. Immunohistochemical techniques may also be utilized for protein detection. For example, a tissue sample may be obtained from a patient, sectioned, and stained for the presence of CD39 using an anti-CD39 antibody and any standard detection system (e.g., one which includes a secondary antibody conjugated to horseradish peroxidase). General guidance regarding such techniques can be found in, e.g., Bancroft and Stevens (Theory and Practice of Histological Techniques, Churchill Livingstone (1982); and Ausubel et al., Current Protocols in Molecular Biology, John Wiley & Sons, New York, N.Y. (1994)). This type of technique is particularly useful for detecting inappropriate cellular localization of the CD39 protein.

Diagnosis of an angiogenesis-associated condition can also be accomplished by determining the biological activity of the CD39 polypeptide. CD39 biological activity includes, for example, phosphohydrolysis of nucleoside di- and triphosphates which leads to-nucleotide-mediated signaling in the vasculature. This signaling can be verified by numerous methods known to those skilled in the art, including, for example, by assaying protein phosphorylation. Alternatively, biological activity may be measured by assaying for activation of monocyte-macrophage cells or endothelial cells, for example, by detecting monocyte-macrophage-specific secretory components, including TGF-b, TNF-a, interleukin-1 (IL-1), and metalloproteases. General guidance regarding these techniques can be found in standard laboratory manuals, such as Ausubel et al., Current Protocols in Molecular Biology, John Wiley & Sons, New York, N.Y., (1994), and Sambrook et al., Molecular Cloning: A Laboratory Manual, Cold Spring Harbor Laboratory, N.Y., (1989), and as described herein.

Immunohistochemical techniques can also be utilized for CD39 detection. For example, a tissue sample can be obtained from a patient, sectioned, and stained for the presence of CD39 using an anti-CD39 antibody and any standard detection system (i.e., one that includes a secondary antibody conjugated to horseradish peroxidase). General guidance regarding such techniques can be found in, e.g., Bancroft et al., Theory and Practice of Histological Techniques, Churchill Livingstone, 1982, and Ausubel et al., supra.

Prognosis of Patients Diagnosed with a CD39-Associated Condition

The present invention features a method for determining the prognosis for treatment of a patient diagnosed with a CD39-associated angiogenic condition, for example, cancer or metastasis of cancer, chronic inflammatory illneses (e.g., rheumatoid arthritis), inflammatory bowel disease, or chronic radiation-induced proctitis. The method utilizes the determination of the level of expression of CD39 mRNA or CD39 polypeptide, the phosphohydrolytic activity of CD39, or the detection of the presence of a mutation in a CD39 gene using a sample from the subject, for example, a biopsy. The methods presented in the preceding section, “Diagnosis of an Increased Risk of Disease Associated with CD39 Biological Activity,” can be utilized in the determination of a prognosis for treatment of a CD39-associated condition. Generally, a prognosis for a condition that is exacerbated by an increase in angiogenesis, for example cancer or metastasis of cancer, chronic inflammatory illnesses (e.g., rheumatoid arthritis), inflammatory bowel disease, diabetic retinopathy, or chronic radiation induced proctitis can be determined by detecting a change in the amount of a CD39 mRNA or polypeptide, as detected in, for example a tumor biopsy, by the methods discussed above. For these condition, an increase in CD39 biological activity (e.g., an increase in CD39 mRNA or polypeptide levels, or the phosphohydrolysis of nucleoside di- or triphosphates mediated by CD39), indicates a negative prognosis for the treatment of an angiogenesis-related condition, as this suggests the possibility that angiogenesis will occur in a more aggressive manner (for example, it may indicate that a tumor is more likely to metastasize to another location). Detection of decreased CD39 biological activity, by detecting mRNA or polypeptide levels, or protein activity, indicates a more positive prognosis, for example, for the conditions listed above.

Alternatively, a prognosis can also be made for a condition that would be improved by an increase in angiogenesis, for example peripheral vascular disease (e.g., atherosclerosis, thromboembolic disease, or Brueger's disease), cardiovascular disease (e.g., myocardial infarction, heart disease, or coronary artery disease), tissue organ engraftment rejection, or sequelae of ischemic reperfusion injury. Detection of an increase in CD39 biological activity, indicates a positive prognosis for the treatment of these angiogenesis-related conditions, while a decrease in CD39 biological activity indicates a negative prognosis.

Administration of CD39 or a Candidate Compound to Enhance Angiogenesis

The present invention includes methods for promoting or increasing angiogenesis, for example during transplantation and tissue organ engraftment, or for the treatment, stabilization, or prevention of, for example, peripheral vascular disease, cardiovascular disease, and sequelae of ischemic reperfusion injury. Desirably, CD39 or compounds that increase the activity of CD39 would be administered to boost CD39 biological activity (e.g., phosphohydrolysis of nucleoside di- and triphosphates).

Administration of an Inhibitor of CD39 or a Candidate Compound for the Treatment, Stabilization, or Prevention of an Angiogenesis-Associated Condition

The present invention includes methods for inhibiting or decreasing angiogenesis by administering a compound that inhibits or decreases CD39 biological activity or a compound that decreases CD39 expression. Desirably, an inhibitor of CD39 or a candidate compound that modulates a pathway related to CD39-mediated angiogenic activity would be administered to treat, stabilize, or prevent, for example, cancer or metastasis of cancer, chronic inflammation (associated with, for example, rheumatoid arthritis), diabetic retinopathy, inflammatory bowel disease, or chronic radiation-induced proctitis.

Gene Therapy

One example of a method for the treatment, stabilization, or prevention of an angiogenesis-associated condition can be gene therapy (see, generally, Morgan et al., Ann. Rev. Biochem. 62:191-217 (1993), Culver et al., Trends Genet.10:174-178 (1994), and U.S. Pat. No. 5,399,346 (French et al.)). The general principle is to identify one or more polypeptides which can be used to treat, stabilize, or prevent an angiogenesis-associated condition and to introduce the polynucleotide(s), for example a CD39 gene, into, for example, a cancer cell in a patient. Desirably, expression of the gene will decrease angiogenesis.

Transducing retroviral, adenoviral, and adeno-associated viral vectors can be used for somatic cell gene therapy, especially because of their high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641-6649 (1997); Naldini et al., Science 272:263-267 (1996); and Miyoshi et al., Proc. Natl. Acad. Sci., USA 94:10319-10332 (1997)). For example, the full length CD39 gene, or a portion thereof, can be cloned into a retroviral vector and expression can be driven from its endogenous promoter, from the retroviral long terminal repeat, or from a promoter specific for a target cell type of interest. Other viral vectors that can be used include, for example, vaccinia virus, bovine papilloma virus, or a herpes virus, such as Epstein-Barr virus (also see, for example, the vectors of Miller, Human Gene Therapy 1:5-14 (1990); Friedman, Science 244:1275-1281 (1989); Eglitis et al., Biotechniques 6:608-61₄ (1988); Tolstoshev et al., Current Opinion in Biotechnology 1:55-61 (1990); Sharp, The Lancet 337:1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995). Retroviral vectors are particularly well developed and have been used in clinical settings (Rosenberg et al., N. Engl. J. Med. 323:370 (1990); and Anderson et al., U.S. Patent No. 5,399,346.

Gene transfer can also be achieved using non-viral means involving transfection in vitro. Such methods include use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal CD39 gene into a cultivatable cell type ex vivo, after which the cell (or its descendants) are injected into a targeted tissue.

Non-viral approaches can also be employed for the introduction of therapeutic DNA into cells predicted to be subject to diseases involving CD39. For example, a CD39 nucleic acid molecule or an antisense nucleic acid molecule can be introduced into a cell by lipofection (Feigner et al., Proc. Natl. Acad. Sci. USA 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or, less preferably, micro-injection under surgical conditions (Wolff et al., Science 247:1465, 1990).

CD39 cDNA expression for use in gene therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct CD39 expression. The enhancers used can include, without limitation, those that are characterized as tissue- or cell-specific enhancers. Alternatively, if a CD39 genomic clone is used as a therapeutic construct (such clones can be identified by hybridization with CD39 cDNA, described above), regulation can be mediated by the cognate regulatory sequences, or, if desired, by regulatory sequences derived from a heterologous source, including any of the promoters or regulatory elements described above.

Entry into the cell is facilitated by suitable techniques known in the art as providing the polynucleotide in the form of a suitable vector, or encapsulation of the polynucleotide in a liposome. The polynucleotide may be provided to the site of angiogenesis by an antigen-specific homing mechanism, or by direct injection.

A desired mode of gene therapy is to provide the polynucleotide in such a way that it will replicate inside the cell, enhancing and prolonging the interference effect. Thus, the polynucleotide is operably linked to a suitable promoter, such as the natural promoter of the corresponding gene, a heterologous promoter that is intrinsically active in cancer cells, or a heterologous promoter that can be induced by a suitable agent.

In another aspect, the construct is designed so that the polynucleotide sequence operably linked to the promoter is complementary to the sequence of a corresponding gene. Thus, once integrated into the cellular genome, the transcript of the administered polynucleotide will be complementary to the transcript of the gene, and capable of hybridizing with it. This approach is known as anti-sense therapy. See, for example, Culver et al., Trends Genet. 10:174-178 (1994), and Roth, Ann. Surg. Oncol. 1:79-86 (1994). A retroviral vector can be used for therapeutic or diagnostic purposes in order to introduce into the patient a nucleotide sequence of clinical importance.

Peptide Agents

Peptide agents of the invention, such as a CD39 polypeptide, or other peptide compounds identified by the invention, can be administered to a subject, e.g., a human, directly or in combination with any pharmaceutically acceptable carrier or salt known in the art for the treatment, stabilization, or prevention of a CD39-associated condition. Pharmaceutically acceptable salts may include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. One exemplary pharmaceutically acceptable carrier is physiological saline. Other physiologically acceptable carriers and their formulations are known to one skilled in the art and described, for example, in Remington's Pharmaceutical Sciences, (18th edition), ed. A. Gennaro, 1990, Mack Publishing Company, Easton, Pa.

Pharmaceutical formulations of a therapeutically effective amount of a peptide agent or candidate compound of the invention, or pharmaceutically acceptable salt-thereof, can be administered orally, parenterally (e.g., intramuscular, intraperitoneal, intravenous or subcutaneous injection, inhalation, intradermally, optical drops, or implant), nasally, vaginally, rectally, sublingually or topically, in admixture with a pharmaceutically acceptable carrier adapted for the route of administration.

The polypeptide or candidate compound of the present invention can be prepared in any suitable manner. The polypeptide or candidate compound can be isolated from naturally occurring sources, recombinantly produced, or produced synthetically, or produced by a combination of these methods. The synthesis of short peptides is well known in the art. See, e.g., Stewart et al., Solid Phase Peptide Synthesis (Pierce Chemical Co., 2d ed., 1984).

Anti-CD39 Antibodies

Antibodies to CD39 proteins can be used, as noted above, to detect CD39 proteins or to inhibit the biological activities of CD39 proteins. For example, a nucleic acid molecule encoding an antibody or portion of an antibody can be expressed within a cell to inhibit CD39 function. In addition, the antibodies can be coupled to compounds, such as radionuclides and liposomes for diagnostic or therapeutic uses. Antibodies that specifically recognize extracellular domains of CD39 are useful for targeting such attached moieties to cells displaying such CD39 polypeptide domains at their surfaces. Antibodies that inhibit the activity of a CD39 polypeptide described herein can also be useful in preventing or slowing the development of a disease caused by inappropriate expression of a wild type or mutant CD39 gene.

Antisense Oligonucleotides

The specificity and sensitivity of antisense is also harnessed by those of skill in the art for therapeutic uses. Antisense oligonucleotides have been employed as drugs in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans. Proposed mechanisms of action include inhibition of the following: RNA synthesis, RNA splicing, mRNA export, binding of initiation factors or assembly of ribosome subunits and their migration.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, continuous infusion, subcutaneous, intraperitoneal or intramuscular injection, pulmonary administration, e.g., by inhalation or insufflation, or intrathecal or intraventricular administration. For oral administration, it has been found that oligonucleotides with at least one 2′-substituted ribonucleotide are particularly useful because of their absorption and distribution characteristics. Oligonucleotides with at least one 2′-methoxyethyl modification are believed to be particularly useful for oral administration.

Methods well known in the art for making formulations are found, for example, in Remington's Pharmaceutical Sciences (18th edition), ed. A. Gennaro, 1990, Mack Publishing Company, Easton, Pa. Compositions intended for oral use may be prepared in solid or liquid forms according to any method known to the art for the manufacture of pharmaceutical compositions. The compositions may optionally contain sweetening, flavoring, coloring, perfuming, and/or preserving agents in order to provide a more palatable preparation. Solid dosage forms for oral administration include capsules, tablets, pills, powders, and granules. In such solid forms, the active compound is admixed with at least one inert pharmaceutically acceptable carrier or excipient. These may include, for example, inert diluents, such as calcium carbonate, sodium carbonate, lactose, sucrose, starch, calcium phosphate, sodium phosphate, or kaolin. Binding agents, buffering agents, and/or lubricating agents (e.g., magnesium stearate) may also be used. Tablets and pills can additionally be prepared with enteric coatings.

Liquid dosage forms for oral administration include pharmaceutically acceptable emulsions, solutions, suspensions, syrups, and soft gelatin capsules. These forms contain inert diluents commonly used in the art, such as water or an oil medium. Besides such inert diluents, compositions can also include adjuvants, such as wetting agents, emulsifying agents, and suspending agents.

Formulations for parenteral administration include sterile aqueous or non-aqueous solutions, suspensions, or emulsions. Examples of suitable vehicles include propylene glycol, polyethylene glycol, vegetable oils, gelatin, hydrogenated naphalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for the polypeptides of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the compositions. Alternatively, they can also be manufactured in the form of sterile, solid compositions which can be dissolved in sterile water or some other sterile injectable medium immediately before use.

Compositions for rectal or vaginal administration are desirably suppositories which may contain, in addition to active substances, excipients such as coca butter or a suppository wax. Compositions for nasal or sublingual administration are also prepared with standard excipients known in the art. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops or spray, or as a gel.

The amount of active ingredient in the compositions of the invention can be varied. One skilled in the art will appreciate that the exact individual dosages may be adjusted somewhat depending upon a variety of factors, including the compound being administered, the time of administration, the route of administration, the nature of the formulation, the rate of excretion, the nature of the subject's conditions, and the age, weight, health, and gender of the patient. Generally, dosage levels of between 0.1 μg/kg to 100 mg/kg of body weight are administered daily as a single dose or divided into multiple doses. Desirably, the general dosage range is between 250 μg/kg to 5.0 mg/kg of body weight per day. Wide variations in the needed dosage are to be expected in view of the differing efficiencies of the various routes of administration. For instance, oral administration generally would be expected to require higher dosage levels than administration by intravenous injection. Variations in these dosage levels can be adjusted using standard empirical routines for optimization, which are well known in the art. In general, the precise therapeutically effective dosage will be determined by the attending physician in consideration of the above identified factors.

The candidate compound of the invention can be administered in a sustained release composition, such as those described in, for example, U.S. Pat. No. 5,672,659 and U.S. Pat. No. 5,595,760. The use of immediate or sustained release compositions depends on the type of condition being treated. If the condition consists of an acute or over-acute disorder, a treatment with an immediate release form will be desired over a prolonged release composition. Alternatively, for preventative or long-term treatments, a sustained released composition will generally be desired.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

The following examples are meant to illustrate the invention. They are not meant to limit the invention in any way.

### EXAMPLE 1

CD39 Regulates Angiogenesis

Angiogenesis Model

Four MATRIGEL® plugs containing growth factors/additives from cd39-null mice (described in Enjyoji et al. (1999), and incorporated fully by reference) and 4 matched MATRIGEL® samples from wild-type mice were harvested at day 7. Six MATRIGEL® plugs containing all 3 additives from cd39-null mice and 6 MATRIGEL® plugs from wild-type mice were harvested at day 14. Four MATRIGEL® plugs containing all 3 additives from cd39-null mice and 4 MATRIGEL® plugs from wild-type mice were harvested at day 21. Two wild-type or mutant mice for each time point were injected with MATRIGEL® plugs containing only vascular endothelial growth factor (VEGF)/sphingosine-1-phosphate (SPP) and were also analyzed in parallel.

Tissues were analyzed at the 14-day point with regard to differences in alterations in vascular density at the interface between MATRIGEL® and underlying tissue, as well as observed ingrowth of vessels into the matrix (n=6).

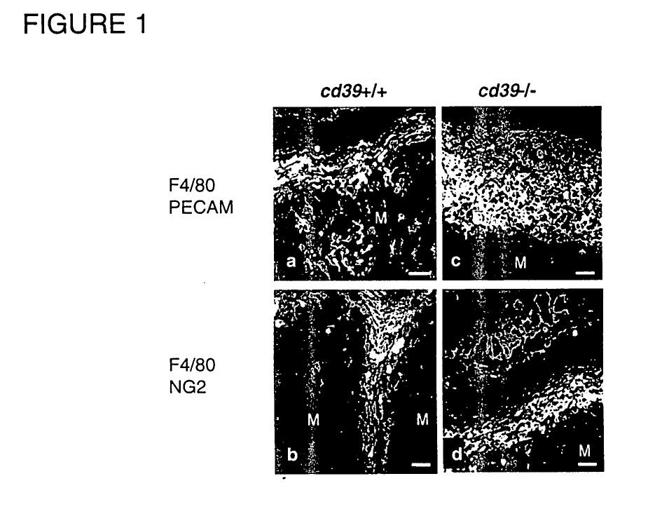

Wild-type mice displayed an increased vessel density at the interface as well as demonstrating substantial ingrowth of vessels into the MATRIGEL® itself (FIGS. 1A and 1C). Native vessels in the adjacent normal skin displayed signs of activation, indicated by the development of thin-walled, pericyte-poor vessels, called “mother vessels,” which stained for CD31 (FIGS. 1A and 1C). Newly formed vessels to a large extent were surrounded by NG2 and PDGF-receptor-expressing pericytes and were invested in a basement membrane, indicated by the presence of both perlecan and laminin (not shown). NG2 and PDGF-receptor-expressing pericytes, as well as monocyte/macrophages, were present at the leading edges of these connective-tissue septa. This pattern was followed by ingrowth of CD31-expressing endothelium (FIGS. 1A and 1C, Table I).

In contrast, cd39-null mice did not develop fill angiogenic responses at the interface or within the MATRIGEL® itself (FIGS. 1B and 1D). Vessels adjacent to the smooth muscle layer of the normal tissue showed some initial angiogenic responses, as indicated by the development of mother-vessel formation. Decreases in the expression of basement membrane components as well as increases in expression of the activation markers, i.e., KDR, PDGF-receptors, NG2, and αᵥβ₃ were noted in the cd39-null mice, albeit to a lesser degree than observed in the wild-type mice (Table I). Migration of endothelium toward the interface and into the MATRIGEL® was absent in the cd39-null mice (FIGS. 1B and 1D).

TABLE I
Distribution of Cell-Type-Specific Markers and Secreted Basal Lamina
Components in Wild-Type and cd39-Null Mice in the Matrigel Angiogenesis Assay

| | Normal | Mother | Vascular | Inflammatory | Matrigel | |
|---|---|---|---|---|---|---|
| | Vessels | Vessels | Zone | Zone | Septa | Matrigel |
| Endothelium | | | | | | |
| PECAM-1 | | | | | | |
| Wild-type | + | + | + | + | + | + |
| Null | + | + | −/+ | −/+ | 0 | 0 |
| VE-cadherin | | | | | | |
| Wild-type | + | −/+ | −/+ | −/+ | −/+ | + |
| Null | + | −/+ | −/+ | 0 | 0 | 0 |
| VEGF receptor 2 | | | | | | |
| Wild-type | + | + | + | + | + | + |
| Null | + | + | + | 0 | 0 | 0 |
| Monocyte/macrophages | | | | | | |
| F4/80 | | | | | | |
| Wild-type | NA | NA | + | + | + | + |
| Null | NA | NA | −/+ | + | 0 | 0 |
| Pericytes/myofibroblasts | | | | | | |
| α-Smooth muscle actin | | | | | | |
| Wild-type | −/+ | + | + | + | + | + |
| Null | −/+ | + | −/+ | 0 | 0 | 0 |
| PDGF-β receptors | | | | | | |
| Wild-type | −/+ | + | + | + | + | + |
| Null | −/+ | + | −/+ | 0 | 0 | 0 |
| NG2 | | | | | | |
| Wild-type | −/+ | + | + | + | + | + |
| Null | −/+ | + | −/+ | 0 | 0 | 0 |
| Basal lamina | | | | | | |
| Laminin | | | | | | |
| Wild-type | + | −/+ | −/+ | −/+ | + | + |
| Null | + | −/+ | −/+ | 0 | 0 | 0 |
| Perlecan | | | | | | |
| Wild-type | + | −/+ | −/+ | −/+ | + | + |
| Null | + | −/+ | −/+ | 0 | 0 | 0 |

0 indicates no expression; −/+, sporadic expression; +, abundant expression; and NA, not applicable.

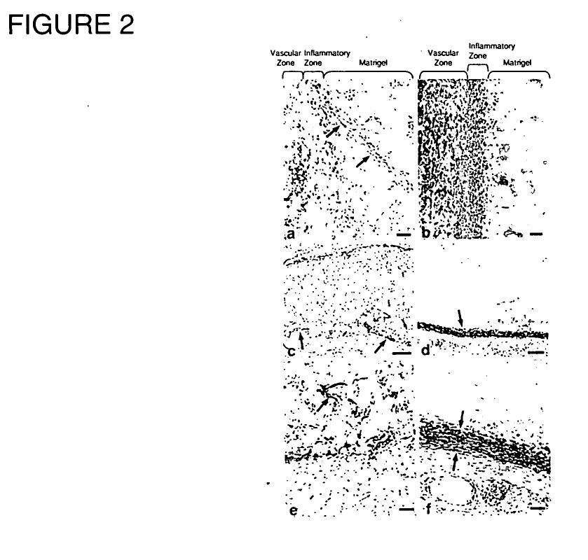

In addition, pericyte and macrophage infiltration was absent in the null mice (FIGS. 2B, 2D, and 2F). An interface between the dermis and the MATRIGEL® in wild-type mice was associated with connective-tissue septa penetrating into the MATRIGEL® and high cellularity within the MATRIGEL® itself (FIG. 2A); several of the unlabeled cells are considered fibroblasts. A compact cellular rim constituting the inflammatory zone at the interface between the dermis and the MATRIGEL®, as well as the lack of cells within the MATRIGEL® itself, was observed in cd39-null mice (FIGS. 2C and 2E). Marked F4/80-positive macrophage infiltration into the MATRIGEL® itself in wild-type mice contrasted with a compacted rim of F4/80-positive macrophages in the dermis at the dermis/MATRIGEL® interface in cd39-null mice (FIGS. 2C and 2E versus 2D and 2F).

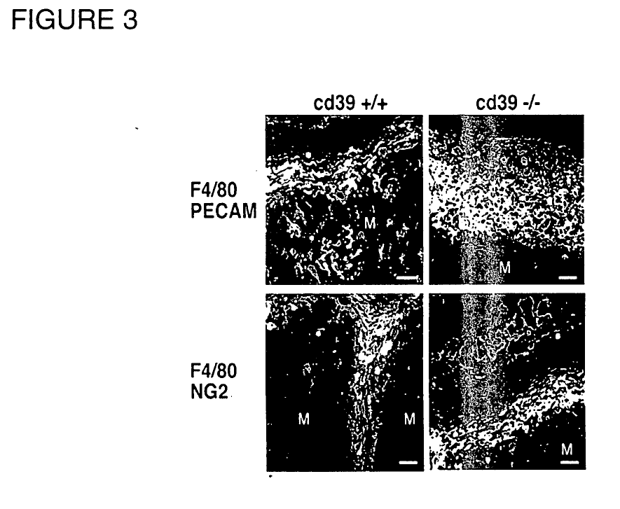

On closer examination, null monocytes, endothelial cells, and pericytes were present in distinct monocellular sandwich-type layers that approximated the interface between the normal tissue and the MATRIGEL® (FIGS. 3A-3D).

NTPDase Activity

The ATPase (ADPase) activity of the wild-type macrophages approximated 2090±200 (1780±110) Pᵢ nmol·min¹·well, whereas cd39-null macrophage activity was measured at 90±10 (30±10) Pᵢ nmol min¹·well (n=6; P<0.05).

Cytofluorometric Analyses

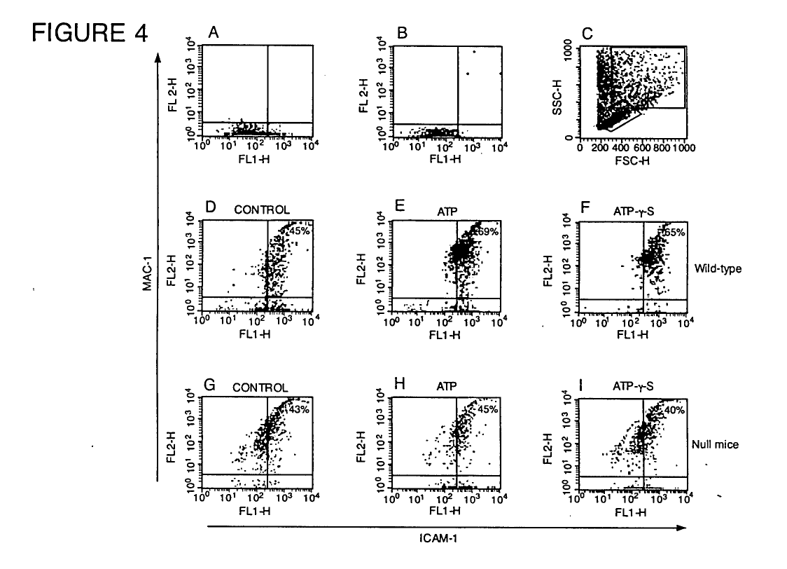

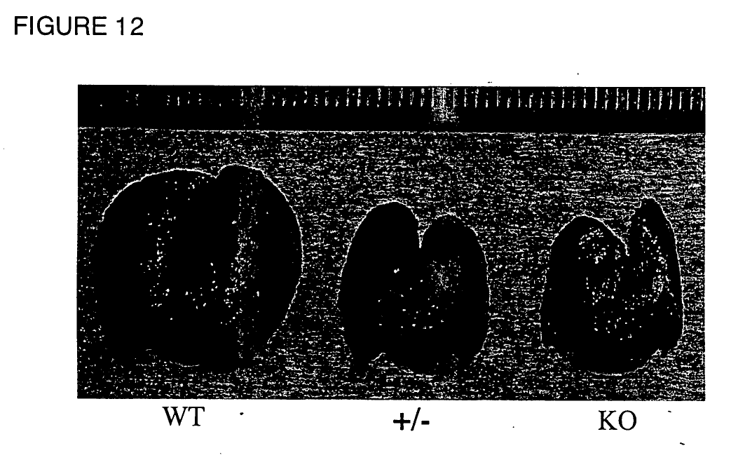

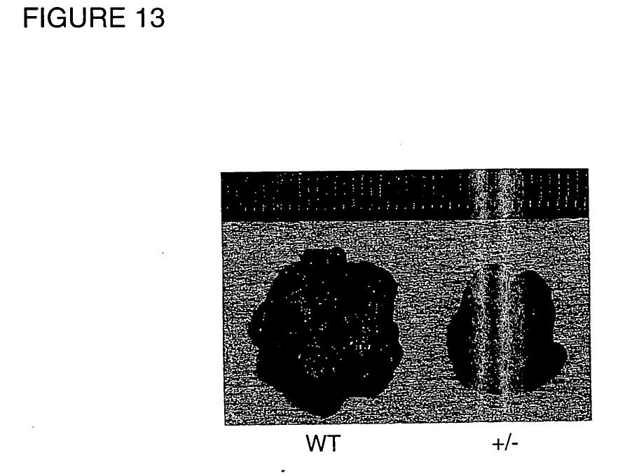

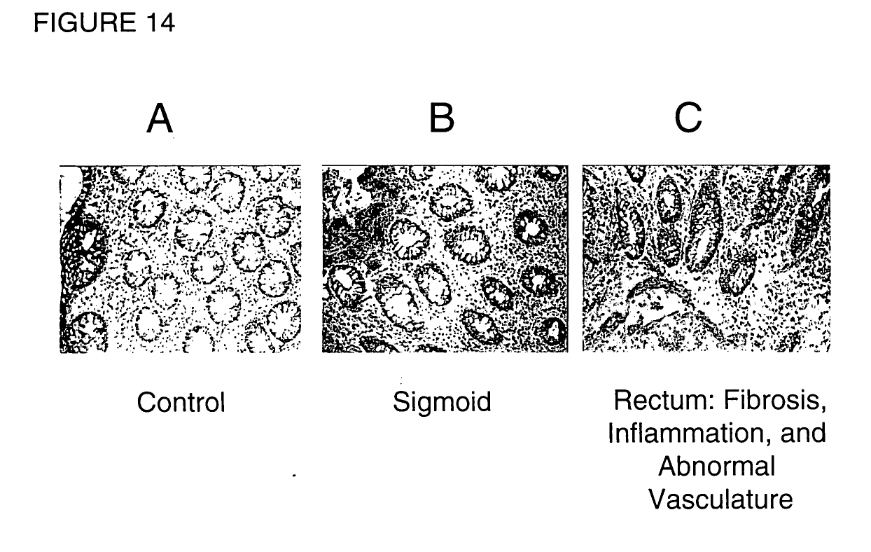

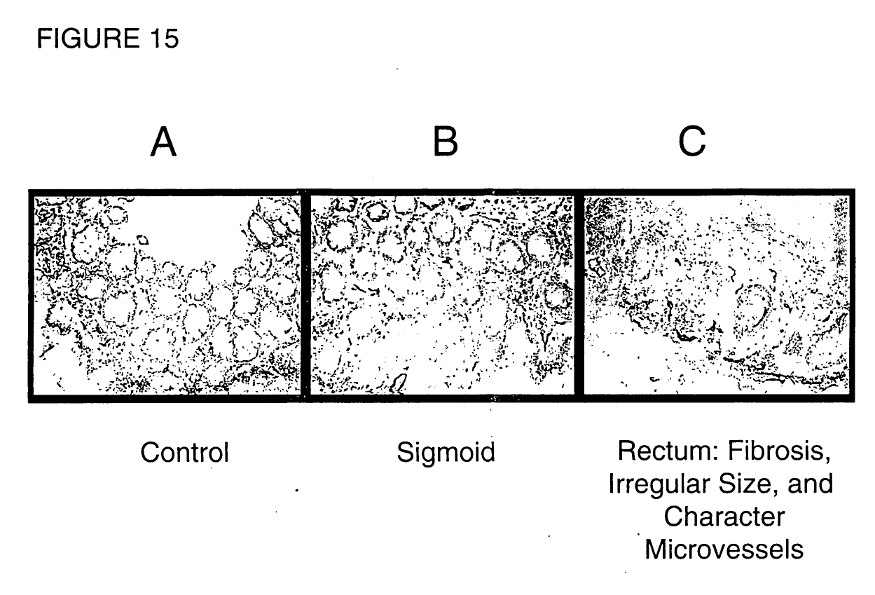

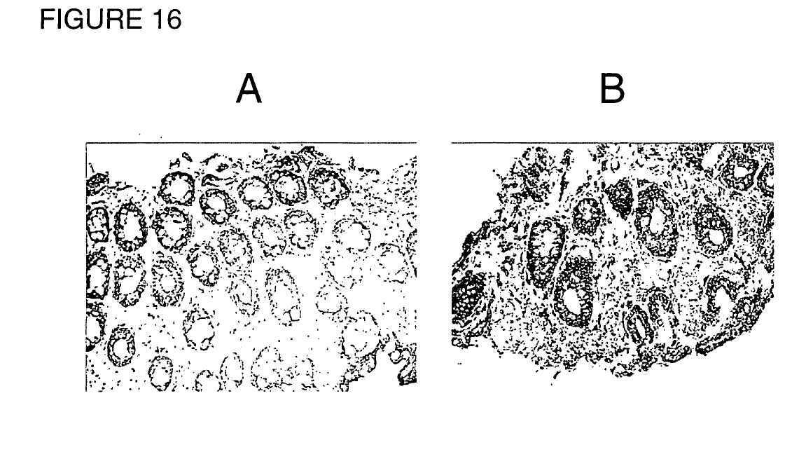

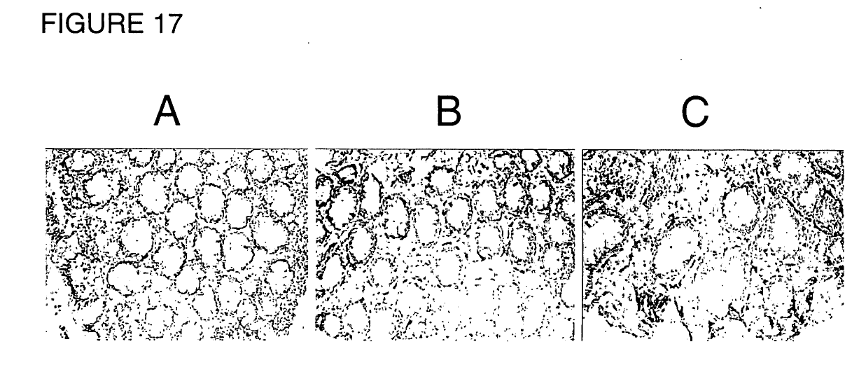

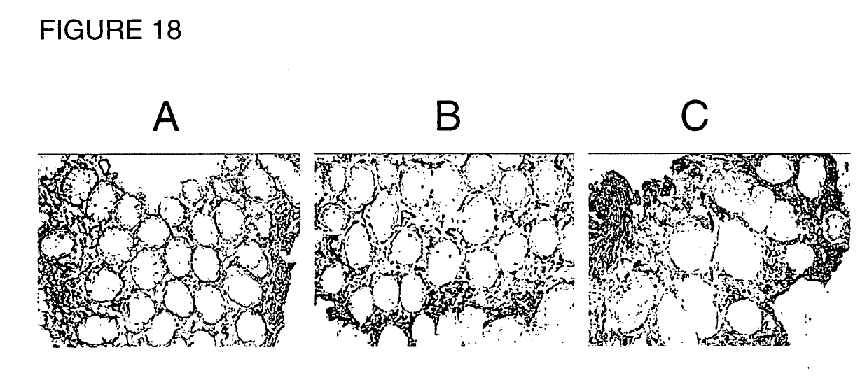

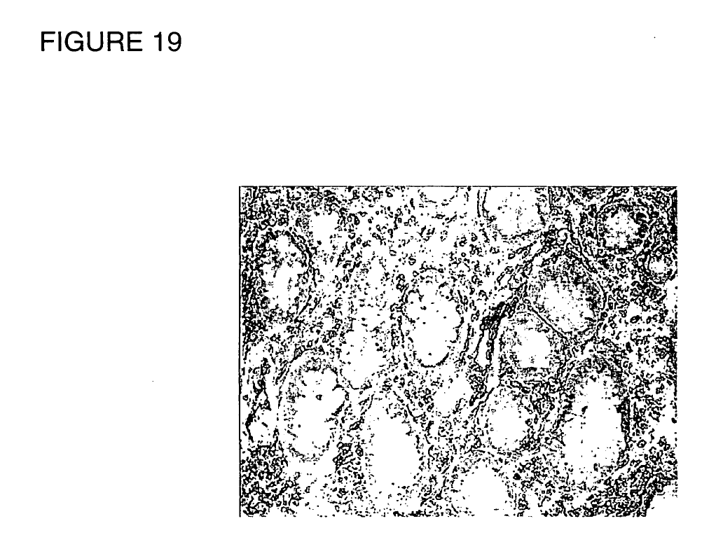

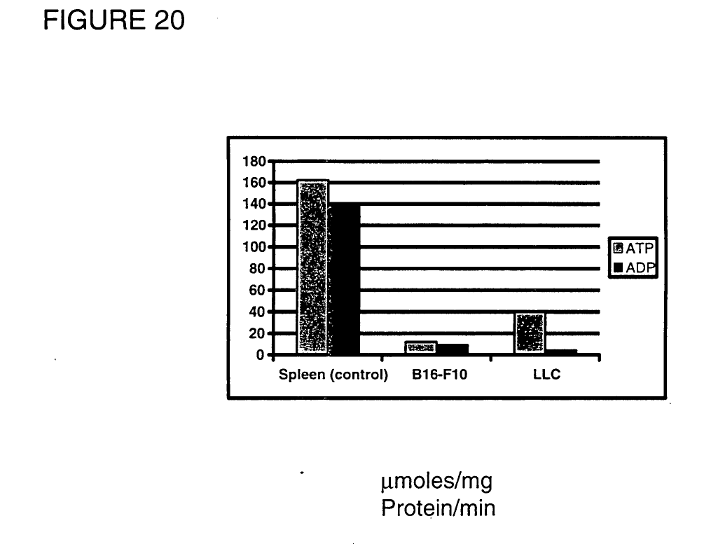

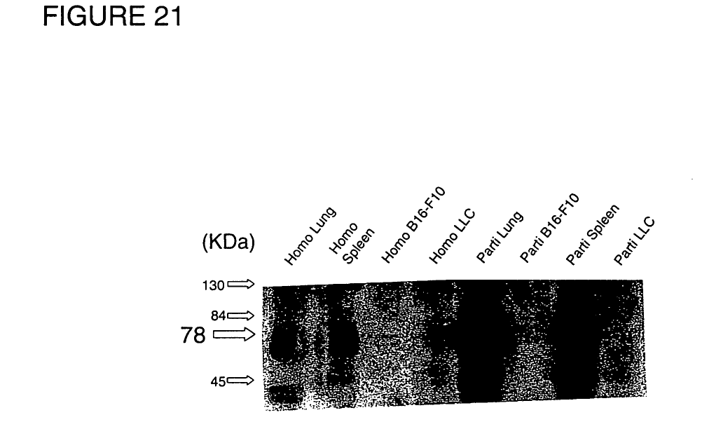

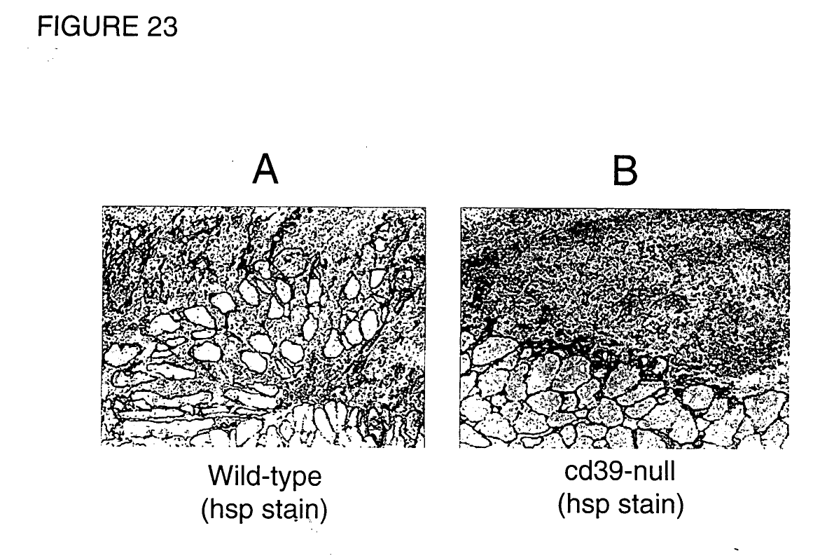

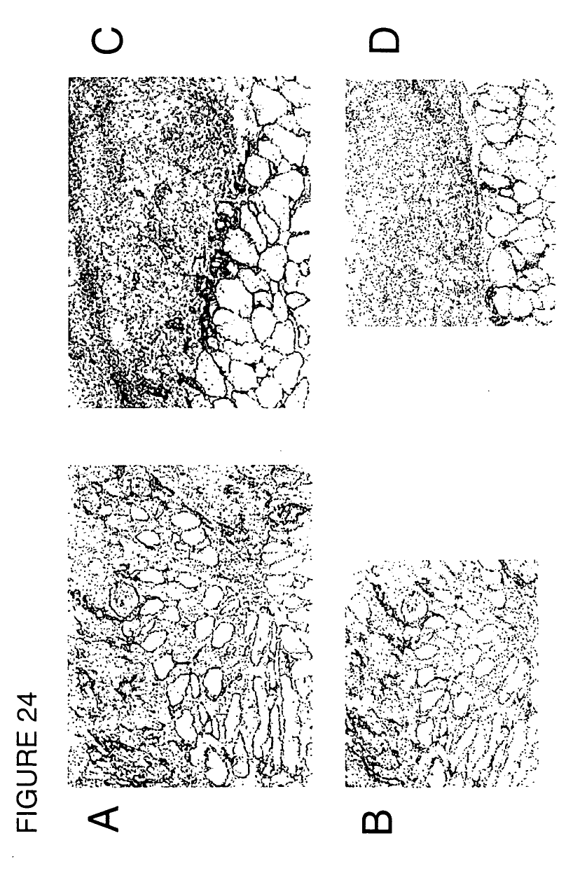

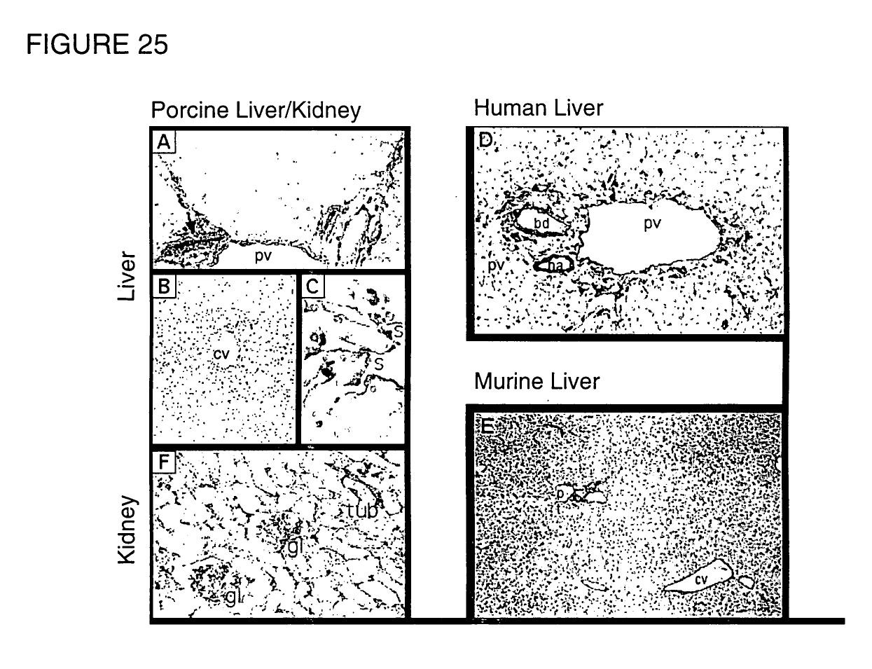

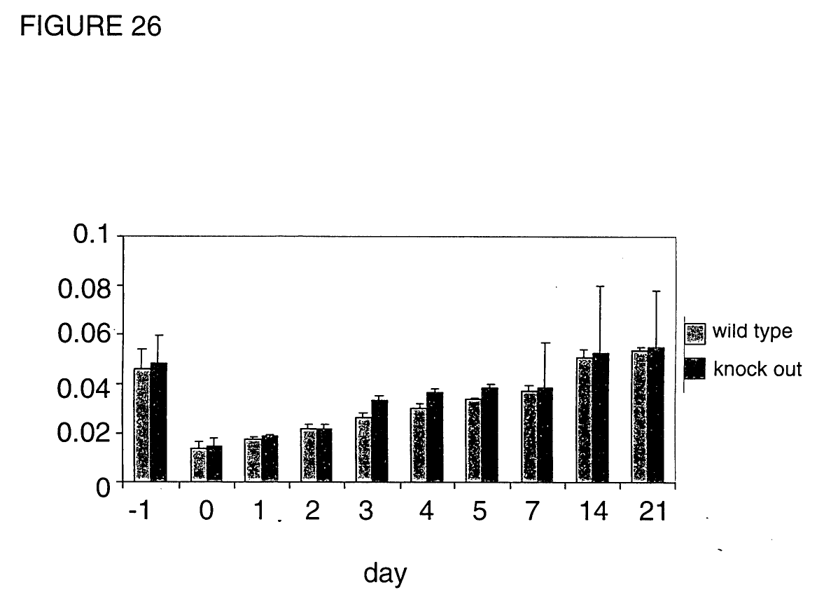

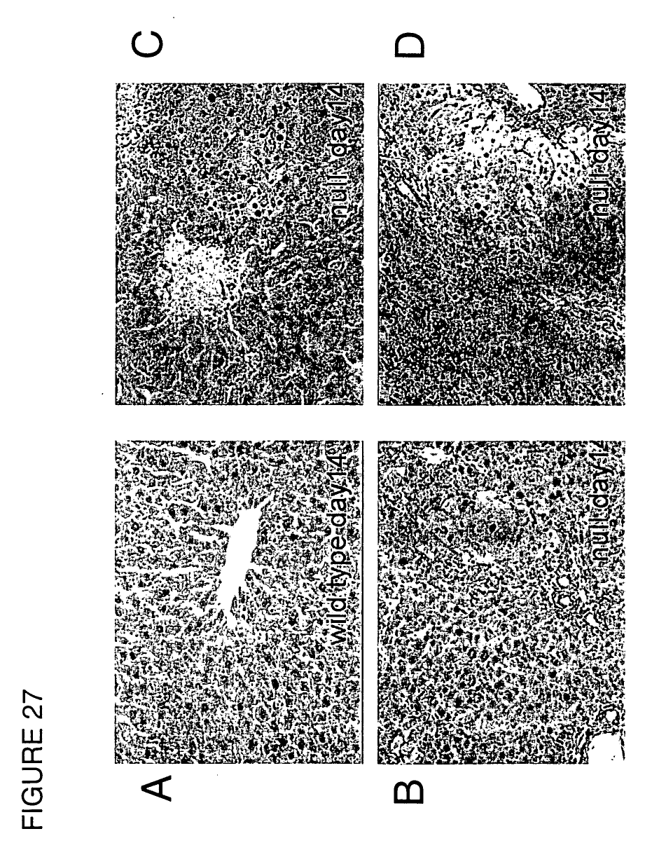

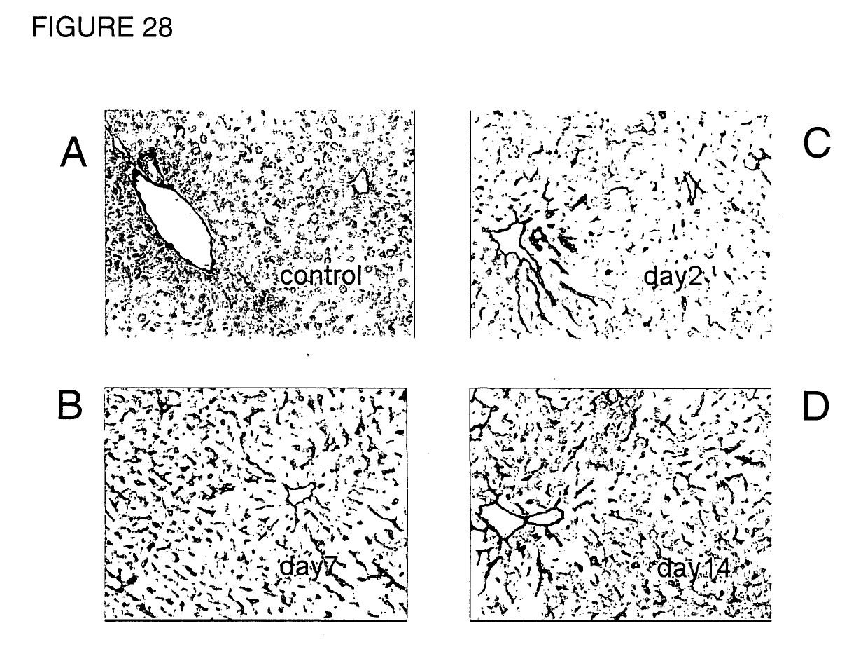

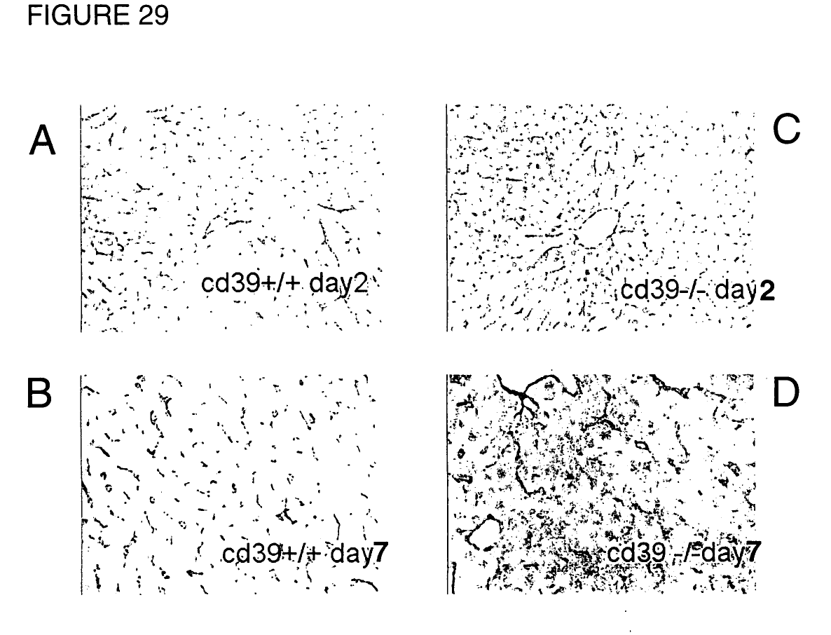

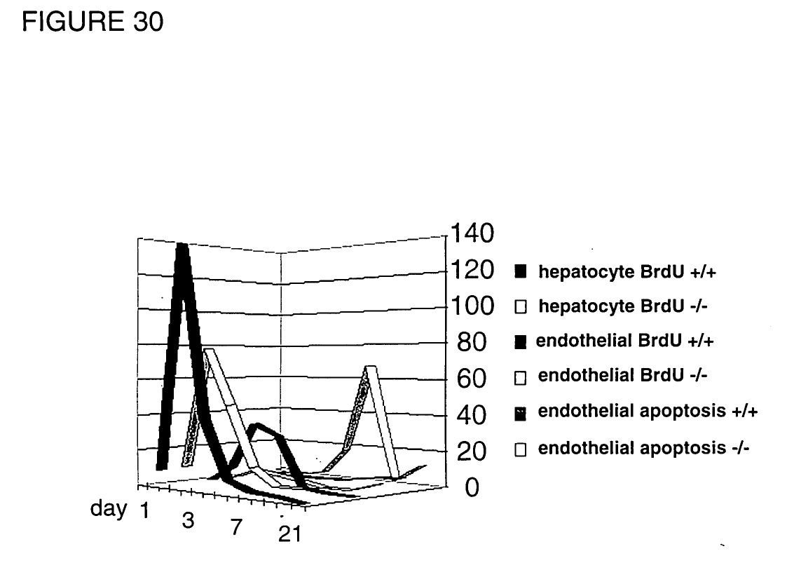

Median levels of wild-type macrophage cell surface coexpression of αₘβ₂ (MAC-1) and ICAM-1 increased significantly (n=6; P<0.05) after stimulation with 200 μmol/L ATP or ATPγS (30 minutes; n=6). In contrast, αₘβ₂ and ICAM-1 coexpression levels in quiescent cd39-null macrophages were largely unaltered after either ATP or ATPγS stimulation, respectively; an increase was not statistically significant compared with the basal levels (FIGS. 4A-41).

Transmigration of Monocyte/Macrophages Through Endothelial Cell Barriers, Collagen, and MATRIGEL® Matrices

Spontaneous migration of wild-type monocyte/macrophages across the endothelial cell line monolayer approximated 4900±400 cells. Control monocyte/macrophages also migrated toward extracellular ATP (200 μmol/L; 22,300±3,300). In response to MCP-1 (50 ng/mL), greater numbers of wild-type monocyte/macrophages migrated to the lower chamber across the monolayer (61,800±23,400; n=6). Mutant cd39-null monocyte/macrophages showed comparable levels of spontaneous migration at 6200±1200. cd39-null macrophages, however, migrated poorly toward a source of extracellular ATP (200 μmol/L; 8,750±4,100 P<0.05; n=4). The migratory response of null cells to MCP-1 (35,200±7,300) was less than wild-type cellular migration responses (n=6, P<0.05).

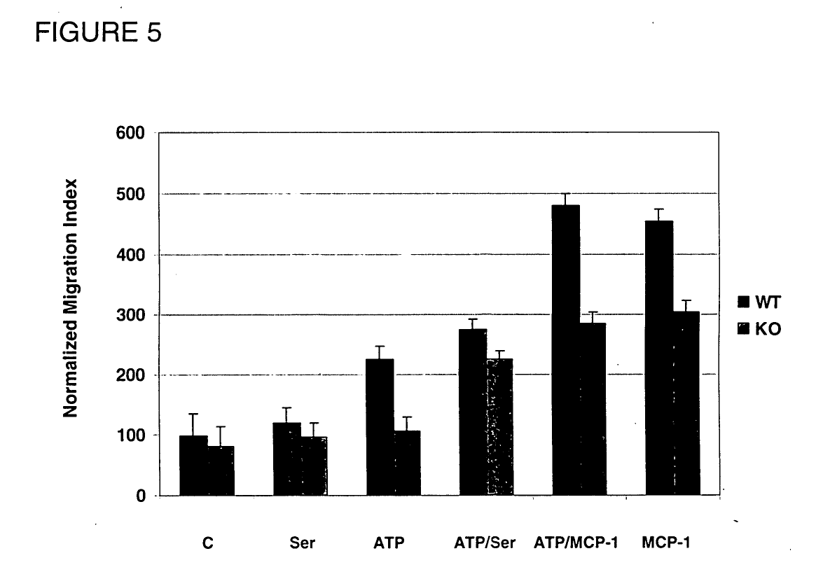

Transmigration of wild-type and mutant macrophages was then tested on MATRIGEL®-coated Transwell membranes (and collagen; not shown), in the absence of endothelial cells. As before, wild-type macrophages had low baseline migration levels; serotonin stimulation alone (20 μmol/L) did not substantially change the number of migrating macrophages (normalized to 120±20%), but ATP stimulation resulted in an increase of transmigrated cells (230±20%; P<0.05 and n=6 [FIG. 5]). Costimulation with ATP and serotonin substantially boosted migration of wild-type cells to levels of 280±20%. MCP-1 alone increased migration to 450±20%, whereas MCP-1 in combination with ATP effected levels of 480±20%; n=6 (FIG. 5). The number of nonstimulated cd39-null macrophages that spontaneously migrated was 80±30.0% of wild-type cells. Serotonin alone had no substantial effect on migration of cd39-null macrophages (98±20.0%).

In contrast to wild-type macrophages, ATP stimulation did not significantly increase the transmigration of cd39-null macrophages (108±24%; n=6, not significant). ATP in combination with serotonin, however, significantly boosted migration of cd39-null macrophages to 219±19% (P<0.05; n=6). MCP-1 alone and MCP-1 in combination with ATP showed lesser effects on the transmigration of cd39-null macrophages (303±17% and 282±22%, respectively) and relative to wild-type controls (FIG. 5).

The cd39-null mice have been shown to develop increased vascular permeability with tissue fibrin sequestration (Enjyoji, K., et al. Nat. Med. 5:1010-1017, 1999), which might have resulted in heightened angiogenic responses in vivo (Ciano, P. S., et al. Lab. Invest 54:62-70, 1986; Dvorak, H. F., et al. Lab. Invest. 57:673-686, 1987). However, we demonstrated almost total failure of the angiogenic response within the MATRIGEL® plugs containing SPP, FGF-2, and VEGF in vivo (FIGS. 1A-1D and FIGS. 2A-2F). Interestingly, there was complete stratification of the mutant cellular infiltrate at the MATRIGEL®-tissue interface and an ordered distribution into the monocyte/macrophage, endothelial cell, and pericyte layers. This pattern suggested a stereotyped cellular migration into the MATRIGEL® and emphasized the interdependence of these cells in the orchestration of the angiogenic responses in vivo (FIGS. 3A-3D)(Carmeliet, P. Nat. Med. 6:389-395, 2000). This observation is not unique to this model tested, because we have recently also shown disordered angiogenic responses to murine tumors when the cells were injected into the cd39-null mice (T. H., unpublished data).

Primary monocyte/macrophage abnormalities with dysfunctional recruitment, activation, and/or migration into the MATRIGEL® might provide an explanation for the lack of angiogenesis seen in cd39-null mice. The deletion of cd39 removed the major NTPDase activity at the cell surface of monocyte/macrophages. In keeping with published data (Akbar, G. K. M., et al. Biochem. Biophys. Res. Commun. 233:71-75, 1997), extracellular ATP upregulated αₘβ₂ expression on control monocyte/macrophages; in contrast, ATP failed to induce surface expression of αₘβ₂ on macrophages from cd39-null mice (FIGS. 4G-4I). We then demonstrated that ATP had potent chemoattractive potential for wild-type monocyte/macrophages in vitro. Importantly, the migratory potential of cd39-null macrophages in response to ATP (or MCP-1) was substantially decreased compared with control monocyte/macrophages (FIG. 5). Moreover, in contrast to the wild-type cells, ATP failed to attract cd39-null macrophages and promote migration through the MATRIGEL® in vitro (FIG. 5).

Abnormalities in monocyte/macrophage regulation of integrins and chemotactic responses (FIGS. 4A-4G and FIG. 5) suggested that G protein-coupled P2Y-receptor desensitization responses may have occurred after deletion of cd39, as previously observed for platelet P2Y1 (Enjyoji, K., et al. Nat. Med. 5:1010-1017, 1999). Therefore, we examined the effects of serotonin, in combination with ATP, to bypass certain P2Y-mediated pathways, as previously validated for platelet aggregation (Enjyoji, K., et al. Nat. Med. 5:1010-1017, 1999). Serotonin acts via unique G protein-coupled receptors on macrophages (Stephens, C. G., and R. Snyderman. J. Immunol. 128:1192-1197, 1982). Costimulation with serotonin and ATP rapidly restored the migratory responsiveness of cd39-null macrophages through the MATRIGEL® (FIG. 5). The exact mechanisms for this remain under evaluation, but P2Y-sequestration and phosphorylation reactions have been demonstrated to be associated with this phenomenon (Velazquez, B., et al. Mol. Cell. Biochem. 206:75-89, 2000).

A further explanation for the failure of angiogenesis would be a primary inability of microvessels to react to appropriate angiogenic stimuli. Against this possibility is the observation that initial early events in angiogenesis were demonstrated in mutant mice, e.g., mother-vessel formation (Paku, S. and K. Lapis. Am. J. Pathol. 143:926-936, 1993), degradation of the basal lamina, and decreased expression of VE-cadherin (Voura, E. B., et al. Microsc. Res. Tech. 43:265-275, 1998)(FIGS. 1A-1D and FIGS. 2A-2F; Table I). Endothelium in cd39-null mice expressed CD31 (FIGS. 1A-1D) and the αᵥβ₃ integrin (Table I) that mediates adhesion and migration with respect to several components of the provisional matrix (Varner, J. A., et al. Cell. Adhes. Commun. 3:367-374, 1995; Luscinskas, F. W., and J. Lawler. FASEB J. 8:929-938, 1994). Further analysis of functional activity of vascular endothelial integrins will be evaluated in the future.

Exogenous growth factors are implicated in the progressive neovascularization of MATRIGEL® plugs in vivo. The importance of FGF-2 in this phenomenon is emphasized by the observation that MATRIGEL® containing only VEGF and SPP failed to induce migration of blood vessels into the MATRIGEL® (C. S., unpublished data). FGF-2 is thought to exert its proangiogenic effects either directly on endothelium or through an indirect effect via activation of monocytes/supporting cells (Carmeliet, P. Nat. Med. 6:389-395, 2000; Lee, M. J., et al. Cell 99:301-312, 1999).

Our findings demonstrate that the phosphohydrolysis of extracellular nucleotides is important for both regulation of the cellular infiltrate at the initiation of angiogenesis and the progression of neovascularization. These observations may be pertinent to the understanding of new vessel growth in such human disease processes as cancer, rheumatoid arthritis, diabetic retinopathy, inflammatory bowel disease, and chronic radiation-induced proctitis.

Materials and Methods

Antibodies and Other Reagents

The polyclonal antibody anti-rat NG2 recognizes murine chondroitin sulfate proteoglycan, expressed on activated pericytes (Levine, J. M. and A. Nishiyama. Perspect. Dev. Neurobiol. 3:245-259, 1996); the macrophage marker used was rat F4/80 from Serotec. The monoclonal antibodies directed at mouse heparan sulfate proteoglycan (perlecan) and anti-laminin-β2 chain were purchased from Chemicon or Pharmingen. The fluorescein-labeled monoclonal antibody anti-smooth muscle α-actin (clone 1A4) was used as a marker for pericytes and smooth muscle cells (Skalli, O., et al. J. Submicrosc. Cytol. 18:481-493, 1986). Biotinylated monoclonal anti-body anti-mouse CD31, recognizing platelet and endothelial cell adhesion molecule (PECAM)-1, anti-β₃ integrin subunit, and anti-mouse CD144, recognizing VE cadherin, were purchased from Pharmingen. The polyclonal antibody rabbit anti-mouse platelet-derived growth factor (PDGF)-β receptor (Clone 958) was purchased from Santa Cruz Biotechnology. Polyclonal antibodies to vascular endothelial growth factor (VEGF) receptor-2 were from R. Brekken and P. Thorpe (University of Texas Southwestern Medical Center, Dallas). Biotinylated rabbit anti-mouse (Fab9)₂ and biotinylated pig anti-rabbit (Fab9)₂ were purchased from Dako.

Biotinylated rabbit anti-rat IgG, fluorescein-conjugated goat anti-rabbit IgG, rhodamine-conjugated rabbit anti-rat IgG, and Texas Red avidin D were from Vector Laboratories. Normal rabbit, mouse, swine, and goat serum and nonimmune rat, rabbit, and mouse IgG were purchased from Sigma. The terminal deoxynucleotidyl transferase apoptosis kit and the proliferating cell nuclear antigen staining kit were purchased from R&D systems and Zymed and used according to the manufacturers' instructions.

In Vivo Angiogenesis Assay

Mutant mice deficient in cd39 on the C57BL/6×129 svj strain were generated, validated, and characterized as we have described previously (Enjyoji, K., et al. Nat. Med. 5:1010-1017, 1999; Imai, M., et al. Mol. Med. 5:743-752, 1999); age- and sex-matched wild-type animals (C57BL/6×129 svj strain) were from Taconic. The animal experimentation protocol was reviewed and approved by the Animal Care and Use Committees of the Beth Israel Deaconess Medical Center. Mice were injected with 200 μL of MATRIGEL® (Costar, Fisher Scientific) at a final concentration of 9.9 mg/mL, containing 1.4 μg/mL VEGF, 8 μg/mL fibroblast growth factor (FGF)-2, 116 μg/mL BSA (fatty acid-free) purchased from Sigma, and 500 mmol/L SPP from Biomol (Lee, O. H., et al. Biochem. Biophys. Res. Commun. 264:743-750, 1999).

Animals were euthanized at 7, 14, and 21 days. MATRIGEL®-injected and control tissues were embedded, snap-frozen in isopentane, and stored at −70° C. before sectioning with immunohistochemical staining (Sundberg, C., et al. Am. J. Pathol. 158:1145-1160, 2001).

Immunohistology, Double Immunofluorescence Staining, and Confocal Microscopy

Immunobistology, double immunofluorescence staining, and confocal microscopy were performed exactly as previously described (Sundberg, C., et al. Am. J. Pathol. 158:1145-1160, 2001).

NTPDase Activity and FACS Analysis

Monocyte/macrophages were harvested from the peritoneal cavity of wild-type and cd39-null mice after injection of 10 mL of PBS. Membrane-bound NTPDase activity was determined by measuring the amount of liberated inorganic phosphate (Pᵢ) hydrolyzed from exogenous ATP (Sigma) (Baykov, A. A., et al. Anal. Biochem. 171:266-270, 1988; Goepfert, C., et al. Mol. Med. 6:591-603,2000); 5 mmol/L tetramisole was added to inhibit alkaline phosphatase.

After isolation, naive peritoneal macrophages were also stimulated for 30 minutes (or for 24 hours) in RPMI+5% FCS, both at 37° C., with ATP 200 μmol/L, or with ATPγS 200 μmol/L, and then washed with PBS. Nonspecific binding of isospecific IgG was controlled by incubation with isospecific IgG conjugated to FITC and phycoerythrin, respectively. Cells were double-stained with phycoerythrin-conjugated rat anti-mouse CD11b in conjunction with FITC-labeled anti-mouse intercellular adhesion molecule (ICAM)-1 (or very late antigen-1, -4), respectively. Fluorescence-activated cell sorter (FACS) analysis was performed (FACSCAN™, Becton Dickinson). Data were analyzed with CELLQUEST™ computer software (Becton Dickinson).

Transmigration Assays

Transmigration assays through an endothelial cell monolayer were performed (Jones, G. E. J. Leukoc. Biol. 68:593-602, 2000). Murine 2F2B endothelial cells (ATCC) were plated on collagen type 1-coated polycarbonate inserts with 3-μm pores (Costar, Fisher Scientific) and then incubated in RPMI containing 5% FCS overnight to attain full confluence. Wild-type or cd39-null peritoneal macrophages were added to the upper chamber and incubated at 37° C. in 5% CO₂ for 10 hours. In the lower chamber, culture media with chemoattractant factors at various combinations were added: ATP 200 μmol/L, serotonin 20 μmol/L, and monocyte chemoattractant factor-1 (MCP-1) 50 ng/mL. Nonadherent cells in both supernatant fluids were counted. In parallel experiments, inserts were precoated with collagen or MATRIGEL® alone. Wild-type and cd39-null macrophages were plated on the upper surface of coated inserts. Cells were allowed to adhere and migrate over a 6-hour period. Cells on the lower surface were fixed in 2% ethanol and stained with 0.2% crystal violet (Sigma). Four fields of vision (×400) per well were randomly chosen, and cell counts were performed manually by 2 observers in a blinded fashion.

Statistical Analyses

Student's t test (2-tailed) was used for the comparative analyses.

### EXAMPLE 2

Angiogenesis Impairment in cd39-Null Mice Bearing Tumors

Tumor Cell Inoculation

The murine tumor cell lines are:
a) Lewis lung carcinoma (LLC) (ATCC Number: CRL-1642: Tumorigenic in C57 BL6 mice; Nature vol 390, 27 Nov. 1997; BLOOD, 15 Nov. 2000, Volume 96, Number 10).
b) B16-F10 melanoma (ATCC Number: CRL-6457: Tumorigenic in C57 BL6 mice; Nature vol 390, 27 Nov. 1997; BLOOD, 15 Nov. 2000, Volume 96, Number 10).
c) B16-CG melanoma (STRATAGENE Catalog #240046; The B16-CG mouse melanoma is an adherent B16 cell line stably transfected with the pVSneo-hCG plasmid. The growth medium is DMEM with G418 (250 ug/ml); Nature Medicine vol 6, 6 Jun. 2000).
   Tumor Implantation
   (1) Abdominal Wall Flap Angiogenesis Assay:

A triangular abdominal wall flap is fashioned in anesthetized mice. A subcutaneous area far from the edge of the flap is selected for the injection site. Under a dissecting microscope, 5.0×10⁵ tumor cells in 0.1 mL PBS is injected in this area using a 26-gauge needle.

At 2-day intervals, the site of tumor cell injection is examined by microscopy. The mice are anesthetized and the inner aspect of the cutaneous flap is examined for tumor growth and formation of new blood vessels around the tumor. Subperitoneal implantation is preferred as it is more physiological as compared with intradermal implantation.

(2) Subcutaneous Injection of Tumor Cells.

Tumor cells (3×10⁶ tumor cells in 0.1 ml PBS) are injected subcutaneously into mice using a 26-gauge needle. The tumor cells are injected into the dorsal subcutaneous space. Tumors are measured in two dimensions every day and the volume was calculated using the following formula: V=L·W²·π/6 (V: volume, L: longest diameter, W: shortest diameter, π: the circular constant).

(3) Injection into Systemic Circulation via Inferior Vena Cava.

LLC tumor cells at 1×10⁵ cells in 0.1 ml PBS per 25 grams body weight are injected into the systemic circulation of mice using a 26-gauge needle. B16-F10 cells and B16-CG cells are injected into mice at 1.5×10⁵ cells in 0.1 ml PBS per 25 grams body weight using a 26-gauge needle. Mice are observed daily and if undue suffering is observed, they are euthanized. Otherwise, the tumor bearing mice will be killed 15 (B16-F10 and B16-CG) or 17 (LLC) days after tumor cell injection, and the lungs harvested for immunopathology and standard evaluation of tumor size and number.

Results

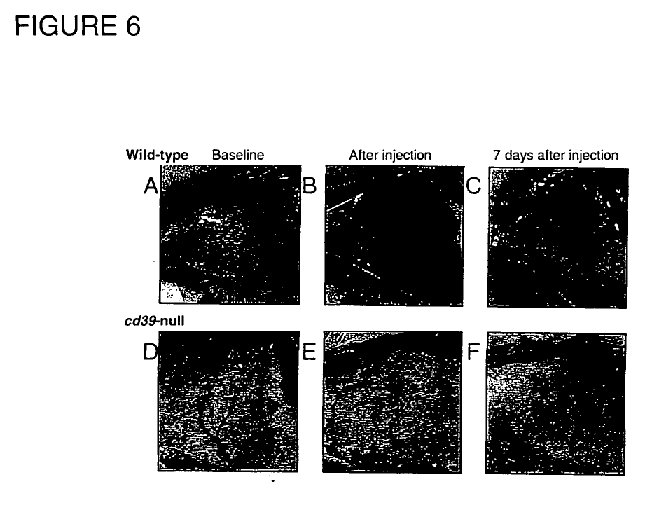

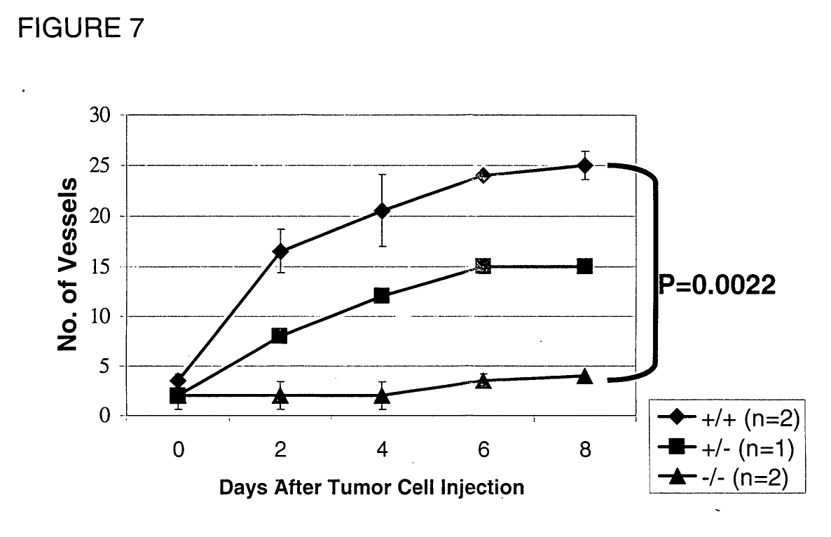

Mice were examined seven days after subperitoneal injection of B16-F10 tumor cells. Tumor size in the WT mouse was clearly larger than that observed in the cd39 knockout mouse (FIGS. 6A and 6B). In addition, the absence of CD39 in the cd39 knockout mouse resulted in a loss of neovascularization, clearly obvious in the WT mouse. This suggests that cd39 is responsible for promoting angiogenesis in the WT mouse, whereas the absence of cd39 reduces the formation of new blood vessels. FIG. 7 demonstrates that the loss of one or both copies of cd39 results in a reduction in the formation of new blood vessels. The reduction in the number of new blood vessels is reduced to a greater extent in the cd39 knockout mouse.

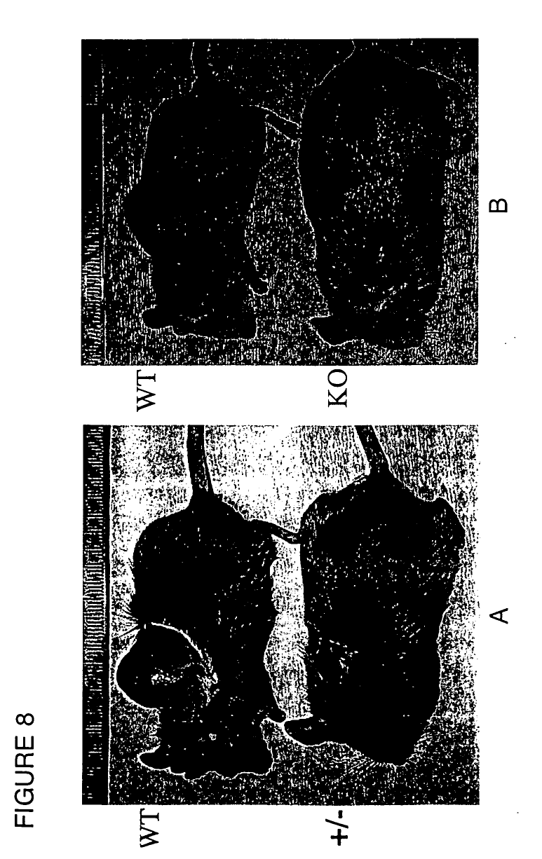

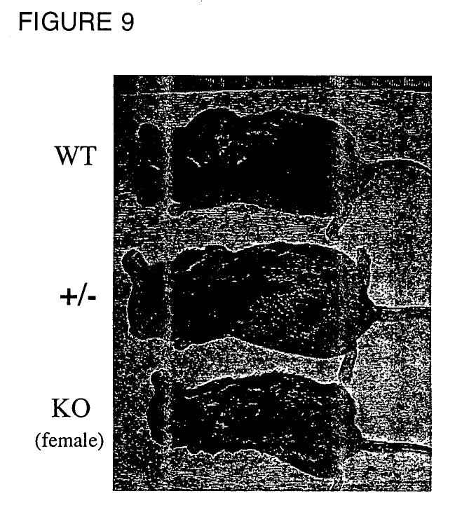

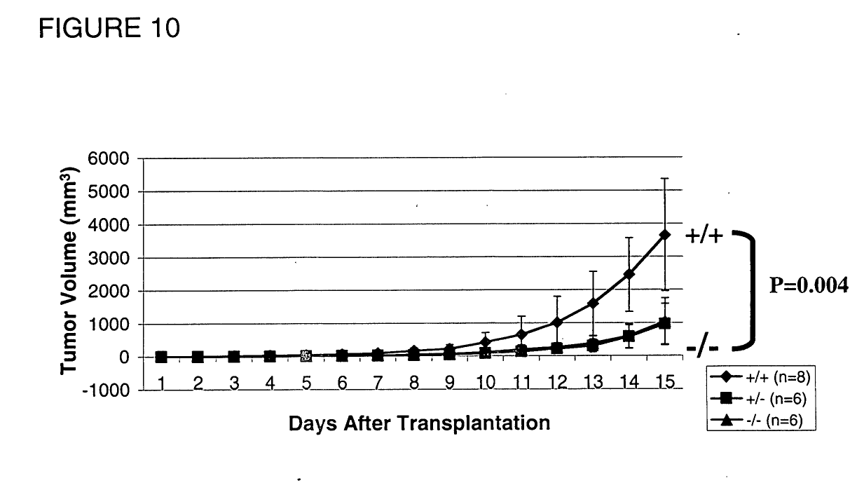

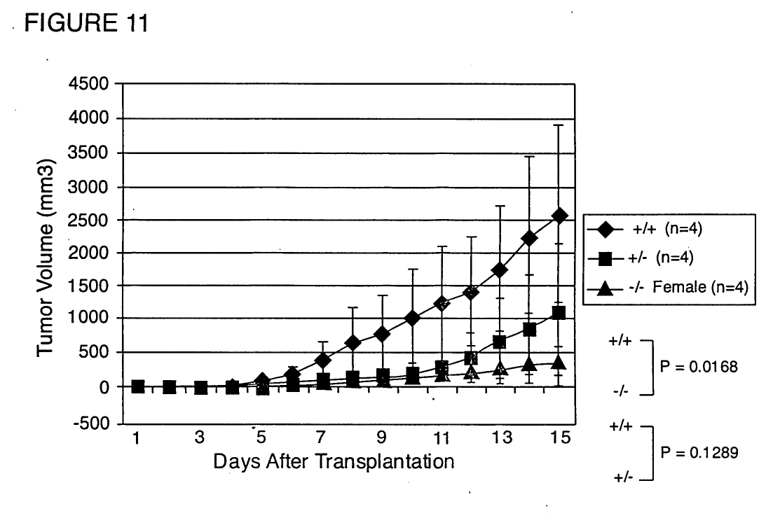

cd39 heterozygote and knockout mice were also examined for the ability to support angiogenesis in the presence of subcutaneously transplanted tumor cells (either B16-F10 or LLC). FIGS. 8 (A and B) and FIG. 9 demonstrate that the loss of one or both copies of cd39 results in a loss of angiogenesis. Tumor growth in the cd39 heterozygote and the cd39 knockout mouse is severely reduced. A comparison of the growth rate of subcutaneously injected tumors in WT mice versus cd39 heterozygotes or cd39 knockout mice (FIGS. 10 and 11) reveals that the tumors fail to attain the level of growth observed in WT mice.

The effect of CD39 on promoting tumor cell metastasis was also examined using a cd39 heterozygote and a cd39 knockout mouse. B16-F10 tumor cells were injected into the mice via the inferior vena cava. Tumor bearing mice were killed and the lungs harvested and examined for evaluation of tumor size and number. Again, only the WT mouse demonstrated an obvious ability to support tumor metastasis. Both the cd39 heterozygote and the cd39 knockout mouse exhibited fewer and smaller tumors than the WT mouse.

The data indicate that CD39 is responsible for promoting angiogenesis. The loss of CD39 results in a reduction in tumor cell formation and growth. Furthermore, the distribution of cell-type specific markers and basal lamina components in wild type versus cd39 deficient mice implanted with murine melanoma tumors indicates that CD39 is responsible for promoting angiogenesis (Table 2).

TABLE II
Distribution of Cell-Type Specific Markers and Basal Lamina Components
in Wild Type Versus CD39 Deficient Mice Implanted with Murine Melanoma
Tumors Subcutaneously

| | Adjacent | | Viable | | |
|---|---|---|---|---|---|
| | Normal | Interface | Zone | Necrosis | Capsule |
| Endothelium | | | | | |
| PECAM-1 | | | | | |
| Wild-type | + | + | + | − | + |
| Null | + | −/+ | − | − | − |
| VEGFR2-receptor | | | | | |
| Wild-type | −/+ | + | + | − | −/+ |
| Null | −/+ | −/+ | − | − | − |
| Pericytes/myofibroblasts | | | | | |
| α-Smooth muscle actin | | | | | |
| Wild-type | + | −/+ | −/+ | − | −/+ |
| Null | + | −/+ | − | − | −/+ |
| PDGF-β receptors | | | | | |
| Wild-type | −/+ | + | + | − | −/+ |
| Null | −/+ | + | − | − | −/+ |
| NG2 | | | | | |
| Wild-type | −/+ | + | −/+? | − | −/+ |
| Null | −/+ | + | −/+? | − | −/+ |
| Basal lamina (vessel associated) | | | | | |
| Perlecan | | | | | |
| Wild-type | + | −/+ | −/+*1 | − | −/+ |
| Null | + | + | −/+*2 | − | −/+ |
| Fibrin (vessel associated) | | | | | |
| Wild-type | − | + | +*1 | + | −/+ |
| Null | − | −/+ | −/+ | + | −/+ |
| Inflammatory Cells | | | | | |
| F4/80 (macrophage marker) | | | | | |
| Wild-type | −/+ | −/+ | −/+ | + | −/+ |
| Null | −/+ | −/+ | −/+ | −/+ | −/+ |
| CD41 | | | | | |
| Wild-type | | −/+ | | + | |
| Null | | | + | | + |
| B-cells | | | | | |
| Wild-type | −/+ | − | − | − | − |
| Null | −/+ | − | − | − | − |
| T-cells | | | | | |
| Wild-type | −/+ | − | − | − | − |
| Null | −/+ | − | − | − | − |
| Granulocytes | | | | | |
| Wild-type | −/+ | − | −/+ | −/+ | − |
| Null | −/+ | − | −/+ | −/+ | − |

0 indicates no expression; −/+, sporadic expression; +, abundant expression; and NA, not applicable.

### EXAMPLE 3

The efficacy of using antisense-mediated inhibition of CD39 as a therapeutic has also been demonstrated by Imai et al., Biochem. 38:13473 (1999), which is herein incorporated fully by reference.

Antisense CD39 Decreases ATPDase Activity

The use of antisense CD39, previously disclosed in Imai et al., 38:13473-13479 (1999), and incorporated herein fully by reference, has been successfully used to modulate CD39 biological activity. Antisense CD39 was used as a specific inhibitory reagent to confirm the unique biological effects of this vascular ecto-enzyme. As there are currently no specific biochemical inhibitors for ATPDase, CD39 antisense oligonucleotides, complementary to a sequence that includes the translation start site, were generated. Suppression of ATPDase activity using antisense CD39 was associated with substantive changes in levels of extracellular ATP following endothelial cell (EC) activation in vitro. These results demonstrate the efficacy of using antisense CD39 to mediate putative alterations in purinergic signaling within the vasculature and further supports the use of antisense CD39 for the treatment, prevention, or stabilization of diseases associated with the vasculature.

Experimental Procedures

Selection of Target mRNA Sequence.

A specific second generation chimeric oligonucleotide 5′-CTC CTT TGT ATC TTC CAT AAG TAG C-3′ and a scrambled control oligonucleotide 5′-TTC GTA TCT TCG CTA GCT TAT ACA C-3′ were designed and purchased from Oligos Etc./Oligo Therapeutics, Inc. (Wilsonville, Oreg.). All studied oligonucleotides have four components: an RNase H activating region; a complementarity zone; 5′ and 3′ ends. Oligomer design was optimized for thermostability, minimum self-complementarily and dimer formation. Sequences tested included the initiation start sequence (ATG) and were designed so as not to interact with CD39L1 (29). The target site of the antisense oligonucleotide, with the comparable regions of CD39L1, are indicated in Table 1.

Cell culture and use of Oligonucleotides.

Human umbilical vein endothelial cells (HUVEC) were provided by Dr. B. M; Ewenstein (Brigham and Women's Hospital, Boston, Mass.) and were used at the third passage. HUVEC were grown to 60-80% confluence on 6 or 10 cm culture plates (Nalge Nunc International, Naperville, Ill.) with gelatin-coated (SIGMA, St. Louis, Mo.) with medium 199 (BIO WHITTAKER, Walkersville, Md.) supplemented with 20% fetal bovine serum (ATRANTA biologicals, Norcross, Ga.), 50 μg/ml epidermal mitogen (Biomedical Technologies Inc., Stoughton, Mass.), 25 units/ml heparin (SIGMA ), 50 units/ml Penicillin G sodium (GIBCO BRL; Life Technologies, Inc., Grand Island, N.Y.), 50 μg/ml streptomycin sulfate (GIBCO BRL), and 2 mM L-Glutamine (GIBCO BRL). HUVEC were washed twice with Opti-MEM (GIBCO BRL) prewarmed to 37° C. Opti-MEM containing 6 μM LIPOFECTIN® Reagent (GIBCO BRL), 1:1 (w/w) liposome formulation of the cationic lipid N-[1-(2,3-dioleyloxy)propyl]-n,n,n-trimethylammonium chloride (DOTMA), and dioleoyl phosphotidylethanolamine (DOPE) in membrane filtered water, were pre-incubated at room temperature for 30-45 min and then added to the HUVEC. Oligonucleotides at a final concentration of 4 μM were incubated with HUVEC for 4 h at 37° C. Medium was removed and the cells were washed gently with Opti-MEM and replaced with the complete growth medium. Cells were then cultured for further 44 h at 37° C. and assayed.

In parallel, COS-7 cells were cultured and transfected with antisense/scrambled control oligonucleotides in the identical manner, 24 h following initial exposure to pCR II or pCRII-CD39 cDNA (25).

Localization of Antisense Oligonucleotides.

HUVEC were grown on human fibronectin-coated 2-well culture slide (Becton Dickinson Labware, Franklin Lakes, N.J.) and treated with 3′ FITC-labeled oligonucleotides (Oligos Etc. Inc.), as described above. After 44 h, cells were washed with DMEM (GIBCO BRL) for 1 min and fixed with 4% paraformaldehyde in DMEM for 5 min at room temperature. After fixation, cells were washed with DMEM for 2 min and dehydrated in a graded series of alcohol 70%-100% for 1 min each. The subcellular localization of the FITC-labeled oligonucleotides was determined, using a OLYMPUS BX40 fluorescent microscope (OLYMPUS, Japan).

Intracellular Preparation of RNA and Northern Blotting.

Total RNA was isolated from cells by the method of Chomczynski and Sacchi (30). RNA samples (20 μg per well) were separated on 1.0% agarose gel containing 10% formaldehyde, and then transferred to a nylon membrane (Hybond-N+; Amersham Life Science Inc., Arlington Heights, Ill.). Integrity, consistency of loading, and transfer of RNA were verified by ethidium bromide staining of ribosomal RNA and by analysis of glyceraldehyde-3-phosphate dehydrogenase (GAPDH) mRNA levels. CD39 cDNA (1714 base pairs fragment) was used as probe and GAPDH was used as an internal control probe (25). These were labeled with [α-³²P] dATP using Ready-To-Go labeling kit (Pharmacia Biotec Inc., Uppsala, Sweden). Prehybridization, hybridization, and washes of the membrane were carried out according to the rapid hybridization protocol from Stratagene (La Jolla, Calif.). Final washes were at 55° C. in 0.5 (sodium saline citrate/0.1% SDS for 30 mn. The blots were exposed to Kodak Biomax MR film (Eastman Kodak Co.) with intensifying screens for 1 day at −80° C.

RNA stability

HUVEC were incubated with 0.25 μg/ml of α-amanitin (Boehringer Mannheim GmbH, Germany) for 0, 1, 2, 8, 16, 24 and 48 h. RNA was isolated and analyzed by Northern hybridization with CD39 and GAPDH cDNA as described earlier (25).

Cell Lysate Preparation and Western Blotting.

After a 44 h incubation (and following the 4 h transfection), EC were washed twice with 20 mM Tris-saline buffer, pH 8.0 at 4° C. on ice, harvested by scraping in Tris-saline containing 0.04 TIU/ml Aprotinin (SIGMA) and centrifuged 800 g for 15 min at 4° C. Cells were resuspended in Tris-saline, 0.04 TIU/ml Aprotinin, 1% NONIDENT® P 40 (Fluka, Ronkonkoma, N.Y.), and 0.1 mM phenylmethylsulfonylfluoride. This mixture was incubated for 20 min on ice and centrifuge 16,000 g for 20 min at 4° C. Supernatants containing cell lysate were used for Western blot analysis and ATPDase activity assay. Protein concentrations were measured using Bio-Rad Dc Protein Assay (Bio-Rad Laboratories, Hercules, Calif.) and read at 750 nm on a microplate spectrophotometer (EL 340; Bio-Tek Instruments, Inc., Winooski, Vt.). Proteins (10 μg per lane) were fractionated on a 10% SDS-polyacrylamide gel under non-reducing conditions according to Laemmli (31), transferred to polyvinylidene difluoride membrane (Immobilon P; Millipore, Bedford, Mass.) by semi-dry electroblotting and then probed with commercial monoclonal antibodies (mAb) to CD39 (Accurate, Westbury, N.Y.). Bands were visualized using horseradish peroxidase-conjugated polyclonal goat anti-mouse IgG (Pierce, Rockford, Ill.) and the Western Blot Chemiluminescence Reagent Plus (NEN™ Life Science Products, Boston, Mass.) according to the manufacturer's instructions.

ATPDase Activity Assay.

Ca⁺⁺, Mg⁺⁺-dependent ATPDase activity was determined by measuring inorganic phosphate (Pi) release from ATP or ADP in the presence of 5 mM tetramisole (SIGMA) to eliminate effects of contaminating alkaline phosphatases. Enzyme activity was determined at 37° C. in 1 ml of: 5 mM CaCl2, 200 μM substrate (ATP or ADP) and 50 mM Tris, pH 8.0 as previously described (32). Reactions were stopped with 0.25 ml of malachite green reagent, and inorganic phosphate (Pi) was estimated according to Baykov (33). One unit of ATPDase activity corresponds to the release of 1 μmol Pi/min at 37° C. Absorbance was read at 610 nm on a microplate spectrophotometer. Experiments were repeated with different HUVEC cultures in all cases.

Measurement of ATP Release.

Transfected or control HUVEC were incubated with DMEM supplemented with 15% fetal bovine serum, 50 units/ml Penicillin G sodium, 50 μg/ml streptomycin sulfate, 2 mM L-Glutamine, and 100 μM adenosine (Adenocard; Fujisawa, Japan) for 44 h after transfection. At the end of incubation, culture medium was changed to DMEM with 10 μM ADP (SIGMA) to stimulate HUVEC, and samples of conditioned medium were collected at pre-determined intervals (0-60 min) then reconstituted in EDTA (final concentration; 2.6 mM).

ATP concentrations were assayed by modification of the method of McCall (34) using a MicroLumat LB96P luminometer (EG&G Berthold, Germany). In this procedure, ATP is detected, by the specific enzymatic reaction of firefly luciferase with luciferin (35). Briefly, 20 μl of samples or standards, with 80 μl of 25 mM diglycin (GLY-GLY SigmaUltra; SIGMA), 10 mM MgSO4 and 100 ng luciferase (SIGMA) were added to each well of 96-well immunofluor plate. In the luminometer, 20 μl of 0.2 μM luciferin (Molecular Probes, Inc., Eugene, Oreg.) was added to each well via an automated dispenser and luminescence was monitored over a 10 sec period; all measurements were done at room temperature. The responses in a given sample or standard were integrated and averaged for several determinations. Data are expressed as μM ATP derived from standards examined under the same conditions and plotted over the identical time period examined. Corrected ATP concentrations were then calculated by subtracting any contaminants of ATP in the commercial proportions of ADP. Total ATP released by EC was estimated by measurements of the relevant area under curve of kinetic plots and the rates of the initial hydrolysis of ATP were directly calculated.

Statistical Analysis

The statistical analysis was performed by a two tailed paired t test for comparisons with the control group (Microsoft Excel 5; Microsoft). Differences were considered significant when p<0.05.

Effect of the Antisense Oligonucleotides on CD39 mRNA Expression

To assess whether oligonucleotides were able to modify levels of CD39 RNA, we performed Northern blotting after transfection using CD39 cDNA (25) as the probe. Anti sense oligonucleotides specifically inhibited transcription of CD39 mRNA, when compared with scrambled oligonucleotides and control liposomes at 48 h post-transfection (see FIG. 3, Imai et al., 38:13473-13479 (1999)). We detected three different mRNA transcripts specific for CD39 as previously described (25); all bands were decreased by the specific antisense oligonucleotide treatments.

Effects of the antisense oligonucleotides on CD39 protein expression.

To determine the effect of the decreased RNA levels on CD39 translation, we then studied protein expression by Western blotting in parallel with the Northern analyses. Representative cultures of HUVEC treated with 6 μM ATPDase/CD39 antisense oligonucleotides had decreases in immunoreactive intact CD39 (to 66%), when compared by direct densitometric analysis with control HUVEC exposed to scrambled oligonucleotides (103%) or control liposomes (100%); all cultures were studied at 48 h after transfection (see FIG. 4, Imai et al., 38:13473-13479 (1999)). No alterations in the ratio of intact to the 56 kDa proteolytic isoform (11) were observed in more exposed autoradiographs (data not shown).

Effect of the Antisense Oligonucleotides on ATPDase Activity.

HUVEC and COS-7 cell lysate preparations were incubated with exogenous ADP or ATP to determine ATPDase activity. Specific ADPase activity for HUVEC was 9.5±3.4 nmole Pi/min/mg protein (mean (SD; n=6) for HUVEC transfected with antisense oligomers; 22.6±2.1 with scrambled control oligomers, and 26.0±3.1 for control liposomes. ATPase activity was 15.1±3.4 nmole Pi/min/mg protein with antisense oligomers; 29.1±1.2 with scrambled control oligomers, and 28.2±4.6 for control liposomes (see FIG. 5, Imai et al., 38:13473-13479 (1999)). Normalized ADPase activity was significantly inhibited (to 42%) by antisense oligomers when compared with EC incubated with scrambled oligomers (p<0.005; n=6) or to 36% of control liposomes (p<0.005; n=6). Antisense oligomers inhibited ATPase activity to 51% of that for scrambled control oligomers (p<0.005; n=6) and to 53% of control levels (p<0.005; n=6). Suppression of ATPDase activity was not due to a toxic effect of the treatment as cell viability determined by trypan blue exclusion remained over 90%. Total cell protein content from antisense oligonucleotide transfected HUVEC (17.6±2.8 (g/10⁶cells, mean±SD; n=6), scrambled control oligonucleotides transfected HUVEC (17.7±1.8) and control cells (17.9±5.7) were also comparable (data not shown).

In parallel, ATPDase activity was assayed in COS-7 cells, transfected sequentially with CD39 cDNA and oligonucleotides. Antisense oligonucleotides decreased ADPase levels to 15% and ATPase to approximately 5% of control cells transfected with CD39 cDNA (data not shown).

Effect of the Antisense Oligonucleotides on ATP Release by Stimulated HUVEC.

We evaluated the effects of suppression of ATPDase activity by antisense oligonucleotides on ATP release from ADP-stimulated HUVEC. Rapid induction of ATP-release by purinergic stimulated-HUVEC was observed. Peak concentrations of extracellular ATP were comparable in the control and oligonucleotide treated HUVEC cultures. Antisense oligonucleotide treatment also boosted area under curve estimates of ATP release by 194% (over first 15 min), when compared to controls. Rapid degradation of ATP was observed in minutes in control HUVEC cultures. Treatment with the antisense oligonucleotide prolonged elevated ATP extracellular concentrations (see FIG. 6, Imai et al., 38:13473-13479 (1999)). Initial elimination rates of extracellular ATP by hydrolysis were decreased by antisense oligonucleotides in keeping with the suppression of CD39 expression. The rate of ATP hydrolysis by EC following transfection with antisense oligonucleotides was 4.8 (0.5 pmole/min/10⁶cells (mean (SD; n=9); using scrambled oligomers was 19.6 (0.1 pmole/min/10⁶cells and liposome treated controls 17.9 (5.0 pmole/min/10⁶cells.

Substantial suppression of ATPDase/CD39 activity was achieved in both EC and transfected COS-7 cells following treatment with antisense oligomers in vitro. Such decreases in levels of expressed ATPDase activity were demonstrated to have potential biological effects with respect to alterations in concentration of extracellular adenine nucleotides in vitro. Although initial levels of ATP secreted by activated EC were comparable in both control and treated cells, substantive delays in hydrolysis of extracellular ATP were observed following the use of antisense oligomers to CD39 in keeping with the suppression of ATPDase expression. Comparable changes in the purinergic signaling environment could have major implications for the evolution of vascular activation responses in vivo.

Exogenous adenosine was added to EC cultures to provide adequate levels of intracellular ATP for the stimulation experiments. Identical ATP release profiles were observed in all cell cultures, irrespective of levels of CD39 expression and indicative of adequate ATP storage despite antisense treatments. The major differences observed in antisense oligomer treated EC were that high levels of released ATP persisted in that the kinetics of elimination were retarded by suppression of CD39 expression. Associated delays in extracellular adenosine formation and uptake would likely ensue.

Substantive decreases in the levels of specific RNA transcripts was observed when tested at 48 h. The effects of the antisense oligonucleotide on CD39 protein expression, as determined by qualitative Western blotting were less dramatic with approximately 40% apparent decreases observed (see FIG. 4, Imai et al., 38:13473-13479 (1999)). Previous work by us and others have demonstrated that the presence of immunoreactive CD39 antigen alone does not correlate with functional ATPDase activity; the formation of multimers (49) or oxidative inactivation are added factors that influence enzymatic activity (11). Experiments consistently demonstrated that the bulk of EC ecto-ADPase activity could be inhibited by antisense oligomers directed at CD39 (see FIG. 5, Imai et al., 38:13473-13479 (1999)). Less substantive and relative decreases in ecto-ATPase levels may be in keeping with prior observations of the co-expression of homologues of CD39 such as CD39L1 on EC (38) 4. The modest decrease in CD39-immunoreactivity also did not directly correlate with the substantial simultaneous decreases in specific expression of ATPDase activity and suggested that the persistent expression of immunoreactive CD39 protein did not indicate the full potential for biochemical activity. In addition, no increase in proteolytic degradation to the 56 kDa isoform (49) were observed. In COS-7 cells, suppression of new CD39 synthesis by antisense oligonucleotides was far more efficient and keeping with contribution of newly synthesized CD39 to the cellular ATPDase activity (data not shown). All these findings are consistent with our published observations that decreases in ATPDase activity in vascular EC may occur without proteolytic degradation or immediate loss from the plasma membrane and suggest that functional inactivation of CD39 may occur without loss of cellular immunoreactivity (11).

We have already established that vascular ATPDase/CD39 expression may be rapidly decreased by reperfusion injury (8), oxidant stress (12, 25) or cytokine mediated EC activation responses (11). Our data suggest that loss of ATPDase activity, following suppression of CD39 synthesis may result in decreases in extracellular adenine nucleotide hydrolysis. In the current study, we show that substantial loss of specific ATPDase activity results in persistent elevations in concentrations of ATP in the extracellular environment.

### EXAMPLE 4

Targeted Delivery of CD39

The efficacy of using adenoviral-mediated delivery of CD39 as a therapeutic has also been demonstrated by Gangadharan et al., Surgery 130: 296-303 (2001), which is herein incorporated fully by reference. An adenoviral construct containing the human CD39 gene (Ad-CD39), under control of the cytomegalovirus immediate-early promoter and an SV40 polyadenylation sequence was employed in these studies (Kaczmarek et al., (1996); Imai et al., (2000)). The control vector (Ad-LacZ) contained the Escherichia coli marker gene-galactosidase, also driven by the cytomegalovirus promoter. The recombinant vectors were E1, E3-deleted, serotype 5, with the transgene cassette inserted into the E1 region of the genome. Adenovirus vector preparations were provided by the Vector Core Laboratory of the Harvard Institute for Human Genetics.

In a cardiac xenotransplant model, adenoviral mediated transfer of the human CD39 gene resulted in prolonged graft survival that was associated with increased NTPDase activity.

In Vitro Gene Transfer

Early passage smooth muscle cells (SMC) or endothelial cells (EC) isolated from human saphenous vein were grown to near-confluence in complete medium. Preliminary in-vitro studies were performed to evaluate CD39 gene transfer to vascular target cells. Ad-CD39 and Ad-LacZ infections were performed in serum-free medium at a multiplicity of infection (MOI) of 103 plaque-forming units (pfu)/cell for 0.5 hr at 37° C. Untreated controls were also processed. After incubation, the cells were washed twice, then returned to complete medium. Four days later, the cells were harvested for measurement of NTPDase activity.

Cell Lysate Preparation

Transduced cells were harvested in Tris-saline buffer pH 8.0 containing aprotinin (10 μg/ml, Sigma). Cell lysis was performed in Tris-saline buffer pH 8.0, with aprotinin, 1% NP40 (Sigma), and 100 μM phenylmethylsulfonylfluoride (PMSF, Sigma). The supernatant was stored at −80° C. for subsequent determination of NTPDase activity.

Arterial Injury Model and in Vivo Gene Transfer

Adult New Zealand white rabbits (n=28) weighing between 3 and 4 kg were given ad libitum access to water and chow. The animal experimentation protocol was reviewed and approved by the Harvard Medical Area Standing Committee on Animals. Animal care and procedures were carried out in accordance with the Guide for the Care and Use of Laboratory Animals (Institute of Laboratory Animal Resources, Commission on Life Sciences, National Research Council, Washington: National Academy Press, 1996). Animals were anesthetized with ketamine (25 mg/kg, Fort Dodge Laboratories, Fort Dodge, Iowa) and xylazine (5 mg/kg, Fermenta Animal Health Co., Kansas City, Mo.). Flunixamine (Fort Dodge) was given intramuscularly for post-operative pain control.

An established model of bilateral balloon injury of the iliofemoral arteries was used.23, 24 In brief, the ileofemoral arteries were exposed and surgically isolated. A 2-French balloon embolectomy catheter (Baxter Healthcare Corp., Deerfield, Ill.) was used to injure the vessels. Paired experiments were performed for direct comparison of treated and control arteries. Initial studies examined the effects of balloon injury on vessel NTPDase activity in a subset of animals (n=5) who underwent unilateral balloon injury, with the contralateral iliac artery surgically isolated, but left uninjured. All subsequent gene transfer experiments were performed following bilateral balloon arterial injury. One iliac artery was incubated for 0.5 hr with 10¹⁰ pfu/ml of Ad-CD39. The contralateral artery received either vehicle (PBS; n=10) or 10¹⁰ pfu/ml of Ad-LacZ (n=9). Following incubation the vessel contents were aspirated and flow restored.

At sacrifice, 2 days after injury, heparin (1000 units) was given intravenously, the animal was sacrificed, and the arterial tree was flushed with normal saline. The iliofemoral segments were excised, and frozen specimens were obtained for immunohistochemistry and determination of NTPDase activity. In a subset of animals (n=6) treated with Ad-CD39 versus Ad-LacZ, the entire vessel was processed for platelet deposition studies as described below.

Vessel Homogenate Preparation and Determination of NTPDase Activity

Arterial specimens were harvested and tissue homogenates were prepared in buffer containing Tris-saline pH 8.0, aprotinin, and PMSF. Vessel homogenates or cell lysates were incubated with 200 μM substrate (ADP or ATP), and Ca⁺⁺-dependent release of free phosphate was determined, as previously described (Sevigny et al., (1995)). Malachite green was added to stop the reaction, and absorbance was measured at 610 nm to determine levels of phosphate generation against the standard curve of KH₂PO₄. Protein was measured according to the Bradford method (Bradford, M. M. (1976)).

Immunohistochemistry

Snap frozen specimens were cut at 6-μm thickness. Tissue was fixed in acetone at 4° C. for 10 minutes, quenched in 0.3% H₂O₂, and blocked with 10% normal horse serum. Anti-CD39 primary antibody (BU61, 1:500, mouse anti-human, Ancell Corp., Bayport, Minn.) was applied for 1 hr at 22° C. Non-specific IgG or PBS controls were performed on each slide. The specimen was then rinsed and the biotinylated secondary antibody (1:50 horse anti-mouse, Vector Laboratories, Inc., Burlingame, Calif.) was applied. Visualization of primary antibody binding was accomplished using an avidin-biotin-peroxidase complex (ABC Elite Kit, Vector) followed by exposure to Novared(tm) (Vector). Sections were counterstained with Gill's hematoxylin.

Statistics

All values were compared by using a paired, two-tailed t-test. Statistical significance was based on p value<0.05.

Effect of Balloon Injury on Vessel NTPDase Activity

Balloon injury resulted in a decrease in both ADPase and ATPase activity of the arterial wall. ADPase activity was decreased from 31.4±12.2 nmol Pi/min/mg protein in native rabbit arteries to 19.2±8.5 nmol Pi/min/mg protein two days after balloon injury (mean±SD; p<0.05). ATPase activity similarly declined, from 63.9±8.2 nmol Pi/min/mg protein in the uninjured vessel to 41.8±13.0 nmol Pi/min/mg protein after balloon injury (p<0.005). (see FIG. 1, Gangadharan et al., Surgery 130: 296-303 (2001))

NTPDase Activity of Transduced Cells in Vitro

Human saphenous vein SMC and EC exposed to Ad-CD39 demonstrated augmentation of ATPase and ADPase activity. In SMC, ADPase activity was augmented 3-fold (Ad-CD39 9.2 vs. untreated 2.9 vs. Ad-LacZ 3.3 nmol Pi/min/mg protein) and ATPase activity was doubled (Ad-CD39 16.0 vs. untreated 7.4 vs. Ad-LacZ 7.8 nmol Pi/min/mg protein). In EC, ADPase activity was increased by more than 40-fold (Ad-CD39 3429.7 vs. untreated 60.0 vs. Ad-LacZ 84.7 nmol Pi/min/mg protein). ATPase activity was similarly increased in transduced EC (Ad-CD39 4381.2 vs. untreated 102.2 vs. Ad-LacZ 127.9 nmol Pi/min/mg protein).

Localization of Transgene Expression in Vivo

Immunohistochemistry confirmed expression of the human CD39 transgene in balloon-injured arteries treated with Ad-CD39 (see FIG. 2, Gangadharan et al., Surgery 130: 296-303 (2001)). In contrast, none of the vehicle-treated or Ad-LacZ-treated vessels exhibited positive staining. The monoclonal anti-human CD39 antibody did not detect native rabbit CD39, as evidenced by the absence of staining in the uninjured rabbit vessels. Vessels treated with Ad-CD39 demonstrated a partially circumferential, luminal distribution of the transgene product, with only the innermost layers of the denuded artery staining positively. This was consistent with previous observations of luminal β-galactosidase activity after infection with Ad-LacZ in the same model.

NTPDase Activity of Vessels Following Ad-CD39 Infection

In vivo infection of balloon-injured arteries with Ad-CD39 significantly augmented ADPase and ATPase activity, when measured on vessel homogenates ex vivo. ADPase activity was 33.3±14.7 nmol Pi/min/mg protein in vehicle-treated vessels, and 47.6±21.1 nmol Pi/min/mg protein after infection with Ad-CD39 (p<0.05). ATPase activity was 59.7±23.3 nmol Pi/min/mg protein after vehicle treatment, and 90.98±46.0 nmol Pi/min/mg protein after Ad-CD39 (p<0.05; n=10; see FIG. 3a, Gangadharan et al., Surgery 130: 296-303 (2001)). Ad-CD39 similarly increased NTPDase activity when compared to Ad-LacZ. ADPase activity was 28.3±5.3 nmol Pi/min/mg protein after Ad-LacZ infection, and 60.2±17.8 nmol Pi/min/mg protein after Ad-CD39 (p=0.05). ATPase activity was 67.8±23.0 nmol Pi/min/mg protein after Ad-LacZ infection and 95.0±8.1 nmol Pi/min/mg protein after Ad-CD39 (p=0.11; n=3; see FIG. 3b, Gangadharan et al., Surgery 130: 296-303 (2001)). There was no obvious correlation between the extent of CD39 expression by immunohistochemistry and the NTPDase activity measured from tissue.

This study shows that 1) direct mechanical arterial trauma decreases vessel wall NTPDase activity and 2) adenovirus-mediated transfer of the human CD39 gene augments NTPDase activity in the balloon-injured rabbit iliac artery. The gene product is localized to the luminal surface of the vessel.

Decreased total ADPase and ATPase activity following balloon injury was an expected observation, as previous work had strongly localized vessel wall NTPDase activity to the endothelium (Cote et al. (1991), and this injury model is well known to result in complete endothelial denudation (Gangadharan et al., (2001). The importance of endothelial cell CD39 in local thromboregulation has been demonstrated in several previous studies. CD39 ADPase activity is a PGI₂- and nitric oxide-independent mechanism of endothelial cell inhibition of platelet aggregation (Kaczmarek et al. (1996); Marcus et al. (1991); Marcus et al. (1997)). Though vascular smooth muscle cells and preparations of tunica media from aortic tissue have also have been shown to exhibit NTPDase activity (Sevigny et al. (1997); Cote et al. (1992)), our data support the notion that vascular endothelium predominantly contributes to the total NTPDase activity of arteries. Therefore this represents another potential important mechanism whereby the acutely injured vessel surface may demonstrate an increased propensity for thrombosis. Previous investigations utilizing an identical recombinant adenovirus vector (Ad-CD39) demonstrated that treatment of cardiac xenografts increased NTPDase activity, decreased vascular thrombosis, and prolonged graft survival (Imai et al. (2000). The model of luminal gene transfer presented in this paper shows that NTPDase activity can be successfully augmented in segments of injured artery by Ad-CD39 exposure.

Central to the success of any gene transfer strategy is localization of expression to the relevant target cell population and production of physiologically relevant quantities of transgene product. In the setting of acute arterial injury, luminal SMCs are the appropriate target for strategies aimed at influencing cell adhesion and thrombosis, and they were efficiently transduced in this study by intraluminal administration of Ad-CD39. In vitro, augmentation of NTPDase activity by Ad-CD39 was more variable and lesser in magnitude for SMC than EC. Preliminary studies using a longer vector exposure time (overnight) have demonstrated a>30-fold increase in transduced SMC, suggesting the absence of any intrinsic defect in CD39 function in these cells. Manipulation of the vector construct (e.g., promoter) may be necessary to promote maximal CD39 expression in luminal SMC. Of note, we measured total NTPDase activity of the vessel wall, which includes potential sources within the deeper media and adventitia that would not be likely to impact on platelet aggregation. Given the localization of CD39 seen by immunohistochemisty, it seems likely that NTPDase activity on the luminal surface would be even more impressively augmented than was evident in our data.

The ability to transfer the human CD39 gene efficiently to a vessel surface in a clinically-relevant model of arterial angioplasty is an important step in the search for locally-acting therapeusis of vascular injury.

### EXAMPLE 5

In all instances examined to date, newly developing vascular endothelia express very high levels of CD39/NTPDase1, the dominant vascular ectonucleotidase, as is also seen under hypoxic conditions (Eltzschig et al., J. Experimental Med. 198:783-796, 2003). The CD39 N-terminal intracytoplasmic domain has been shown to be palmitolylated while the sequence of the C-terminus contains a putative PDZ domain (-K-D-M-V). This domain may dictate interactions with select P2 receptors, e.g., the P2Y1 receptor ends in -D-T-S-L and P2Y2 ends in -D-I-R-L (Hall et al., P.N.A.S. USA 95:8496-8501, 1998). In addition, the intracytoplasmic domains of NTPDases potentially interact with intracellular components of nucleotide-mediated or growth factor and/or insulin mediated signaling pathways that may independently modulate cellular migration and angiogenesis (Koziak et al. J. Biol. Chem. 275:2057-2062, 2000; Robson et al., Drug Dev. Res. 53:193-207,2001; Goepfert et al., Circulation 104:3109-3115, 2001; and Goepfert et al., Mol. Med. 6:591-603, 2000).

Pathological new blood vessel development is implicated in diverse conditions, inclusive of inflammation, neoplasia, transplant rejection, vascular insufficiency, and remodeling (Beck et al., FASEB J. 11:365-373, 1997). Angiogenesis requires the dynamic interaction of EC proliferation and differentiation with orchestrated interactions between extracellular matrix and surrounding cells (such as vascular smooth muscle and/or pericytes) that could be coordinated by interplay between nucleotide release, P2 receptor and integrin modulation, and NTPDase expression (Robson et al., Drug Dev. Res. 53:193-207, 2001; and Luscinskas et al., FASEB J. 8:929-938, 1994).

Our experimental systems studying angiogenesis in cd39 null mice and other models confirm that major alterations are observed with matrigel plug vascularization, tumor cell implantation, myocardial and hind limb ischemia, liver regeneration, and finally, the revascularization of transplanted organs and cells (Goepfert et al., Circulation 104:3109-3115, 2001).

In Vivo Models of Angiogenesis to Evaluate Potential Links Between NTPDases, P2 Receptors, Growth Factors, and Integrins.

Migrating monocyte-macrophages, ECs, and pericytes remained stratified at the tissue-matrigel interface in cd39 null mice where absolute failure of new vessel ingrowth growth was observed. Impaired chemotaxis of cd39 null monocytes-macrophages was associated with desensitization of nucleotide P2Y-receptors (Goepfert et al., Circulation 104:3109-3115, 2001).

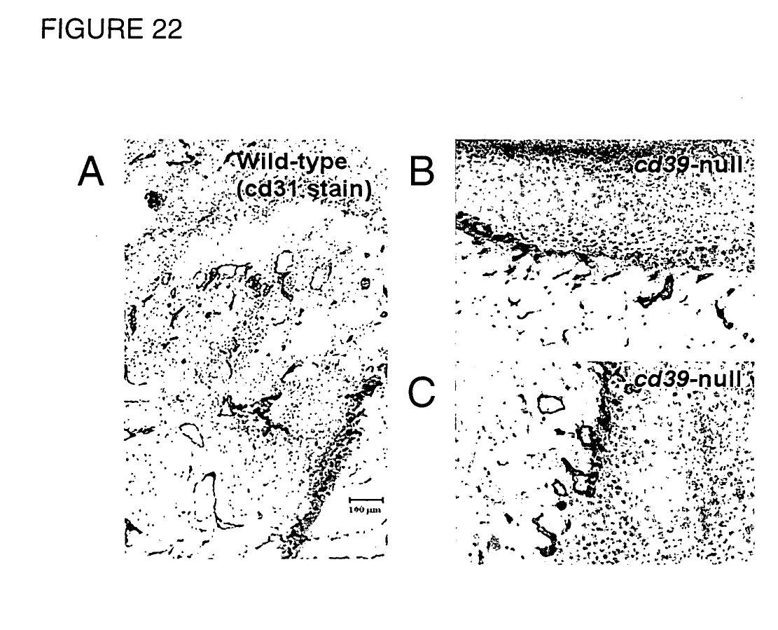

For tumor angiogenesis work, cell suspensions of syngeneic B16-F10 cells were transplanted into subperitoneal space or into the subcutaneous tissues of matched groups of 10 wild type and cd39 null mice, and analyzed at 7 and 10 days. Growth of tumor nodules and neovascularization were observed in wild type mice. In contrast, no substantive increments in tumor size were observed in the subperitoneal space of cd39 null mice, nor was there any exponential phases of tumor growth in the subcutaneous tissues. Immunohistochemical stains for the EC marker CD31 (PECAM) of the excised subcutaneous tissue tumor biopsies confirmed extensive revascularization of tumor nodules in wild type mice. In the mutant mice, vascular budding from mother vessels appeared to be preserved but the new vessels did not penetrate the tumor cell mass and remained at the periphery of the lesions (n=10; see FIGS. 6A-6F and FIGS. 22A-22C).

In other experimental systems studying angiogenesis in cd39 null mice, major and consistent alterations have been observed with myocardial and hind limb ischemia, liver regeneration, and in the revascularization of islet grafts.

In the instance of myocardial infarction models, in contrast to wild type hearts, low levels of metalloproteinase-9 expressing monocytes and EC were observed in ischemic and necrotic myocardial tissues in cd39 null mice following immunoperoxidase staining; a pattern analogous to that seen with matrigel injection (Goepfert et al., Circulation 104:3109-3115, 2001).

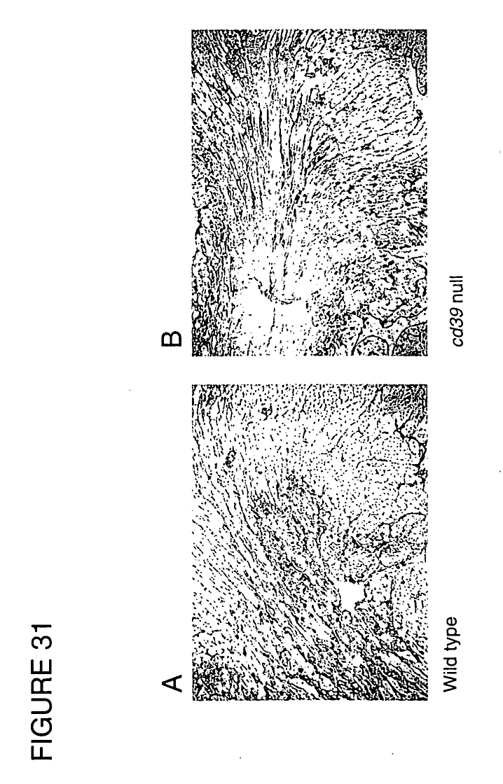

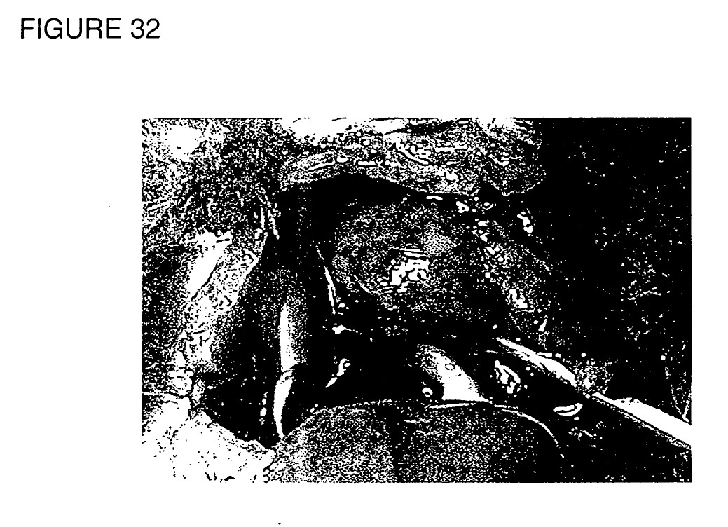

Infarct sizes following LAD ligation were comparable in both groups of mice (n=10), despite higher heart/body weight ratios in mutant mice. The pathophysiological neovascularization response in the ischemic myocardium in wild type mice was associated with infiltration of monocytes expressing matrix metalloproteinase 9 (MMP9; FIG. 31) with upregulation of cd39 with intense capillary endothelial staining. In contrast, there was failure of angiogenic responses in cd39 null mice concordant with documented defects in monocyte migration and vascularization (FIG. 3). Paradoxically, this provided an early survival advantage in cd39 null mice post-myocardial infarction by preventing early myocardial remodeling and rupture (FIG. 32). We concluded that the absence of cd39 provided early protection against myocardial ischemic injury, by blocking angiogenesis and early myocardial remodeling.

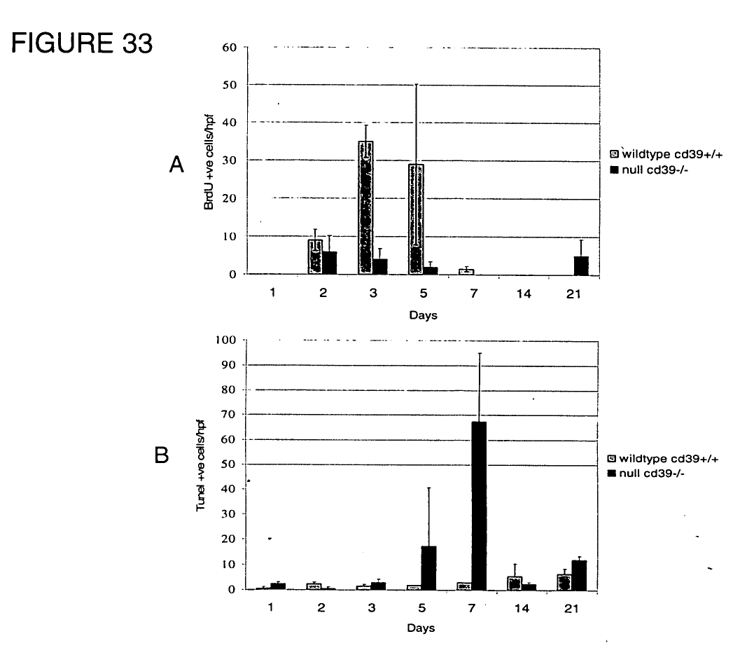

With respect to the 70% hepatic resection and regeneration models, we also observed substantial perturbations in the process of sinusoidal EC proliferation suggesting resistance to vascular growth factors with heightened levels of apoptosis observed in cd39 null hepatic vasculature (FIGS. 33A and 33B).

Nucleotide-Mediated Signaling, Adhesion and Proliferative Effects

Both cellular adhesion to matrix proteins and mitogenic signaling by growth factors are required for cellular proliferation. It has been known for some time that integrin-mediated signaling processes may synergize with growth factor responses and the engagement of the vitronectin receptor in EC-matrix interactions coordinates intense responses to both PDGF and insulin (Schneller et al., EMBO J. 16:5600-5607, 1997). Interestingly, EC also express c-Met, the receptor for hepatocyte growth factor, and proliferate markedly in response to this growth factor.

We have performed microarray experiments of total RNA from wild type and cd39 null mice from lungs and livers under basal and activated conditions using Affymetrix murine MG-U74A GeneChip®. Amongst other data, major upregulation of murine guanylate binding protein isoform 1 (GBP-1; M5544) in mutant lung was observed and validated by Northern analysis. The expression of GBP-1, a cytoplasmic protein, is selectively induced by interferon-γ, interleukin-1α, interleukin-1β, and tumor necrosis factor-α, but not by other cytokines, chemokines, or growth factors (Lubeseder-Martellato et al., Amer. J. of Pathol. 161:1749-1759, 2002). GBP-1 expression is highly associated with vascular EC and belongs to a special class of large GTP-binding proteins of 60-100 kDa (Prakash et al., EMBO J. 19:4555-4564, 2000). This protein may influence growth factor/insulin responses and the process of angiogenesis (Guenzi et al., EMBO J. 22:3772-3782, 2003; and Guenzi et al., EMBO J. 20:5568-5577, 2001). Some of the signaling molecules associated with tyrosine kinase growth factor receptors, such as Src and phosphatidyl inositol 3 kinase (PI3K), are also present in integrin complexes at focal adhesions (Weisman et al., Drug. Dev. Res. 53:186-192, 2001). Cells require integrin-mediated attachment to extracellular matrix to be able to respond to growth factor stimulation. In addition, growth factors often induce cell migration, which is an integrin-dependent process (Schneller et al., EMBO J. 16:5600-5607, 1997). Activation of P2Y2 by ATP or UTP is known to cause directed cell migration, although the cellular events leading to this process in angiogenesis are incompletely understood (Seye et al., Circulation 106:2720-2726, 2002; and Liu et al., J. Biol. Chem. 279:8212-8218, 2004).

EC proliferative responses are also crucial in angiogenesis. It is well established that progression through the cell cycle is regulated by sequential phosphorylation with accumulation of cyclins, regulatory cyclin-dependent kinases (CDKs), and inhibitors of CDKs. Integrins enhance growth factor-induced activation of cyclin D via extracellular signal regulated protein kinases (ERK) signaling and can activate c-Jun N-terminal kinase or activated protein 1 (AP1) related growth promoting pathways via activation of associated-associated focal adhesion kinase (FAK; Maeshima et al., Science 295:140-143, 2002; and Schuppan et al., Hepatology 38:289-291, 2003). Extracellular nucleotides have comparable effects ab initio. These may be mediated directly by P2Y receptors or indirectly by integrin inside-out signaling and consequent secondary integrin-mediated responses. With several other pathways that induce ERK activation, such as those involving protein kinase C, raf, ras, and rel, integrin signaling can be synergistic with different growth-factors resulting in enhanced cell growth.

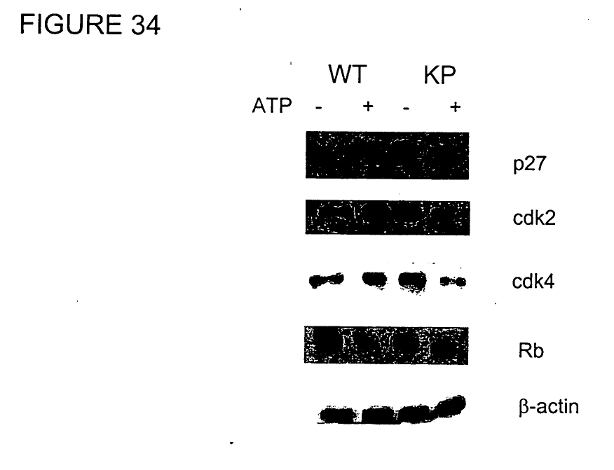

Anchorage (i.e., integrin dependent) activation of cyclic D leads to down-regulation, which is a direct consequence of cellular attachment to the extracellular matrix (ECM). Therefore, cells in suspension or where there may be integrin dysfunction display higher levels of p21 or p27, whereas integrin binding to ECM activates proteasomal degradation of these CDK inhibitors. In turn, activated FAK can induce Skp2 to promote degradation of p27, potentiating cell cycle progression via the ERK pathway. Note that cd39 null cells basally express high levels of p27 and downregulate CDK2, CDK4, and Rb following stimulation with nucleotides (FIG. 34). The inference is that in cd39 null cells there is integrin dysfunction with the potential for growth arrest. In keeping with the failure of the cd39 null cells to adhere to extracellular matrix, we also observe dysregulation of integrin ligand receptive affinity states (WOW-1 epitope), abnormal proliferative responses in vitro, and perturbation of angiogenesis in vivo.

All publications and patent applications mentioned in this specification are herein incorporated by reference to the same extent as if each independent publication or patent application was specifically and individually indicated to be incorporated by reference.

1. A method of identifying a compound capable of modulating angiogenesis in a subject, said method comprising:
a) exposing a cell expressing CD39 to a compound; and
b) assaying CD39 biological activity, wherein a decrease in CD39 biological activity in said cell, relative to CD39 biological activity in a cell not exposed to said compound, indicates that said compound is capable of decreasing angiogenesis, and an increase in CD39 biological activity in said cell, relative to CD39 biological activity in a cell not exposed to said compound, indicates that said compound is capable of increasing angiogenesis.

2. The method of claim 1, wherein said CD39 biological activity is the phosphohydrolysis of nucleoside diphosphate or triphosphate.

3. The method of claim 2, wherein said nucleoside diphosphate is selected from adenosine diphosphate (ADP) or uridine diphosphate (UDP), or said nucleoside triphosphate is selected from adenosine triphosphate (ATP) or uridine triphosphate (UTP).

4. The method of claim 1, wherein said cell is selected from the group consisting of a monocyte, a macrophage, an endothelial cell, and a cancer cell.

5. A method of identifying a compound capable of modulating CD39-associated angiogenesis in a subject, said method comprising:
a) injecting MATRIGEL® comprising one or more growth factors and a compound into a CD39 null mouse;
b) assaying for the ingrowth of blood vessels into said MATRIGEL®, wherein an increase in said ingrowth of blood vessels into said MATRIGEL®, relative to the ingrowth of blood vessels into MATRIGEL® lacking the compound, indicates that said compound is capable of promoting angiogenesis, and a decrease in said ingrowth of blood vessels into said MATRIGEL®, relative to the ingrowth of blood vessels into MATRIGEL® lacking the compound, indicates that said compound is capable of inhibiting angiogenesis.

6. The method of claim 5, wherein said one or more growth factors are selected from vascular endothelial growth factor (VEGF), sphingosine-1-phosphate, and fibroblast growth factor (FGF).

7. The method of claim 5, wherein said assaying for the ingrowth of blood vessels is performed after incubating said MATRIGEL® in said mouse for between 7 and 21 days.

8. A method of decreasing angiogenesis in a subject in need thereof, comprising administering a compound that decreases CD39 biological activity in an amount sufficient to decrease angiogenesis.

9. The method of claim 8, wherein said CD39 biological activity is the phosphohydrolysis of nucleoside diphosphate or triphosphate.

10. The method of claim 8, wherein said compound is selected from the group comprising a nucleoside analog, a peptide, an antibody, and a CD39 antisense RNA.

11. The method of claim 8, wherein said compound is antisense CD39 RNA capable of decreasing CD39 biological activity in a cell expressing CD39.

12. The method of claim 8, wherein said subject is a human.

13. The method of claim 8, wherein said subject has one or more of the following: cancer, rheumatoid arthritis, diabetic retinopathy, inflammatory bowel disease, or chronic radiation-induced proctitis.

14. A method of promoting angiogenesis in a subject in need thereof, comprising administering a compound that increases CD39 biological activity in an amount sufficient to promote angiogenesis.

15. The method of claim 14, wherein said CD39 biological activity is the phosphohydrolysis of nucleoside diphosphate or triphosphate.

16. The method of claim 14, wherein said compound is selected from a CD39 transgene in an expressible genetic construct or a peptide mimetic of CD39.

17. The method of claim 16, wherein said CD39 transgene is administered using a viral vector.

18. The method of claim 17, wherein said viral vector is an adenoviral vector.

19. The method of claim 14, wherein said subject is a human.

20. The method of claim 14, wherein said subject has one or more of the following: peripheral vascular disease, cardiovascular disease, tissue organ engraftment rejection, or sequelae of ischemic reperfusion injury.

21. The method of claim 20, wherein said peripheral vascular disease is atherosclerosis, thromboembolic disease, or Buerger's disease (thromboangiitis obliterans).

22. The method of claim 20, wherein said cardiovascular disease is myocardial infarction, heart disease, or coronary artery disease.

23. A pharmaceutical composition comprising CD39 antisense RNA in a pharmaceutically acceptable carrier, wherein said antisense RNA is capable of reducing angiogenesis in a subject.

24. A method of diagnosing an increased risk of an angiogenesis-associated condition comprising detecting the level of CD39 biological activity in a subject, wherein an increased or decreased level of CD39 biological activity indicates said subject has an increased risk of an angiogenesis-associated condition.

25. The method of claim 24, wherein said angiogenesis-associated condition is cancer or metastasis of cancer, inflammation, inflammatory bowel disease, or chronic radiation-induced proctitis, and wherein detection of an increase in said level of CD39 biological activity indicates said subject has an increased risk of cancer or metastasis of cancer, inflammation, inflammatory bowel disease, or chronic radiation-induced proctitis.

26. The method of claim 24, wherein said angiogenesis-associated condition is peripheral vascular disease, cardiovascular disease, tissue organ engraftment rejection, or sequelae of ischemic reperfusion injury, and wherein detection of a decrease in said level of CD39 biological activity indicates said subject has an increased risk of peripheral vascular disease, cardiovascular disease, tissue organ engraftment rejection, or sequelae of ischemic reperfusion injury.

27. The method of claim 26, wherein said peripheral vascular disease is atherosclerosis, thromboembolic disease, or Buerger's disease (thromboangiitis obliterans) and said cardiovascular disease is myocardial infarction, heart disease, or coronary artery disease.

28. The method of claim 24, wherein the level of CD39 biological activity is detected by assaying the level of CD39 mRNA, CD39 protein, or the phosphohydrolytic activity of CD39.

29. The method of claim 28, wherein said level of CD39 mRNA, CD39 protein, or the phosphohydrolytic activity of CD39 is detected using a biopsy.

30. A method for determining the prognosis for treatment of an angiogenesis-associated condition in a subject, said method comprising determining the level of CD39 biological activity in a sample from said subject, wherein an increase or decrease in said CD39 biological activity in said sample, relative to the amount of CD39 biological activity in a control sample, determines the prognosis for treatment of an angiogenesis-associated condition in said subject.

31. The method of claim 30, wherein said angiogenesis-associated condition is cancer or metastasis of cancer, rheumatoid arthritis, diabetic retinopathy, inflammatory bowel disease, or chronic radiation-induced proctitis, and wherein an increase in said CD39 biological activity indicates a negative prognosis and a decrease in said CD39 biological activity indicates a positive prognosis.

32. The method of claim 30, wherein said angiogenesis-associated condition is peripheral vascular disease, cardiovascular disease, tissue organ engraftment rejection, or sequelae of ischemic reperfusion injury, and wherein an increase in said CD39 biological activity indicates a positive prognosis and a decrease in said CD39 biological activity indicates a negative prognosis.

33. The method of claim 32, wherein said peripheral vascular disease is atherosclerosis, thromboembolic disease, or Buerger's disease (thromboangiitis obliterans) and wherein said cardiovascular disease is myocardial infarction, heart disease, or coronary artery disease.

34. The method of claim 30, wherein said subject is a human.

35. The method of claim 30, wherein said sample is a biopsy.
